(19) United States
(12) Patent Application Publication
(10) Pub. No.: US 2013/0078718 A1
(43) Pub. Date: Mar. 28, 2013

(54) CONVERSION OF VASCULAR ENDOTHELIAL CELLS INTO MULTIPOTENT STEM-LIKE CELLS

(71) Applicants: Damian Medici, Boston, MA (US); Bjorn Olsen, Milton, MA (US)

(72) Inventors: Damian Medici, Boston, MA (US); Bjorn Olsen, Milton, MA (US)

(73) Assignee: PRESIDENT AND FELLOWS OF HARVARD COLLEGE, Cambridge, MA (US)

(21) Appl. No.: 13/583,294

(22) PCT Filed: Mar. 9, 2011
(86) PCT No.: PCT/US11/27679

§ 371 Date: Oct. 25, 2012

Related U.S. Application Data
(60) Provisional application No. 61/312,076, filed on Mar. 9, 2010.

(51) Int. Cl. C12N 5/071

(52) U.S. Cl. CPC C12N 5/0696
USPC 435/350; 435/377; 435/363; 435/351; 435/352; 435/366

(57) ABSTRACT

Disclosed herein is a method of producing multipotent cells, comprising, activating ALK2 of isolated endothelial cells in a serum starved environment, to thereby produce isolated multipotent cells. Activation can be following a threshold period of serum starvation. Activating ALK2 is by contacting the isolated endothelial cells with TGFβ-2 and/or BMP4. The isolated endothelial cells may be human, such as primary vascular, primary microvascular endothelial cells, primary human umbilical vein endothelial cells (HUVEC) or primary human cutaneous microvascular endothelial cells (HCMEC). The activation of ALK2 significantly decreases expression of VE-cadherein of the cells and/or significantly increases expression of one or more of STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, and CD124. The multipotent cells may further be used to generate other cell types such as osteoblast-like cells, chondrocyte-like cells, adipocyte-like cells, neural-like cells, and myocyte-like cells, by incubating the isolated multipotent cells in the appropriate culture conditions for a period sufficient to induce differentiation. The induced cells express TIE-2.

### RELATED APPLICATIONS

This application claims the benefit under 35 U.S.C. §119(e) to U.S. Provisional patent application Ser. No. 61/312,076, filed Mar. 9, 2010, the contents of which are herein incorporated by reference in their entirety.

### GOVERNMENTAL SUPPORT

This invention was made with Government support under P01 AR48564 awarded by the National Institutes of Health. The Government has certain rights in the invention.

### FIELD OF THE INVENTION

The present invention relates to the field of cell biology.

### BACKGROUND OF THE INVENTION

Epithelial and endothelial cell plasticity are critical for both embryonic development and disease progression¹. Transformation of epithelial cells into mesenchymal cells (epithelial-mesenchymal transition or EMT) is a mechanism that regulates gastrulation, neural crest and somite dissociation, craniofacial development, wound healing, organ fibrosis, and tumor metastasis²⁻⁵. Similarly, endothelial-mesenchymal transition (EndMT) is a critical aspect of endocardial cushion formation during cardiac development⁶⁻¹¹ and recent studies have shown that EndMT plays an essential role in the tumor microenvironment by generating carcinoma-associated fibroblasts¹² and may be an essential mediator of cancer progression¹. Similarly, many fibroblasts formed during cardiac¹³ and renal¹⁴⁻¹⁶ fibrosis have been shown to be of endothelial origin. EndMT has also been implicated in atherosclerosis¹⁷, pulmonary hypertension¹⁸, and wound healing¹⁹. However, whether mature endothelial cells can be induced to differentiate into a variety of cell fates is unknown.

Fibrodysplasia Ossificans Progressiva (FOP) is a disabling disease in which acute inflammation causes heterotopic cartilage and bone to form in soft tissues²⁰. All FOP patients carry a heterozygous mutation (R206H) in the gene encoding the Transforming Growth Factor-beta (TGF-β)/Bone Morphogenetic Protein (BMP) type 1 receptor Activin-like kinase 2 (ALK2)²¹⁻²³. Heterotopic ossification in FOP lesions begins with fibroblasts condensation, followed by chondrogenesis. Cartilage then follows standard developmental progression to bone through endochondral ossification²⁰. The source of the heterotopic cartilage and bone formed in FOP lesions is currently unknown.

### SUMMARY OF THE INVENTION

The present invention relates to a method of producing multipotent cells from endothelial cells. The method comprises activating ALK2 in the endothelial cells, in a serum starved environment, to thereby produce multipotent cells. In one embodiment, the method further comprises subjecting the endothelial cells to a threshold period of serum starvation prior to activating ALK2. In one embodiment, activating ALK2 is by contacting the endothelial cells with TGFβ-2, and/or BMP4, and/or an analog, derivative or functional fragment thereof. In one embodiment, the TGFβ-2, BMP4, and/or analog, derivative or functional fragment thereof is contacted to the endothelial cells at a concentration of about 10 ng/ml. In one embodiment, activating ALK2 is for at least about 48 hours. In one embodiment, the threshold period of serum starvation is for at least about 24 hours. In one embodiment, the endothelial cells are selected from the group consisting of primate, equine, bovine, porcine, canine, feline and rodent. In one embodiment, the endothelial cells are human. In one embodiment, the endothelial cells are primary vascular or primary microvascular endothelial cells. In one embodiment, the endothelial cells are isolated. In one embodiment, the endothelial cells are primary human umbilical vein endothelial cells (HUVEC) or primary human cutaneous microvascular endothelial cells (HCMEC). In one embodiment, activation of ALK2 significantly decreases expression of VE-cadherein of the cells and/or to significantly increase expression of one or more of STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, CD124.

Another aspect of the present invention relates to a method of producing osteoblast-like cells, comprising incubating multipotent cells produced by the methods described herein, in osteogenic culture medium for a period sufficient to induce differentiation.

Another aspect of the present invention relates to a method of producing isolated chondrocyte-like cells, comprising incubating multipotent cells produced by the methods described herein, in chondrogenic culture medium for a period sufficient to induce differentiation.

Another aspect of the present invention relates to a method of producing adipocyte-like cells, comprising incubating multipotent cells produced by the methods described herein, in adipogenic culture medium for a period sufficient to induce differentiation.

Another aspect of the present invention relates to method of producing neural-like cells, comprising incubating multipotent cells produced by the methods described herein, in neuralgenic culture medium for a period sufficient to induce differentiation.

Another aspect of the present invention relates to a method of producing myocyte-like cells, comprising incubating multipotent cells produced by the methods described herein, in myogenic culture medium for a period sufficient to induce differentiation.

Another aspect of the present invention relates to a method of producing cardiomyocyte-like cells, comprising incubating multipotent cells produced by the methods described herein, in cardiomyogenic culture medium for a period sufficient to induce differentiation.

Another aspect of the present invention relates to an isolated multipotent human mesenchymal cell, or population thereof, wherein the multipotent human mesenchymal cell expresses transcripts for STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, or CD124, or combinations thereof, and has a normal karyotype.

Another aspect of the present invention relates to an isolated multipotent human mesenchymal cell or population thereof that expresses transcripts for TIE-2 and FSP-1. In one embodiment, the isolated multipotent human mesenchymal cell is produced by a method described herein.

Another aspect of the present invention relates to an isolated multipotent cell or population thereof, produced by the methods described herein, that expresses TIE-2 and FSP-1.

In one embodiment, the isolated multipotent cell or population thereof, has fibroblast-like morphology. In one embodiment, the isolated multipotent cell or population thereof, is human.

Another aspect of the present invention relates to an isolated cell or population thereof, that expresses one or more osteoblast specific markers and TIE-2. In one embodiment, the osteoblast specific marker is osteocalcin or osterix. In one embodiment, the isolated cell or population thereof, is produced by a method described herein.

Another aspect of the present invention relates to an isolated cell or population thereof, that expresses one or more chondrocyte specific markers and TIE-2. In one embodiment, the chondrocyte specific marker is SOX9. In one embodiment, the isolated cell or population thereof, is produced by a method described herein.

Another aspect of the present invention relates to an isolated cell or population thereof, that expresses one or more adipocyte specific markers and TIE-2. In one embodiment, the adipocyte specific marker is PPARβ2. In one embodiment, the isolated cell or population thereof, is produced by a method described herein.

Another aspect of the present invention relates to an isolated cell or population thereof, that expresses one or more neuronal specific markers and TIE-2. In one embodiment, the neuronal specific marker is neurofilament-L, neuron-specific enolase, neurofilament 200 and/or neuron-specific beta III-tubulin. In one embodiment, the isolated cell or population thereof, is produced by a method described herein.

Another aspect of the present invention relates to an isolated cell or population thereof, that expresses one or more myocyte specific markers and TIE-2. In one embodiment, the myocyte specific marker is myogenin, MyoD, and/or slow muscle myosin. In one embodiment, the isolated cell or population thereof, is produced by a method described herein.

Another aspect of the present invention relates to an isolated cell or population thereof, that expresses one or more cardiomyocyte specific markers and TIE-2. In one embodiment, the cardiomyocyte specific marker is cardiac troponin-1. In one embodiment, the isolated cell or population thereof, is produced by a method described herein.

Another aspect of the present invention relates to a tissue generated from the cell or population thereof, described herein. In one embodiment, the tissue is selected from the group consisting of skeletal muscle, bone, cartilage, heart, connective tissue, adipose tissue, and neural tissue.

### DEFINITIONS

As used herein, the term “primary cell”, refers to a cell that is obtained directly from an organism. The cells can undergo several rounds of proliferation, or rounds of passages, to expand the population prior to use in the methods described herein to produce the stem-like cells and cells derived therefrom, described herein. In one embodiment, the primary cells undergo few to no rounds of proliferation prior to use.

The term “multipotent” is used to refer to cells that can differentiate into a number of different cell types, especially those of a closely related family of cells. Such cells are also referred to in the art as “stem cells” or “stem-like cells”, as the term is used herein.

The term “purified” is used to refer to a molecule that is substantially free of other cellular material, culture medium, chemical precursors or other chemicals. For example, purified is about 80% free, about 85% free, about 90% free, or about 95% free from other materials.

By “isolated” is meant a material that is free to varying degrees from components which normally accompany it as found in its native state. “Isolate” denotes a degree of separation from original source or surroundings. For example, an isolated cell can be removed from an animal and placed in a culture dish or another animal. Isolated is not meant as being removed from all other cells.

By “population” is meant at least 2 cells. In a preferred embodiment, population is at least 5, 10, 50, 100, 500, 1000, or more cells. The invention relates to cells obtained by the methods described herein. As such, use of the term “cell” or “isolated cell” when describing the invention is also intended to describe a population of such cells. Populations of cells and isolated cells described herein are also encompassed by the present invention. A population may be comprised of genetically identical cells. The population may contain only a single cell type, or may contain multiple cell types. For example, the population may contain a single cell type described herein (e.g., mesenchymal stem-like cells, chondrocyte-like cells, osteoblast-like cells, adipocyte-like cells, neural-like cells, myocyte-like cells, cardiomyocyte-like cells) or may contain a plurality of different cell types, (e.g., at various stages of differentiation). The population may contain unrelated cell types as well.

The term “functional fragment” as used herein in connection with an agent, refers to a portion of the agent molecule that retains a significant amount of activity of a desired function of the whole, intact functional molecule (e.g., the ability of TGFβ2, BMP4, BMP2 or BMP7 to activate ALK2). Functional fragments can also be generated from derivatives or variants.

The term “cell type-specific marker” refers to any molecular moiety (e.g., protein, peptide, mRNA or other RNA species, DNA, lipid, carbohydrate) whose presence in a cell indicates cell type. Typically the cell type-specific marker is either uniquely present on or in the cell type, or present at a higher level on or in a particular cell type or cell types of interest, than on or in many other cell types. In some instances a cell type specific marker is present at detectable levels only on or in a particular cell type of interest. However, it will be appreciated that useful markers need not be absolutely specific for the cell type of interest. In general, a cell type specific marker for a particular cell type is present at levels at least 3 fold greater in that cell type than in a reference population of cells. More preferably the cell type-specific marker is present at levels at least 4-5 fold, between 5-10 fold, or more than 10-fold greater than its average level of presence (e.g., expression) in a reference population. In some instances, the presence of one or more given cell type-specific markers, in the absence or otherwise reduced expression of another marker, is used to identify a particular cell type. Preferably detection or measurement of a cell type-specific marker makes it possible to distinguish the cell type or types of interest from cells of many, most, or all other types. In general, the presence and/or abundance of most markers may be determined using standard techniques such as Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein microarray analysis, mass spectrometry, etc. In some instances, the presence of a specific combination of different moleculare moiteis on or in a cell can be used as the cell type-specific marker. The marker can be present on the surface of the cell (e.g., an antigenic marker) or otherwise present within the cell. Cell type-specific markers for the various cell types are known in the art and routinely detected by conventional means.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. More than one dose may be required for treatment of a disease or condition.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition. More than one dose may be required for prevention of a disease or condition.

The term “subject” includes organisms which are capable of suffering from a disease, disorder or injury, who could otherwise benefit from the administration of a compound or composition of the invention, such as human and non-human animals. The terms subject and individual are used interchangeably herein. The term “non-human animals” of the invention includes all vertebrates, including, without limitation, mammals (e.g., rodent (mice), primate, canine, equine, bovine, feline, porcine) and non-mammals, such as non-human primates. Specific subjects include, without limitation, humans, sheep, dog, cow, horses, chickens, mice, rats, hamster, amphibians, reptiles, amphibians, etc. Cells described herein can be derived from any such subject described herein.

The term “therapeutically effective amount” refers to an amount that is sufficient to effect a therapeutically or prophylactically significant reduction in a symptom associated with a disease, disorder or injury being treated, when administered to a typical subject with that condition. A therapeutically significant reduction in a symptom is, e.g. about 10%, about 20%, about 30%, about 40%, about 50%, about 60%, about 70%, about 80%, about 90%, about 100%, about 125%, about 150% or more as compared to a control or non-treated subject.

The compositions as disclosed herein can be administered in prophylactically or therapeutically effective amounts. A prophylactically effective amount means that amount necessary, at least partly, to attain the desired effect, or to delay the onset of, inhibit the progression of, or halt altogether, the onset or progression of the particular disease or disorder being treated.

Such amounts for therapy or prophylaxis will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a lower dose or tolerable dose can be administered for medical reasons, psychological reasons or for virtually any other reasons.

The term “nervous system disease” or “disease of the nervous system” refers to any condition characterized by the progressive loss of neurons, due to cell death, in the central or peripheral nervous system of a subject.

The term “pharmaceutical composition” refers to compositions or formulations that usually comprise an excipient, such as a pharmaceutically acceptable carrier that is conventional in the art and that is suitable for administration to a subject, such as a mammals, and preferably humans. Such compositions can be specifically formulated for administration via one or more of a number of routes described herein, including but not limited to, oral, ocular and nasal administration and the like. The pharmaceutical composition may further provide a suitable environment for preservation of the viability of any cells contained therein to be administered in the composition.

The “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human.

The term “administration” as used herein refers to the presentation of compositions described herein to humans and animals in effective amounts, and includes all routes for dosing or administering drugs. Such routes, include, without limitation, parenteral, systemic, enteral, and topical. The phrases “parenteral administration” and “administered parenterally” as used herein means modes of administration other than enteral and topical administration, usually by injection, and includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The term “analog” as used herein in connection with an agent, refers to an agent that retains the same biological function (i.e., binding to a receptor, activation of ALK2) and/or structure as the molecule (e.g., polypeptide or nucleic acid) it is an analogue of. Examples of analogs include peptidomimetics, peptide nucleic acids, small and large organic or inorganic compounds. Analogs can also be made from derivatives and variants of a molecule described herein.

The term “derivative” or “variant” as used herein refers to a peptide, chemical or nucleic acid that differs from the naturally occurring polypeptide or nucleic acid by one or more amino acid or nucleic acid deletions, additions, substitutions or side-chain modifications. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative.

As used herein, the twenty conventional amino acids and their abbreviations follow conventional usage. See IMMUNOLOGY—A SYNTHESIS, 2nd Edition, (E. S. Golub and D. R. Gren, Eds.), Sinauer Associates: Sunderland, Mass., 1991, incorporate herein by reference for any purpose. Stereoisomers (e.g., d-amino acids) of the twenty conventional amino acids; unnatural amino acids such as α-,α.-disubstituted amino acids, N-alkyl amino acids, lactic acid, and other unconventional amino acids may also be suitable components for polypeptides of the invention. Examples of unconventional amino acids include: 4-hydroxyproline, gamma-carboxyglutamate, epsilon-N,N,N-trimethyllysine, epsilon-N-acetyllysine, O-phosphoserine, N-acetylserine, N-formylmethionine, 3-methylhistidine, 5-hydroxylysine, sigma-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:

1) hydrophobic; norleucine (Nor), Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

Conservative amino acid substitutions may involve exchange of a member of one of these classes with another member of the same class. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions may involve the exchange of a member of one of these classes for a member from another class. Such substituted residues may be introduced into regions of a human protein that are homologous with non-human proteins, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within .+−0.2 is included. In certain embodiments, those that are within .+−0.1 are included, and in certain embodiments, those within +0.5 are included.

It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within .+−0.2 is included, in certain embodiments, those that are within .+−0.1 are included, and in certain embodiments, those within .+−.0.5 are included. One may also identify epitopes from primary amino acid sequences on the basis of hydrophilicity. These regions are also referred to as “epitopic core regions.”

A skilled artisan will be able to determine suitable variants of the polypeptide (e.g., TGFβ-2 or BMP4) as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three dimensional structure. In certain embodiments, one skilled in the art may choose to not make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult, 1996, Curr. Op. in Biotech. 7:422-427; Chou et al., 1974, Biochemistry 13:222-245; Chou et al., 1974, Biochemistry 113:211-222; Chou et al., 1978, Adv. Enzymol. Relat. Areas Mol. Biol. 47:45-148; Chou et al., 1979, Ann. Rev. Biochem. 47:251-276; and Chou et al., 1979, Biophys. J. 26:367-384. Moreover, computer programs are currently available to assist with predicting secondary structure. One method of predicting secondary structure is based upon homology modeling. For example, two polypeptides or proteins that have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., 1999, Nucl. Acid. Res. 27:244-247. It has been suggested (Brenner et al., 1997, Curr. Op. Struct. Biol. 7:369-376) that there are a limited number of folds in a given polypeptide or protein and that once a critical number of structures have been resolved, structural prediction will become dramatically more accurate.

Additional methods of predicting secondary structure include “threading” (Jones, 1997, Curr. Opin. Struct. Biol. 7:377-87; Sippl et al., 1996, Structure 4:15-19), “profile analysis” (Bowie et al., 1991, Science 253:164-170; Gribskov et al., 1990, Meth. Enzym. 183:146-159; Gribskov et al., 1987, Proc. Nat. Acad. Sci. 84:4355-4358), and “evolutionary linkage” (See Holm, 1999, supra; and Brenner, 1997, supra).

### BRIEF DESCRIPTION OF THE DRAWINGS

FIG. 1A-FIG. 1F shows data that indicates that TGF-β2 and BMP4 induce endothelial-mesenchymal transition via ALK2 signaling. FIG. 1A) immunoblotting confirming EndMT with decreased expression of VE-cadherin and increased expression of FSP-1 and α-SMA in cells treated with TGF-β2 or BMP4. TIE2 levels remained constant. FIG. 1B) Real-time quantitative PCR showing TGF-β2- or BMP4-induced increases in expression of genes associated with EndMT. Data represent mean (n=3)±s.d.; P<0.05 (ANOVA) for all TGF-β2 or BMP4 treated cells compared to control cells. FIG. 1C) Immunoprecipitation confirming phosphorylation of ALK2 by TGF-β2 or BMP4. FIG. 1D) Immunoblotting showing increased phosphorylation of Smad5 by TGF-β2 or BMP4. FIG. 1E) Immunoblotting confirming knockdown of ALK2 expression by ALK2 siRNA. FIG. 1F) Immunoblotting showing increased expression of FSP-1 in TGF-β2 or BMP4 treated endothelial cells transfected with negative control siRNA. This increased expression was inhibited in cells transfected with ALK2 siRNA. β-actin was used as an internal control for all immunoblotting experiments.

FIG. 2A-FIG. 2B shows data that indicates endothelial cells transformed by treatment with TGF-β2 or BMP4 express mesenchymal stem cell markers. FIG. 2A) Immunoblotting confirming increased protein expression of mesenchymal stem cell markers STRO-1, CD44, and CD90 in cells treated with TGF-β2 or BMP4. Human bone marrow derived mesenchymal stem cells (MSC) express these markers, whereas human corneal fibroblasts (HCF) do not. β-actin was used as an internal control. FIG. 2B) Real-time quantitative PCR analysis showing increased gene expression of various stem cell markers in cells exposed to TGF-β2 or BMP4. Data represent mean (n=3)±s.d.; P<0.01 (ANOVA) for all TGF-β2 or BMP4 treated cells compared to control cells.

FIG. 3A-FIG. 3G shows data that indicates endothelial derived mesenchymal stem cells exhibit multipotency. FIG. 3A-FIG. 3C) Real-time quantitative PCR analysis showing increased expression of osteoblast (osteocalcin, osterix), chondrocyte (COL2A1, SOX9), or adipocyte (adiponectin, PPARγ2) markers in cells treated with TGF-β2 or BMP4 for 48 hours followed by exposure to osteogenic (A), chondrogenic (B), or adipogenic (C) culture medium. Data represent mean (n=3)±s.d.; P<0.01 (ANOVA) for all TGF-β2 or BMP4 treated cells compared to control cells. FIG. 3D) Immunoblotting showing increased expression of STRO-1 in TGF-β2- or BMP4-treated endothelial cells transfected with negative control siRNA. Increased expression of STRO-1 did not occur in cells transfected with ALK2 siRNA. β-actin was used as a loading control. FIG. 3E-FIG. 3G) Real-time quantitative PCR analysis showing increased expression of osteoblast (osteocalcin, osterix), chondrocyte (COL2A1, SOX9), or adipocyte (adiponectin, PPARγ2) markers in HCMECs transfected with control siRNA and treated with TGF-β2 or BMP4 for 48 hours, followed by exposure to osteogenic (E), chondrogenic, (F) or adipogenic (G) culture medium. Treatment with ALK2 siRNA prevented these increases. Data represent mean (n=3)±s.d.; P<0.01 (ANOVA) for all cells treated with TGF-β2 or BMP4 and control siRNA compared to cells treated with vehicle and control siRNA.

FIG. 4A-FIG. 4E shows data that indicates constitutively active ALK2 promotes endothelial-mesenchymal transition. FIG. 4A) Immunoblotting showing positive expression of the His-Tag on wild-type (WT) and mutant (Mut) ALK2 in infected endothelial cells, as well as increased expression of total ALK2. β-actin was used as an internal control. FIG. 4B) Immunoprecipitation demonstrating constitutive tyrosine phosphorylation (P-Y) of the mutant ALK2 receptor. FIG. 4C) Immunoblotting showing constitutive Smad5 phosphorylation in endothelial cells expressing mutant ALK2. β-actin was used as a loading control. FIG. 4D) Immunoblotting showing deceased expression of the endothelial marker VE-cadherin and increased expression of the mesenchymal markers FSP-1 and α-SMA. TIE2 levels remained constant. β-actin was used as an internal control. FIG. 4E) Real-time quantitative PCR analysis showing increased expression of genes known to be associated with EndMT. Data represent mean (n=3)±s.d.; P<0.05 (ANOVA) for all Mut treated cells compared to WT cells.

FIG. 5A-FIG. 5E shows data that indicates the formation of endothelial derived multipotent stem cells by constitutively active ALK2. FIG. 5A) Immunoblotting showing expression of the mesenchymal stem cell markers STRO-1, CD44, and CD90 in endothelial cells expressing mutant ALK2. β-actin was used as an internal control. FIG. 5B) Real-time quantitative PCR analysis showing increased expression of various mesenchymal stem cell markers in endothelial cells containing the mutant ALK2 (Mut) construct compared to vector or wild-type ALK2 (WT) infected cells. Data represent mean (n=3)±s.d.; P<0.01 (ANOVA) for all Mut treated cells compared to WT treated cells. FIG. 5C-FIG. 5E) Real-time quantitative PCR analysis showing increased expression of osteoblast (osteocalcin, osterix), chondrocyte (COL2A1, SOX9), or adipocyte (adiponectin, PPARγ2) markers in cells treated with mutant ALK2 for 48 hours followed by exposure to osteogenic (C), chondrogenic (D), or adipogenic (E) culture medium. Data represent mean (n=3)±s.d.; P<0.01 (ANOVA) for all Mut treated cells compared to WT cells.

FIG. 6 shows data that indicates endothelial cell differentiation in Fibrodysplasia Ossificans Progressiva. Immunoblotting showing that bone marrow-derived mesenchymal stem cells (MSC) do not express the endothelial marker TIE2, nor do osteoblasts, chondrocytes, or adipocytes (as identified by expression of their characteristic molecular markers osteocalcin, SOX9, and PPARγ2) derived from MSCs grown in appropriate differentiation medium, or primary human osteoblasts, chondrocytes, or adipocytes. Osteoblasts, chondrocytes, and adipocytes derived from endothelial cells stimulated by TGF-β2 or BMP4, followed by exposure to appropriate differentiation medium, do express TIE2.

FIG. 7A-FIG. 7C shows data that indicates the differentiation potential of human bone marrow derived mesenchymal stem cells (MSC) and human corneal fibroblasts (HCF).

Real-time quantitative PCR analysis showing expression of osteoblast (osterix), chondrocyte (SOX9), or adipocyte (PPARγ2) markers in cells exposed to osteogenic (A), chondrogenic (B), or adipogenic (C) culture medium. Data represent mean (n=3)±s.d.; *P<0.01 (student's t test) compared to cells in growth medium.

FIG. 8 shows data that indicates mutant ALK2 induces expression of mesenchymal stem cell markers in endothelial cells. Immunoblotting of endothelial cell fractions (from fluorescence activated cell sorting separated mutant ALK2 (Mut) treated endothelial cells that express the His-Tag (+) and those that do not (−)) showing that only cells containing the His-Tag express the mesenchymal marker FSP-1 and the stem cell markers STRO-1, CD44, and CD90. Bone marrow derived mesenchymal stem cells (MSC) express all of these markers, while human corneal fibroblasts (HCF) only express FSP-1. Neither MSC nor HCF express the endothelial marker TIE2. β-actin was used as an internal control.

FIG. 9A-FIG. 9B shows data that indicates the quantification of Tie2-positive osteoblasts and chondrocytes in heterotopic bone and cartilage. Graphic analysis of the percentage Tie2-positive cells in (A) wild-type bone and cartilage from the mouse knee joint (n=3) vs. heterotopic bone and cartilage from mutant ALK2 transgenic mice (n=7) and (B) normal human bone and cartilage from the hip joint (n=3) vs. heterotopic bone and cartilage from FOP patients (n=3). Data represent mean±s.d.; *P<0.01 (student's t test) compared to WT or normal tissue.

FIG. 10A-FIG. 10C shows data that indicates osteoblasts and chondrocytes from heterotopic bone and cartilage express the endothelial marker vWF. (A) Immunoblotting showing that levels of vWF are decreased, but still strongly expressed in HCMECs infected with mutant ALK2 (Mut) expression construct compared to cells infected with wild-type ALK2 (WT) or vector. (B) Quantification of vWF-positive cells in wild-type (WT) cartilage and bone (n=3) vs. heterotopic cartilage and bone (n=7). Data represent mean±s.d.; *P<0.01 (student's t test) compared to WT. (C) Quantification of vWF-positive cells in normal cartilage and bone (n=3) vs. heterotopic cartilage and bone (n=3) from FOP lesions. Data represent mean±s.d.; *P<0.01 (student's t test) compared to normal tissue.

FIG. 11A-FIG. 11C show results from experiments that indicate constitutively active ALK2 promotes endothelial-mesenchymal transition. FIG. 11A is a photograph of an immunoblot that shows positive expression of the His-Tag on wild-type (WT) and mutant (Mut) ALK2 in infected endothelial cells, as well as increased expression of total ALK2. β-actin was used as an internal control. FIG. 11B is a photograph of results from an immunoprecipitation experiment. The immunoprecipitation demonstrated constitutive tyrosine phosphorylation (P-Y) of the mutant ALK2 receptor. FIG. 11C is a photograph of an immunoblot that shows deceased expression of the endothelial markers VE-cadherin, CD31, and vWF and increased expression of the mesenchymal markers FSP-1, α-SMA, and N-cadherin in endothelial cells expressing mutant ALK2 but not in cells infected with vector or wild-type ALK2 adenoviral constructs. TIE2 levels remained constant. β-actin was used as an internal control.

FIG. 12A-FIG. 12B show results from experiments that indicate formation of endothelial derived multipotent stem-like cells induced by constitutively active ALK2. FIG. 12A is immunoblotting showing expression of the mesenchymal stem cell markers STRO-1, CD10, CD44, CD71, CD90, and CD117 in endothelial cells expressing mutant ALK2. Human bone marrow derived mesenchymal stem cells (MSC) express these markers, but human corneal fibroblasts (HCF) do not. β-actin was used as an internal control. FIG. 12B is immunoblotting showing increased expression of osteoblast (osterix), chondrocyte (SOX9), or adipocyte (PPARγ2) markers in cells treated with mutant ALK2 for 48 h followed by exposure to osteogenic, chondrogenic, or adipogenic culture medium.

FIG. 13A-FIG. 13B show results from experiments that indicate TGF-β2 and BMP4 activate ALK2 and induce endothelial-mesenchymal transition. FIG. 13A is immunoblotting of immunoprecipitates confirming phosphorylation of ALK2 by 15 min of TGF-β2 or BMP4 stimulation. FIG. 13B is immunoblotting confirming EndMT with decreased expression of VE-cadherin, CD31, and vWF and increased expression of FSP-1, α-SMA, and N-cadherin in cells treated with TGF-β2 or BMP4. TIE2 levels remained constant.

FIG. 14A-FIG. 14B show results from experiments that indicate endothelial cells transformed by treatment with TGF-β2 or BMP4 express mesenchymal stem cell markers and exhibit multipotency. FIG. 14A shows immunoblotting confirming increased protein expression of mesenchymal stem cell markers STRO-1, CD10, CD44, CD71, CD90, and CD117 in cells treated with TGF-β2 or BMP4. β-actin was used as an internal control. FIG. 14B shows immunoblotting showing increased expression of osteoblast (osterix), chondrocyte (SOX9), or adipocyte (PPARγ2) markers in cells treated with TGF-β2 or BMP4 for 48 h followed by exposure to osteogenic, chondrogenic, or adipogenic culture medium, respectively.

FIG. 15A-FIG. 15B show results from experiments that indicate ALK2 is necessary for EndMT. FIG. 15A shows immunoblotting confirming knockdown of ALK2 expression by ALK2 siRNA in HUVEC and HCMEC cultures. FIG. 15B shows immunoblotting showing increased expression of FSP-1 and STRO-1 in TGF-β2 or BMP4 treated endothelial cells transfected with negative control siRNA, but inhibition of this expression in cells transfected with ALK2 siRNA. β-actin was used as an internal control.

FIG. 16A-FIG. 16D shows results from experiments that indicate quantification of TIE2 and vWF positive osteoblasts and chondrocytes in heterotopic bone and cartilage. FIG. 16A-16D are graphic representations of the percentage TIE2 (A, C) and vWF (B, D) positive cells in normal human bone and cartilage from the hip joint (n=3) vs. heterotopic bone and cartilage from FOP patients (n=3) and wild-type (WT) bone and cartilage from the mouse knee joint (n=3) vs. heterotopic bone and cartilage from Cre-dependent mutant ALK2 transgenic mice (n=7). Data represent mean±s.d.; *P<0.01 (student's t test) compared to normal or wild-type tissue.

FIG. 17A-FIG. 17B show results from experiments that indicate expression analysis of EndMT-inducing transcription factors. FIGS. 17A and 17B are bar graphs of data from multiplex ELISA analysis showing increased expression of Snail, Slug, ZEB-1, SIP-1, LEF-1, and Twist in cells expressing mutant ALK2 compared to wild-type (WT) ALK2 or vector (A), and in cells stimulated with TGF-β2 or BMP4 compared to vehicle (B). Data represent mean (n=3)±s.d.; P<0.05 (ANOVA) for all mutant ALK2 expressing cells compared to WT ALK2 expressing cells and all TGF-β2 or BMP4 treated cells compared to vehicle treated cells.

FIG. 18 show results from experiments that indicate mutant ALK2 induces expression of mesenchymal stem cell markers in endothelial cells. FIG. 3 shows immunoblotting of endothelial cell fractions isolated by FACS showing that only cells containing the mutant ALK2 His tag express the mesenchymal marker FSP-1 and the stem cell markers STRO-1, CD44, and CD90.

FIG. 19 shows results from experiments that indicate the differentiation potential of human bone marrow derived mesenchymal stem cells (MSC) and human corneal fibroblasts (HCF). Immunoblotting showing expression of osteoblast (osterix), chondrocyte (SOX9), or adipocyte (PPARγ2) markers in MSCs, but not in HCFs, exposed to osteogenic, chondrogenic, or adipogenic culture medium. When cultured in growth medium, MSCs did not differentiate.

FIG. 20A-FIG. 20B shows results from experiments that indicate assessment of EndMT inhibitors. FIG. 20A-20B are immunoblots showing inhibition of TGF-β2-induced decreases in CD31 and increases in FSP-1 and CD44 expression by dorsomorphin (A), BMP7 or VEGF (B) in HUVECs.

FIG. 21A-FIG. 21D show results from experients that indicate ligand specificity in ALK2 signaling. FIG. 21A shows immunoblotting demonstrating phosphorylation of Smad2 (P-Smad2) and Smad5 (P-Smad5) in HUVECs treated with TGF-β2 or BMP4 for 1 h, but phosphorylation of only Smad5 in those treated with BMP7. FIG. 21B shows immunoprecipitation of ALK2 showing the presence of ALK5 in precipitates of lysates from cells treated with TGF-β2 or BMP4 for 15 min. No ALK5 was observed in precipitates of lysates from cells treated with vehicle or BMP7. FIG. 21C shows immunoblotting showing that HUVECs expressing the mutant ALK2 protein found in FOP have phosphorylation levels of both Smad2 and Smad5. FIG. 21D shows immunoprecipitation demonstrating the presence of both ALK2 and ALK5 in lysates from cells expressing mutant ALK2, but not vector or wild-type ALK2.

FIG. 22A-FIG. 22C show results from experiments that indicate ALK receptor specificity in mediating EndMT. FIG. 22A is an immunoblot showing expression knockdown of all ALK receptors (ALK1-ALK7) in HUVECs using siRNA duplexes specific for each receptor. FIG. 22B-22C are graphical representations of data from ELISA analysis showing that TGF-β2- or BMP4-dependent decreases in VE-cadherin (B) and increases in CD44 (C) are inhibited by ALK2 siRNA or ALK5 siRNA, but not siRNA specific for ALK1, ALK3, ALK4, ALK6, or ALK7. Data represent mean (n=3)±s.d.; *P<0.01 (student's t test) compared to control siRNA.

FIG. 23A-FIG. 23B show results from experiments that indicate retention of endothelial markers after EndMT. FIG. 23A is an immunoblot showing a slight decrease in expression of TIE2 in HCMECs after 96 h of exposure to TGF-β2 or BMP4. FIG. 23B is an immunoblot showing that bone marrow-derived mesenchymal stem cells (MSC) do not express the endothelial markers TIE2, vWF, VE-cadherin, and TIE1, nor do osteoblasts, chondrocytes, or adipocytes (as identified by expression of their characteristic molecular markers osteocalcin, SOX9, and PPARγ2) derived from MSCs grown in appropriate differentiation medium, or primary human osteoblasts, chondrocytes, or adipocytes. Osteoblasts, chondrocytes, and adipocytes derived from endothelial cells stimulated by TGF-β2 or BMP4, followed by exposure to appropriate differentiation medium, do express these markers.

FIG. 24 shows results from experiments that show expression analysis of endothelial markers in stem cells. Immunoblotting showing that bone marrow derived hematopoietic stem cells (HSC) express TIE2 and trace amounts of TIE1, but not other endothelial markers (vWF and VE-cadherin) expressed in endothelial derived stem-like cells (HCMEC) transformed by TGF-β2. Bone marrow derived mesenchymal stem cells (MSC) do not express any of these markers.

FIG. 25A-FIG. 25C shows results from experiments that indicate the homogeneity and clonality of HUVECs and HCMECs. Flow cytometry analysis of HUVEC and HCMEC populations demonstrated no positive staining for fibroblast (FSP-1), smooth muscle cell (α-SMA), or pericyte (NG2) markers (data not shown). FIG. 25A shows immunoblotting for VE-cadherin and CD31 using lysates from three clonal populations of HUVECs and HCMECs expanded from single endothelial cells. FIG. 25B shows results from multiplex ELISA analysis demonstrating decreased expression of VE-cadherin and increased expression of FSP-1, CD44, and CD90 in the three clonal populations of HUVEC and HCMEC treated with TGF-β2 for 48 h. Data represent mean (n=3)±s.d.; *P<0.05 (student's t test) compared to vehicle treated cells. Three clonal populations of HUVEC and HCMEC transformed by TGF-β2 stained positive for osteoblasts (alkaline phosphatase [AP] and alizarin red [AR]), chondrocytes (alcian blue [AB]), or adipocytes (oil red O [OR]), after exposure to osteogenic, chondrogenic, or adipogenic culture medium, respectively (data not shown).

FIG. 26 contains photographs of immunoblot analysis that provides biochemical evidence of endothelial cell differentiation to myocytes, cardiomyocytes, and neurons. Immunoblotting showing protein expression of myocyte marker MyoD1, cardiomyocyte marker Troponin I, or neuronal marker Neurofilament-L in HCMEC cultures treated with vehicle or recombinant TGF-β2 then exposed to myogenic, cardiomyogenic, or neurogenic culture medium, respectively. Endothelial cells transformed into mesenchymal stem-like cells by TGF-β2 showed expression of these markers after incubation in appropriate differentiation medium. β-actin was used as a loading control.

### DETAILED DESCRIPTION OF THE INVENTION

Aspects of the invention relate to the discovery that activation of ALK2 in endothelial cells, in the context of a serum starved environment, induces endothelial-mesenchymal transition (EndMT), converting the endothelial cells to multipotent mesenchymal stem-like cells. The generated mesenchymal stem-like cells possess an ability to differentiate that is similar to mesenchymal stem cells in vivo. The mesenchymal stem-like cells, and the differentiated cells produced therefrom, serve a variety of uses, such as diagnostic, stem-cell therapy, tissue engineering, and pharmaceutical intervention.

One aspect of the present invention relates to a method of producing the multipotent mesenchymal stem-like cells. The method comprises activating ALK2 of endothelial cells in a reduced serum environment, to thereby produce isolated multipotent mesenchymal stem-like cells. In one embodiment, the endothelial cells are subjected to a threshold period of serum starvation prior to activating ALK2. In one embodiment, the method is performed in vitro, with isolated endothelial cells, producing isolated multipotent mesenchymal stem-like cells. In one embodiment, the endothelial cells are actively growing (e.g., not quiescent) just prior to the serum starvation. The endothelial cells may be grown in any acceptable endothelial cell medium sufficient to promote growth.

A variety of endothelial cell growth mediums are known in the art. Alternatively, an endothelial basal medium can be supplemented appropriately (e.g., with all of the growth factors, cytokines and supplements necessary for optimal growth of endothelial cells) to promote growth of the specific endothelial cells used in the methods. Useful supplements, growth factors and cytokines include, without limitation, epidermal growth factor, basic fibroblast growth factors, insulin-like growth factor, vascular endothelial growth factor, bovine brain extract, fetal bovine serum, ascorbic acid, heparin, and hydrocortisone. In one embodiment, the medium is specific for the specific type of endothelial cell used in the method (e.g., microvascular endothelial cell growth medium for microvascular endothelial cells).

The ALK2 is activated in the endothelial cells while they are in a serum starved environment. The terms “serum starved environment” and “serum starvation” are used herein to describe the incubation conditions and state of cells grown in serum free medium or medium that contains very low serum. In one embodiment, the serum level is low enough to induce cell cycle arrest (e.g., G0-G1 arrest). In one embodiment, low serum is less than 10% serum in the media in which the cells are incubated. Lower levels of serum will also be useful in generating the appropriate response. For example, medium with <10% (e.g., <9%, <8%, <7%, <6% and <5%, can be used). Even lower serum levels may also be used, for example, <4% serum, <3%, <2%, and <1% serum can be used. Medium with only trace amounts or with no detectable amount of serum may also be used.

In one embodiment, the endothelial cells are subjected to a threshold period of serum starvation prior to activating ALK2. In one embodiment, activation of ALK2 is initiated coincidentally with initiation of serum starvation. In one embodiment, an activator of ALK2 is added to the cells by mixing with a fresh application of the serum free or low serum medium, which is then applied to the cells.

In one embodiment, the cells are incubated in Human Endothelial Serum Free Medium (GIBCO) for a period of about 24 hours prior to all treatment. In one embodiment, cytokines to stimulate EndMT are added to the cells by way of addition to freshly applied serum free or low serum (e.g., Human Endothelial Serum Free Medium). In one embodiment, the cells are then incubated another 48 hours to thereby induce EndMT. Alternatively, other methods of inducing growth arrest may also be used to substitute for serum starvation in the methods described herein. The useful threshold periods and activation periods described herein would equally apply to such methods.

The threshold period of serum starvation can be used to achieve a cellular state in which the endothelial cells within a population of cells can be induced to undergo EndMT. As such, an entire population of cells undergoing asynchronous growth, will likely contain a subpopulation of cells that will achieve this state at various times upon exposure to serum starvation conditions. Therefore, a given threshold period of serum starvation will cause a percentage of cells within a population to become responsive to EndMT. Shorter threshold periods are envisioned to possibly affect fewer cells within the population, whereas longer period are envisioned to affect a larger percentage of cells. The optimal threshold period of serum starvation is determined by the ordinary skilled artisan for a given cell type under specified culture conditions. Useful threshold periods range from at least about 1 hour, at least about 2 hours, at least about 3 hours, . . . to at least about 24 hours. In one embodiment, the threshold period of serum starvation is at least about 12 hours. In one embodiment, the threshold period is at least about 24 hours. In one embodiment, the threshold period is at least about 36 hours. In one embodiment, the cells continue to be incubated in serum free to very low serum medium upon activation of ALK2.

Endothelial cells useful in the present invention include, endothelial cells from large and small blood vessels (e.g., microvascular cells). Examples of endothelial cells from large blood vessels include, without limitation, umbilical vein endothelial cells, umbilical artery endothelial cells, pulmonary artery endothelial cells, saphenous vein endothelial cells. Other useful endothelial cells, include, without limitation, lung microvascular cells, coronary artery endothelial cells, cutaneous microvascular endothelial cells, aortic endothelial cells. The endothelial cells may be mature or immature. In one embodiment, the endothelial cells are human. Endothelial cells may be primary cells or cells grown in culture for an extended period of time.

ALK2 is a BMP type I receptor. It is a serine/threonine kinase that is phosphorylated (e.g., tyrosine phosphorylated) in response to binding by its ligand. It activates a variety of downstream signaling molecules, including SMA.

ALK2 (also called ActRIa or ActRI) is activated in the endothelial cells, as described herein, by a variety of means. One such means is by contacting the endothelial cells with an effective amount of one or more agents that increase endogenous activation of ALK2. Such agents can be in purified form. Such agents include TGF-β2 (Shah et al. (1996) Cell 85:331-343; Lawrence DA, (1985) Biol Cell. 53(2):93-8; Roberts et al. (1985) Cancer Surv. 4(4):683-705; Madisen et al., (1988) DNA 7(1): 1-8) and BMP4 (Oida et al., (1995) Mitochondrial DNA 5: 273-275; Shore et al., (1998) Cal Tissue Int 63: 221-229), BMP2 and BMP7. Analogs, variants and functional fragments of TGF-β2, BMP4, BMP2 and/or BMP7 which retain the ability to activate ALK2 may also be used. Variants may be, for example, the otherwise wild type amino acid sequence having one or more modifications to improve biophysical properties and/or clinical performance, examples of which are provided for BMP4 in U.S. Patent Application 20080070837. Another such agent is a peptide mimic of TGF-β2, BMP4, BMP2 and/or BMP7 (U.S. Pat. No. 5,780,436). Other such agents can be antibodies which activate the TGF-β2, BMP4, BMP2 and/or BMP7 receptors. In one embodiment, the agent(s) used is substantially pure. In one embodiment, the agent(s) used is derived from the same animal source as the endothelial cells to which it is contacted (e.g., human TGF-β2, BMP4, BMP2 and/or BMP7 is used for human endothelial cells), however, certain cross species activation is expected as well.

An effective amount is an amount that is sufficient to activate ALK2 of the cells to thereby induce the EndMT in a significant amount of the cells (e.g., a significant amount of a population of endothelial cells to which the agent is administered). This can be determined, for example, by monitoring ALK2 activation (e.g., phosphorylation and/or other cellular responses such as activation of downstream signaling molecules) in the population of cells. For example, the cells can be monitored for a significant decrease in expression of VE-cadherein and/or a significant increase in expression of one or more of STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, CD124, CD248, CD133, c-kit, CD105, TAZ, TIE-2, and FSP-1, as evidence of EndMT. Such expression can be determined by a variety of methods, such as detection of protein expression levels by immunobased detection (e.g., Western Blot analysis, immunoprecipitation, flow cytometry) or by detection of mRNA levels (e.g., Northern Blot analysis, RT-PCR, etc). Detection of sufficient ALK2 activation is also indicated by EndMT, which is detected by monitoring one or more aspects of the cellular phenotype. For example, the expression of TIE-2 and/or FSP-1 is one such indicator. Another such indicator is morphology of the cells, which adopt a typical fibroblast-like morphology upon EndMT.

In one embodiment, the agent TGF-β2, BMP4, BMP2 and/or BMP7 is contacted to the cells at a concentration of about 10 ng/ml. Other useful concentrations include, without limitation, about 1, about 2, about 3, about 4, or about 5 ng/ml. Alternatively, a concentration of about 6, about 7, about 8, or about 9 ng/ml can be used. Also, a concentration of about 11, about 12, about 13, . . . about 19, or about 20 ng/ml can be used. An effective amount of the agents can be determined by the skilled practitioner.

Contacting the cells with about 10 ng/ml TGF-β2, BMP4, BMP2 and/or BMP7 for about 48 hours activates the ALK2 for a duration sufficient to induce of EndMT. Contacting the cells for less time (e.g. at least about 12 hours, at least about 24 hours, and at least 36 hours) is also expected to produce EndMT in a significant amount of cells. In one embodiment, contact is for less than 12 hours, for example about 11, about 10, about 9, . . . about 2, or about 1 hour. Contacting the cells for greater than about 48 hours is also considered useful in the instant invention. The times of exposure and the concentration of exposure can be determined for the specific agent used, for the specific endothelial cells population, by the skilled practitioner. In one embodiment, the serum starvation is continued throughout at least part of the duration of exposure to the agent. In one embodiment, the serum starvation is through the entire duration of exposure to the agent. Other methods of activation of ALK2, for these recited durations, are also envisioned.

### BMP4 and TGF-β2

BMP4 is synthesized as an inactive 50-kDa precursor protein, which dimerizes within cells via an intermolecular disulfide bond. The inactive BMP4 precursor is cleaved by members of the subtilisin-like proprotein convertase family into an active carboxyl-terminal mature BMP4 protein dimer (25 kDa for the monomer). The active mature form is secreted. Bone morphogenetic protein 4 (BMP4) belongs to the TGF-β superfamily of proteins. BMP-4 and BMP-7 are each 98% conserved between human and mouse. The DNA sequence of BMP4 is listed at GenBank accession number AF035427. Human BMP-4 shares 85% aa sequence identity with human BMP-2 and less than 50% aa sequence identity with other BMPs. Human BMP-7 shares approximately 60-70% aa sequence identity with BMP-5, -6, and -8 and less than 50% aa sequence identity with other BMPs. Recombinant Human BMP-4 is a homodimeric protein consisting of two 116 amino acid chains. The predicted molecular weight of each monomer is Mr 13 kDa. The monomer is glycosylated. The biological activity of human BMP-4 can be determined, for example, by its ability to induce alkaline phosphatase production by mouse ATDC5 chondrogenic cells.

TGF-β2 is a member of a family for which there are at least five forms (TGF-β1, TGF-β2, TGF-β3, TGF-β4, and TGF-β5). TGF-β2 has a precursor form of 414 amino acids and is also processed to a homodimer from the carboxy-terminal 112 amino acids that shares approximately 70% homology with the active form of TGF-β1 (Marquardt et al., J. Biol. Chem. 262: 12127 (1987)). TGF-β2 has been purified from porcine platelets (Seyedin et al., J. Biol. Chem., 262: 1946-1949 (1987)) and human glioblastoma cells (Wrann et al., EMBO J., 6: 1633 (1987)), and recombinant human TGF-β2 has been cloned (deMartin et al., EMBO J. 6: 3673 (1987); U.S. Pat. Nos. 4,774,322; 4,843,063; and 4,848,063 regarding CIF-A and CIF-B, now recognized as TGF-beta1 and 2, respectively). Generation of functional recombinant TGF-β2 is described in the art (Caltabiano et al., Gene 85: 479-488 (1989)). See Ellingsworth et al., J. Biol. Chem., 261: 12362-12367 (1986).

The recombinant production of TGF-β1, TGF-β2, and TGF-133 is described in U.S. Pat. Nos. 5,061,786; 5,268,455 and 5,801,231. See also U.S. Pat. No. 5,120,535 on a TGF-β2 used for treating hormonally responsive carcinoma and for production of antibodies. The heterodimer of TGF-β1 and TGF-β2, called TGF-β1.2, has been identified and its uses demonstrated, as disclosed in U.S. Pat. Nos. 4,931,548 and 5,304,541, the latter also disclosing an antibody thereto. WO 1990/00900, filed 20 Jul. 1989, discloses treatment of inflammatory disorders with homodimeric TGF-β1 and -β2, and the heterodimer TGF-β1.2. U.S. Pat. No. 5,462,925 discloses a heterodimer of TGF-β2 and TGF-133. A refined 3-dimensional crystal structure of TGF-β2 described, by Daopin et al., Proteins, 17, pp. 176-192 (1993).

Suitable methods are known for purifying TGF-β molecules from various species such as human, mouse, green monkey, pig, bovine, chick, and frog, and from various body sources such as bone, platelets, or placenta, for producing it in recombinant cell culture, and for determining its activity. See, for example, Derynck et al., Nature, 316: 701-705 (1985); European Pat. Pub. Nos. 200,341 published Dec. 10, 1986, 169,016 published Jan. 22, 1986, 268,561 published May 25, 1988, and 267,463 published May 18, 1988; U.S. Pat. No. 4,774,322; Cheifetz et al, Cell, 48: 409-415 (1987); Jakowlew et al., Molecular Endocrin., 2: 747-755 (1988); Dijke et al., Proc. Natl. Acad. Sci. (U.S.A.), 85: 4715-4719 (1988); Derynck et al., J. Biol. Chem. 261: 43774379 (1986); Sharples et al., DNA, 6: 239-244 (1987); Derynck et al., Nucl. Acids. Res., 15: 3188-3189 (1987); Deryncketal., Nucl. Acids. Res., 15: 3187 (1987); Derynck et al., EMBO J., 7: 3737-3743 (1988)); Seyedin et al., J. Biol. Chem. 261: 5693-5695 (1986); Madisen et al., DNA 7: 1-8 (1988); and Hanks et al., Proc. Natl. Acad. Sci. (U.S.A.), 85: 79-82 (1988).

### BMP2 and BMP7

BMP2 and BMP7 are osteogenic bone morphogenetic proteins: they have been demonstrated to potently induce osteoblast differentiation in a variety of cell types (Cheng et al., J. Bone Joint Surg. 85-A: 1544-1552, (2003); Chen et al., Growth Factors 22 (4): 233-41 (2004); Marie et al., Histol. Histopathol. 17 (3): 877-85 (2002)). BMP7 induces the phosphorylation of SMAD1 and SMAD5, which in turn induce transcription of numerous osteogenic genes (Itoh et al., Embo J. 20 (15): 4132-42 (2001)). The recombinant production of BMP2 and BMP7 are provided in U.S. Patent Application 20090202638. The DNA sequence and encoded protein sequence of BMP2 is provided in NCBI accession NM_{—}001200. The DNA sequence and encoded protein sequence of BMP7 is provided in NCBI accession NM_{—}001719.

Another aspect of the invention relates to the multipotent mesenchymal stem-like cells or a population thereof. In one embodiment, the multipotent mesenchymal stem-like cells are produced in vitro by the methods described herein. In one embodiment, the mesenchymal stem-like cells express characteristic levels of TIE-2 and FSP-1. In one embodiment, the mesenchymal stem-like cells exhibit a fibroblast-like morphology.

Mesenchymal stem cell(s) are capable of self renewal or differentiation into any particular lineage within the mesodermal germ layer. Mesenchymal stem cells have the ability to commit within the mesodermal lineage from a single cell any time during their life-span. This commitment process results from the use of general or specific mesodermal lineage-commitment agents. Mesenchymal stem cells may form any cell type within the mesodermal lineage, including, but not limited to, bone, skeletal muscle, smooth muscle, cardiac muscle, white fat, brown fat, connective tissues, connective tissue septae, loose areolar connective tissue, fibrous organ capsules, tendons, ligaments, dermis, hyaline cartilage, elastic cartilage fibrocartilage, articular cartilage, growth plate cartilage, endothelial cells, meninges, periosteum, perichondrium, osteoclasts, chondroclasts, and neural cells. The mesenchymal stem-like cells described herein likewise have the ability to form any of these cells types by exposure to the appropriate mesodermal lineage-commitment agents and/or culture conditions.

Evidence indicates that the multipotent mesenchymal stem-like cells possess the same potential for differentiation as mesenchymal stem cells. As such, the present invention relates to methods for generating any such cells from the multipotent mesenchymal stem-like cells. Such cells include, without limitation, osteoblast-like cells, chondrocyte-like cells, adipocyte-like cells, myocyte-like cells, tendonocyte-like cells, stromocyte-like cells, and neural-like cells. These cells are produced by incubation of the multipotent mesenchmal stem-like cells described herein, under the appropriate differentiation culture conditions for differentiation into the desired cell type. One such culture condition is incubation in the appropriate differentiation culture medium (e.g., osteogenic, chondrogenic, adipogenic, neuralgenic, myogenic, or cardiomyogenic culture medium). Such media are known in the art. Some examples of specific culture conditions useful in the invention are discussed herein, and/or presented in the Examples section below. Incubation is for an amount of time sufficient to produce the desired differentiation on a significant amount of the population of cells being incubated.

Examples of chondrogenic culture conditions are described in Hildebrandt et al., (Annals of Anatomy—Anatomischer Anzeiger 191(1): 23-32 (2009)). In one embodiment, the chondrogenic culture medium contains dexamethasone and/or BMP2. Examples of chondrogenic culture conditions are described in Tew et al., (Methods 45(1): 2-9 (2008)), Mackay et al., (Tissue Engineering 4(4): 415-428 (1998)), and Tan et al., (Cell Tissue Banking; Human amnion as a novel cell delivery vehicle for chondrogenic mesenchymal stem cells; ISSN 1573-6814 (Online) (2009)). In one embodiment, the chondrogenic culture medium contains dexamethasone and/or TGFβ3.

Examples of myogenic culture conditions are described in Gornostaeva et al., (Bulletin of Experimental Biology and Medicine 141: 493-499 (2006)), Dang et al., (Adv Mater Deerfield 19(19): 2775-2779 (2007)) and Gang et al., (Stem Cells 22(4): 617-624 (2004)).

Examples of adipogenic culture conditions are described in U.S. Pat. No. 5,827,740. In one embodiment, the adipogenic culture medium contains glucocorticoid and/or an agent that increases the levels of cyclic AMP in a cell. The adipocyte-like cells can be cultured in a variety of ways, and with a variety of materials, to form an appropriate composition for use in reconstructive and cosmetic surgery. In one non-limiting example, the cells may be combined with a biomatrix to form a two dimensional or three dimensional material as needed. The mesenchymal-stem like cells described herein can be mixed with a biocompatible material such as collagen, collagen sponge, alginate, polylactic acid material etc. to form a composite. The composite would then be treated to induce adipogenic differentiation of the cells in vitro for 1-3 weeks, then implanted when needed. For example, adipogenic cells could be mixed with a solubilized collagen or other biomaterial which is then allowed to gel to form a three dimensional composite that could be used for breast augmentation following mastectomy. Such a composite could be formed or sculpted to the appropriate size and shape. Another composition includes the culturing of mesenchymal stem-like cells on the acellular skin matrix that is currently on the market such as the product by LifeCell Corporation. In this format the cells would be cultured to populate the matrix and then caused to differentiate as described. The matrix with the adipogenic cells could then be cut by the surgeon to fit the site of reconstruction. As an alternative mesencymal-stem like cells could be induced to become adipocytes prior to their introduction into the biocompatible materials. As another alternative, mesenchymal stem-like cells in combination with compounds which promote differentiation into adipocytes may be used with a biomatrix as described without culturing for a period of time to induce differentiation whereby differentiation is induced in whole or in part in vivo.

Examples of neurogenic culture conditions are described in Jiang, et al., (Nature 418: 41-49 (2002)). Lee et al., (Stem Cells 23(7): 1012-20 (2005)), Cho et al., (Mol. BioSyst. 5: 609-611 (2009)) and Shakhbazov et al., (Bulletin of Experimental Biology and Medicine 147: 513-516 (2009)). Such culture conditions include, without limitation, culture on an appropriately coated substrate (e.g., plated on fibronectin-coated polystyrene wells in the neurogenic medium).

Differentiated cells are identified as differentiated, for example, by the presence (e.g., expression) of one or more cell type-specific markers, by cellular morphology, by the ability to form a particular cell type (e.g., adipocyte, myocyte, cardiomyocyte, chondrocyte, osteoblast, neuron), or combinations thereof. Those skilled in the art can readily determine the percentage of differentiated cells in a population using various well-known methods, such as fluorescence activated cell sorting (FACS). Ranges of purity in populations generated by the methods described herein comprising differentiated cells are about 50 to about 55%, about 55% to about 60%, and about 65% to about 70%. In some embodiments, the purity is about 70% to about 75%, about 75% to about 80%, about 80 to about 85%; and further may also be about 85% to about 90%, about 90%, to about 95%, and about 95% to about 100%. Purity of the population of cells or their progenitors can be determined, for example, according to the marker profile within a population. Dosages can be readily adjusted by those skilled in the art to obtained the desired or optimal purity (e.g., a decrease in purity may require an increase in dosage).

Another aspect of the present invention relates to cells generated from the multipotent mesenchymal stem-like cells, described herein. Evidence indicates that the multipotent mesenchymal stem-like cells have similar potential to differentiate as do mesenchymal stem cells. The present invention is intended to encompass any kind of cell, or population thereof, generated or otherwise arising from the multipotent-mesenchymal stem-like cells that correlates with a cell differentiated from a mesenchymal stem cell. As such, the present invention relates to any such cells generated from the multipotent-mesenchymal stem-like cells. Such cells include, without limitation, osteoblast-like cells, chondrocyte-like cells, adipocyte-like cells, myocyte-like cells, cardiomyocyte-like cells, and neural-like cells, and other cells described herein. Cells and populations thereof, which are further differentiated into more specialized cell types are also encompassed by the present invention. In one embodiment, the generated cells express characteristic amounts of TIE-2. The differentiated cells are determined by observation of phenotype, such as by detection of the presence of one or more cell type-specific markers, and/or morphology and/or the ability to differentiate further into a specific cell type and/or by the ability to perform a specific function. Specific cell type-specific markers for the various cell types are known in the art and routinely detected by conventional means. For example, osteoblast-like cells express the osteoblastic markers osteocalcin and osterix. Chondrocyte-like cells express the chondrotyte marker SOX9. Adipocyte-like cells express the adipocyte marker PPARγ2. Skeletal muscle markers such as myogenin and MyoD, and cardiotroponin I and slow muscle myosin can be used to identify cells that have differentiated along a myocyte-like lineage (e.g., cardiac troponin I for cardiomyocyte-like cells). Neronal markers such as neurofilament-L, neuron-specific enolase, neurofilament 200 and neuron-specific beta III-tubulin, can be used to identify cells that have differentiated along a neural-like lineage. In one embodiment, the cells produced from the mesenchymal stem-like cells retain the expression of TIE-2.

A number of molecules that are specific markers of adipocytes have been described in the literature that will be useful to identify the adipocyte-like cells described herein. These include enzymes involved in the interconversion of fatty acids to triglycerides such as stearoyl-CoA-desaturase (SCDI) or the insulin responsive glucose transporter (GLUT4). The product of the ob gene, leptin is a 16,000 molecular weight polypeptide that is only expressed in pre-adipose cells or adipose tissue. The expression of CCAAT enhancer binding protein, C/EBP, has been shown to precede the expression of several markers of adipogenic differentiation and it is thought to play a key role in adipocyte development. Another marker is 422 adipose P2 (422/aP2), a protein whose expression is enhanced during adipocyte differentiation (Cheneval, et al, 1991). Lipid soluble dyes can also be used as markers of adipocyte differentiation. Lipid soluble dyes are available to stain lipid vaculoes in adipocytes. These include Nile Red, Nile Blue, Sudan Black and Oil Red O, among others. Each of these hydrophobic dyes has a propensity to accumulate in the lipid containing vaculoes of the developing adipocytes and can readily identify the adipogenic cells in populations of differentiating MSCS. At least one of these dyes can be used to isolate adipocytes from non-differentiated cells using a fluorescence activated cell sorter (FACS) (U.S. Pat. No. 5,827,740).

Chondrocytes (cartilage cells) are cells that are capable of expressing characteristic biochemical markers of chondrocytes, including but not limited to collagen type II, chondroitin sulfate, keratin sulfate and characteristic morphologic markers of smooth muscle, including but not limited to the rounded morphology observed in culture, and able to secrete collagen type II, including but not limited to the generation of tissue or matrices with hemodynamic properties of cartilage in vitro. Such markers are useful in identification of chondrocyte-like cells of the present invention.

Another aspect of the present invention relates to isolated multipotent mesenchymal stem-like cells generated in vivo or otherwise obtained from an in vivo source. Such cells can be obtained, for example, by obtaining a tissue or cell sample isolated from a subject, likely to contain such cells (e.g., endothelial cells from large and small blood vessels, described herein) and identifying and selecting for multipotent mesenchymal stem-like cells within the obtained sample. Such selection can be, for example, on the basis of expressed proteins, described herein. Useful methods of identifying and selecting such cells include, without limitation, immunological based methods, such as FACS. Once obtained, these cells can be further used in the methods described herein. Similarly, cells that are generated from multipotent mesenchymal stem-like cells (e.g., osteoblast-like cells, chondrocyte-like cells, adipocyte-like cells, neural like-cells, myocyte-like cells, and cells differentiated therefrom) can also be obtained from an in vivo source. This is performed by obtaining a tissue or cell sample isolated from a subject, likely to contain such cells (e.g., endothelial cells from large and small blood vessels, described herein) and identifying and selecting for the specific cells type(s) desired within the obtained sample. Such selection can be, for example, on the basis of expressed proteins, described herein.

In one embodiment, the cells of the present invention, described herein, have a normal karyotype.

The cells of the present invention can be used for diagnostic purposes, such as to diagnose an individual wherein that diagnosis requires a specific cell type of the individual. Rather than performing an invasive extraction (e.g., a biopsy) the skilled practitioner may instead generate a cell type that requires further characterization by generating them from cells isolated from the individual. For example, multipotent mesenchymal stem-like cells from endothelial cells from the individual (or isolated multipotent mesenchymal stem-like cells directly from the individual) can be induced to differentiate into the appropriate cell type for further characterization in the diagnosis, such as a bone cell. This would preclude the need for a painful and invasive bone biopsy. As such, another aspect of the present invention relates to a method for diagnosing a subject. In one embodiment, the method comprises isolating endothelial cells from the subject in need of diagnosis. The cells are then induced to transform into the desired cell type by the methods described herein.

The cells may then be characterized by methods appropriate for the diagnostic purposes. For example, the cells can be characterized for gene expression by analysis of their nucleic acid expression for one or more genes of interest. This can be done, for example, by determining the levels of mRNA transcribed from a gene(s) of interest (e.g., by northern blot analysis, PCR, etc.). Another example is characterization of the cells for protein expression of one or more proteins of interest. Protein expression can be determined qualitatively and/or quantitatively, for example, using immunodetection methods for a specific protein (e.g., Western blot analysis, immunoprecipitation, fluorescent activated cell sorting, etc.). The cells may also be characterized on the basis of their response to exposure to one or more agents of interest (e.g., a drug or toxin). Characterization of the cellular response to a drug can be used to determine an appropriate treatment regimen for the individual from whom the cells are obtained. As such, another aspect of the present invention relates to a method for drug testing for an individual. The method comprises obtaining multipotent mesenchymal stem-like cells from the individual, by the methods described herein, and inducing those cells to differentiate into one or more cell type described herein, and performing drug testing on those cells, to thereby determine the likelihood of pharmacological efficacy of the drug on the individual in a treatment regimen. An indication of non-responsiveness of the tested cells, compared to an appropriate control, would indicate low or no pharmacological efficacy of the drug on the individual. As such, it would indicate that the individual is unlikely to be responsive to a treatment regimen using that drug. An indication of responsiveness of the tested cells to the drug, compared to an appropriate control, An indication of non-responsiveness of the tested cells, compared to an appropriate control, would indicate pharmacological efficacy of the drug on the individual. Such a result would indicate a likelihood of the individual to be responsive to a treatment regimen using that drug. Such drug testing can be used in methods of determining treatment of an individual with a disease.

The cells of the present invention may also be used in assays of toxicity. The use of the differentiated cells described herein may be preferred in certain assays of toxicity, as such cells more closely resemble the cell types present in the tissues and organs of an organism. These differentiated cells will be very useful in assays of toxicity performed in vitro, i.e., using cultured cells or suspensions of cells. Such in vitro assays examine the toxicity to cultured cells or suspended cells of compounds or compositions, e.g., chemical, pharmaceutical or biological compounds or compositions, or biological agents. In this context, a particular compound or composition may be considered toxic or likely toxic, if it shows a detrimental effect on the viability of cells or on one or more aspect of cellular metabolism or function. Typically, the viability of cells in vitro may be measured using colorimetric assays, such as, e.g., the MTT (or MTT derivative) assays or LDH leakage assays, or using fluorescence-based assays, such as, e.g., the Live/Dead assay, GyQuant cell proliferation assay, or Essays of apoptosis. Other assays may measure particular aspects of cellular metabolism or function. While the above are non-limiting examples, a person skilled in the art will be able to make an appropriate choice of assay of toxicity to use in combination with the differentiated cells provided by the present invention, and will be knowledgeable of the technical requirements to perform such assay. Accordingly, in an embodiment, the present invention provides a differentiated cell or cells for use in assays of toxicity. In another embodiment, the present invention provides a differentiated cell or cells of human or animal origin for use in assays of toxicity. The use of human cells in assays of toxicity will provide a relevant reference for the potential toxicity of chemical compounds, compositions, or agents on the cell types present in human or animal tissues or organs, respectively. Moreover, because such chemical compounds or compositions may also be comprised in a sample obtainable from the environment, in one embodiment the present invention also provides differentiated cells for use in assays of ecological toxicity.

Another aspect of the present invention relates to tissues generated from the cells described herein. Such tissues can be generated in vitro, in vivo, or ex vivo, by methods known in the art for generation of tissues from osteoblast, chondrocytes, adipocytes, myocytes, cardiomyocytes, and neural cells. Such tissues include, cartilage, muscle, bone, connective tissue, adipose tissue and neural tissue. In one embodiment, a significant percentage of the cells within the tissue generated express a characteristic amount of TIE-2.

Another aspect of the present invention relates to pharmaceutical compositions for use in therapeutic methods which comprise or are based upon the multipotent stem-like cells of the present invention, including lineage-uncommitted populations of cells, lineage-committed populations of cells, tissues and organs generated therefrom, along with a pharmaceutically acceptable carrier or media. The pharmaceutical compositions may further comprise proliferation factors or lineage commitment factors that act on or modulate the stem-like cells of the present invention and/or the cells, tissues and organs derived therefrom, along with a pharmaceutically acceptable carrier or media. The pharmaceutical compositions of proliferation factors or lineage commitment factors may further comprise the stem-like cells of the present invention, or cells, tissues or organs derived therefrom. The composition is formulated for administration to a subject in need thereof. Specific formulations will depend upon the method of administration. Suitable methods of administration are described herein.

One aspect of the invention relates to methods of treatment of diseases, disorders or injury in a subject by administration of the cells described herein.

One aspect of the invention relates to methods of treatment of diseases, disorders or injury in a subject by administration of the cells described herein. In one embodiment, the multipotent mesenchymal stem-like cells, or cells generated therefrom, are administered to a subject. The cells of this invention can be administered, for example, by injection, transplantation or surgical operation. Administration can be in the form of a pharmaceutical composition comprising the cells and a pharmaceutically acceptable carrier.

Diseases suitable for treatment include the use of myocyte-like cells for enhancement of muscle bulk; the use of cardiomyocyte-like cells (e.g., cardiomyocytes) for use in the treatment of cardiac diseases, such as, e.g., myocarditis, cardiomyopathy, heart failure, damage caused by heart attacks, hypertension, atherosclerosis, or heart valve dysfunction; the use of neuronal-like cells for the treatment of CNS disorders, such as, e.g., neurodegenerative disorders, including among others Alzheimer's disease, Parkinson's disease, muscular dystrophy, and Huntington's disease, or CNS damage, such as, e.g., resulting from stroke or spinal cord injury; the use of chondrocyte-like cells to treat diseases of the joints or cartilage, such as, e.g., cartilage tears, cartilage thinning, or osteoarthritis; the use of osteocyte-like cells to treat bone disorders, such as, e.g., bone fractures, non-healing fractures, or osteoarthritis.

In one embodiment, neuron-like cells are administered for the treatment of diseases of the nervous system. In one embodiment, the nervous system disease is neurodegenerative disease. Neurodegenerative disease refers to any condition characterized by the progressive loss of neurons, due to cell death, in the central nervous system of a subject. Such diseases include, without limitation, Parkinson's disease, muscular dystrophy, Huntington's disease, Alzheimer's disease, amyotrophic lateral sclerosis (ALS), multiple system atrophy, Lewy body dementia, peripheral sensory neuropathies or spinal cord injuries.

In one embodiment, the multipotent mesenchymal stem-like cells, or cells generated therefrom, are administered to a subject. Administration can be in the form of a pharmaceutical composition comprising the cells and a pharmaceutically acceptable carrier.

The mesenchyal stem-like cells or cells generated therefrom, can be administered to a subject directly to a tissue or organ of interest (e.g., by direct injection). In one embodiment, cells of the invention are provided to a site where an increase in the number of cells is desired, for example, due to disease, damage, injury, or excess cell death. Alternatively, cells of the invention can be provided indirectly to a tissue or organ of interest, for example, by administration into the circulatory system. If desired, the cells are delivered to a portion of the circulatory system that supplies the tissue or organ to be repaired or regenerated.

Advantageously, cells of the invention engraft within the tissue or organ. If desired, expansion and differentiation agents can be provided prior to, during or after administration of the cells to increase, maintain, or enhance production or differentiation of the cells in vivo. Compositions of the invention include pharmaceutical compositions comprising differentiated cells or their progenitors and a pharmaceutically acceptable carrier.

Methods for administering cells are known in the art, and include, but are not limited to, catheter administration, systemic injection, localized injection, intravenous injection, intramuscular, intracardiac injection or parenteral administration. When administering a therapeutic composition of the present invention (e.g., a pharmaceutical composition), it will generally be formulated in a unit dosage injectable form (solution, suspension, emulsion).

Administration can be autologous or heterologous. For example, cells obtained from one subject, can be administered to the same subject or a different, compatible subject.

Compositions of the invention (e.g., cells in a suitable vehicle) can be provided directly to a tissue or organ of interest, such as a tissue or organ having a deficiency in cell number as a result of injury or disease. Such tissues include, without limitation, bone, skeletal muscle, smooth muscle, cardiac muscle, white fat, brown fat, connective tissues, connective tissue septae, loose areolar connective tissue, fibrous organ capsules, tendons, ligaments, dermis, bone, hyaline cartilage, elastic cartilage fibrocartilage, articular cartilage, growth plate cartilage, endothelial cells, meninges, periosteum, perichondrium, osteoclast, chondroclast, and neural. Alternatively, compositions can be provided indirectly to the tissue or organ of interest, for example, by administration into the circulatory system. Compositions can be administered to subjects in need thereof by a variety of administration routes. Methods of administration, generally speaking, may be practiced using any mode of administration that is medically acceptable, meaning any mode that produces effective levels of the active compounds without causing clinically unacceptable adverse effects, many of which are described herein. Such modes of administration include intramuscular, intra-cardiac, oral, rectal, subcutaneous, topical, intraocular, buccal, intravaginal, intracisternal, intracerebroventricular, intratracheal, nasal, transdermal, within/on implants, e.g., fibers such as collagen, osmotic pumps, or grafts comprising differentiated cells, etc., or parenteral routes. A particular method of administration involves coating, embedding or derivatizing fibers, such as collagen fibers, protein polymers, etc. with therapeutic proteins. Other useful approaches are described in Otto, D. et al., (J Neurosci Res. 1989 January; 22(1):83-91) and in Otto, D. and Unsicker, K. (J. Neurosci. 1990 June; 10(6):1912-21).

In one approach, stem-like cells, or differentiated cells derived therefrom, obtained in vivo or generated in vitro by the methods described herein, are implanted into a host. The transplantation can be autologous, such that the donor of the cells is the recipient of the transplanted cells; or the transplantation can be heterologous, such that the donor of the cells is not the recipient of the transplanted cells. Once transferred into a host, the cells are engrafted, such that they assume the function and architecture of the native host tissue.

Stem-like cells and the differentiated cells derived therefrom, can be cultured, treated with agents and/or administered in the presence of polymer scaffolds. If desired, agents described herein are incorporated into the polymer scaffold to promote cell survival, proliferation, enhance maintenance of a cellular phenotype. Polymer scaffolds are designed to optimize gas, nutrient, and waste exchange by diffusion. Polymer scaffolds can comprise, for example, a porous, non-woven array of fibers. The polymer scaffold can be shaped to maximize surface area, to allow adequate diffusion of nutrients and growth factors to the cells. Taking these parameters into consideration, one of skill in the art could configure a polymer scaffold having sufficient surface area for the cells to be nourished by diffusion until new blood vessels interdigitate the implanted engineered-tissue using methods known in the art. Polymer scaffolds can comprise a fibrillar structure. The fibers can be round, scalloped, flattened, star-shaped, solitary or entwined with other fibers. Branching fibers can be used, increasing surface area proportionately to volume.

Unless otherwise specified, the term “polymer” includes polymers and monomers that can be polymerized or adhered to form an integral unit. The polymer can be non-biodegradable or biodegradable, typically via hydrolysis or enzymatic cleavage. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refers to cleavage of the polymer chain, such that the molecular weight stays approximately constant at the oligomer level and particles of polymer remain following degradation.

Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbornene, hydrogels, metallic alloys, and oligo(.epsilon.-caprolactone)diol as switching segment/oligo(p-dioxyanone)diol as physical crosslink. Other suitable polymers can be obtained by reference to The Polymer Handbook, 3rd edition (Wiley, N.Y., 1989).

If desired, cells of the invention are delivered in combination with (prior to, concurrent with, or following the delivery of) agents that increase survival, increase proliferation, enhance differentiation, and/or promote maintenance of a differentiated cellular phenotype. In vitro and ex vivo applications of the invention involve the culture of stem-like cells or their progenitors with a selected agent to achieve a desired result. Cultures of cells (from the same individual and from different individuals) can be treated with expansion agents prior to, during, or following differentiation to increase the number of differentiated cells. Stem-like cells can then be used for a variety of therapeutic applications (e.g., tissue or organ repair, regeneration, treatment of an ischemic tissue, or treatment of myocardial infarction). If desired, stem-like cells, or cells derived therefrom, of the invention, are delivered in combination with other factors that promote cell survival, differentiation, or engraftment. Such factors, include but are not limited to nutrients, growth factors, agents that induce differentiation, products of secretion, immunomodulators, inhibitors of inflammation, regression factors, hormones, or other biologically active compounds.

One consideration concerning the therapeutic use of differentiated cells of the invention or their progenitors is the quantity of cells necessary to achieve an optimal effect. In general, doses ranging from 1 to 4×10⁷ cells may be used. However, different scenarios may require optimization of the amount of cells injected into a tissue of interest. Thus, the quantity of cells to be administered will vary for the subject being treated. In a preferred embodiment, between about 10⁴ to about 10⁸, more preferably about 10⁵ to about 10⁷, and still more preferably, about 1, 2, 3, 4, 5, 6, or about 7×10⁷ stem-like cells of the invention can be administered to a human subject.

Fewer cells can be administered directly a tissue where an increase in cell number is desirable. Preferably, between about 10² to about 10⁶, more preferably about 10³ to about 10⁵, and still more preferably, about 10⁴ stem-like cells or their progenitors can be administered to a human subject. However, the precise determination of what would be considered an effective dose may be based on factors individual to each subject, including their size, age, sex, weight, and condition of the particular subject. As few as about 100-about 1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Typically, any additives (in addition to the active stem cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.
1. A method of producing multipotent cells from endothelial cells, comprising, activating ALK2 in the endothelial cells, in a serum starved environment, to thereby produce multipotent cells.
2. The method of paragraph 1, further comprising subjecting the endothelial cells to a threshold period of serum starvation prior to activating ALK2.
3. The method of paragraph 1 or 2, wherein activating ALK2 is by contacting the endothelial cells with TGFβ-2, and/or BMP4, and/or an analog, derivative or functional fragment thereof.
4. The method of paragraph 3, wherein the TGFβ-2, BMP4, and/or analog, derivative or functional fragment thereof is contacted to the endothelial cells at a concentration of about 10 ng/ml.
5. The method of paragraph 1-4, wherein activating ALK2 is for at least about 48 hours.
6. The method of paragraphs 2-5, wherein the threshold period of serum starvation is for at least about 24 hours.
7. The method of paragraphs 1-6, wherein the endothelial cells are selected from the group consisting of primate, equine, bovine, porcine, canine, feline, and rodent.
8. The method of paragraphs 1-7, wherein the endothelial cells are human.
9. The method of paragraphs 1-8, wherein the endothelial cells are primary vascular or primary microvascular endothelial cells.
10. The method of paragraph 1-9, wherein the endothelial cells are isolated.
11. The method of paragraphs 1-10, wherein the endothelial cells are primary human umbilical vein endothelial cells (HUVEC) or primary human cutaneous microvascular endothelial cells (HCMEC).
12. The method of paragraphs 1-11, wherein activation of ALK2 significantly decreases expression of VE-cadherein of the cells and/or to significantly increase expression of one or more of STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, CD124.
13. A method of producing osteoblast-like cells, comprising incubating multipotent cells produced by the method of paragraphs 1-12, in osteogenic culture medium for a period sufficient to induce differentiation.
14. A method of producing isolated chondrocyte-like cells, comprising incubating multipotent cells produced by the method of paragraphs 1-12, in chondrogenic culture medium for a period sufficient to induce differentiation.
15. A method of producing adipocyte-like cells, comprising incubating multipotent cells produced by the method of paragraphs 1-12, in adipogenic culture medium for a period sufficient to induce differentiation.
16. A method of producing neural-like cells, comprising incubating multipotent cells produced by the method of paragraphs 1-12, in neuralgenic culture medium for a period sufficient to induce differentiation.
17. A method of producing myocyte-like cells, comprising incubating multipotent cells produced by the method of paragraphs 1-12, in myogenic culture medium for a period sufficient to induce differentiation.
18. A method of producing cardiomyocyte-like cells, comprising incubating multipotent cells produced by the method of paragraphs 1-12, in cardiomyogenic culture medium for a period sufficient to induce differentiation.
19. An isolated multipotent human mesenchymal cell, or population thereof, wherein the multipotent human mesenchymal cell expresses transcripts for STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, or CD124, or combinations thereof, and has a normal karyotype.
20. An isolated multipotent human mesenchymal cell or population thereof that expresses transcripts for TIE-2 and FSP-1.
21. The isolated multipotent human mesenchymal cell of paragraph 19 or 20, that is produced by the method of paragraphs 1-11.
22. An isolated multipotent cell or population thereof, produced by the method of paragraphs 1-12, that expresses TIE-2 and FSP-1.
23. The isolated multipotent cell or population thereof, of paragraph 22 that has fibroblast-like morphology.
24. The isolated multipotent cell or population thereof, of paragraphs 22 or 23 that is human.
25. An isolated cell or population thereof, that expresses one or more osteoblast specific markers and TIE-2.
26. The isolated cell or population thereof, of paragraph 25, wherein the osteoblast specific marker is osteocalcin or osterix.
27. The isolated cell or population thereof, of paragraph 25 or 26, that is produced by the method of paragraph 12.
28. An isolated cell or population thereof, that expresses one or more chondrocyte specific markers and TIE-2.
29. The isolated cell or population thereof, of paragraph 28, wherein the chondrocyte specific marker is SOX9.
30. The isolated cell or population thereof, of paragraph 28 or 29 that is produced by the method of paragraph 14.
31. An isolated cell or population thereof, that expresses one or more adipocyte specific markers and TIE-2.
32. The isolated cell of or population thereof, paragraph 31, wherein the adipocyte specific marker is PPARγ2.
33. The isolated cell or population thereof, of paragraph 31 or 32 that is produced by the method of paragraph 15.
34. An isolated cell or population thereof, that expresses one or more neuronal specific markers and TIE-2.
35. The isolated cell of or population thereof, paragraph 34, wherein the neuronal specific marker is neurofilament-L, neuron-specific enolase, neurofilament 200 and/or neuron-specific beta III-tubulin.
36. The isolated cell or population thereof, of paragraph 34 or 35, that is produced by the method of paragraph 16.
37. An isolated cell or population thereof, that expresses one or more myocyte specific markers and TIE-2.
38. The isolated cell or population thereof, of paragraph 37, wherein the myocyte specific marker is myogenin, MyoD, and/or slow muscle myosin.
39. The isolated cell or population thereof, of paragraph 37 or 38, that is produced by the method of paragraph 17.
40. An isolated cell or population thereof, that expresses one or more cardiomyocyte specific markers and TIE-2.
41. The isolated cell or population thereof of paragraph 40, wherein the cardiomyocyte specific marker is cardiac troponin-1.
42. The isolated cell or population thereof, of paragraph 40 or 41, that is produced by the method of paragraph 18.
43. A tissue generated from the cell or population thereof of paragraphs 19-42.
44. The tissue of paragraph 43, that is selected from the group consisting of skeletal muscle, bone, cartilage, heart, connective tissue, adipose tissue, and neural tissue.

The invention is further illustrated by the following examples, which should not be construed as further limiting.

### EXAMPLES

### Example 1

The experiments detailed below show that chondrocytes and osteoblasts from FOP lesions express endothelial-specific markers suggesting that they are derived from vascular endothelial cells. Expressing the mutant ALK2 gene found in FOP patients in human endothelial cells causes EndMT and acquisition of a multipotent stem cell phenotype. Activation of ALK2 by recombinant TGF-β2 or BMP4 also promotes this mechanism, which is prevented by inhibitory ALK2-specific siRNA.

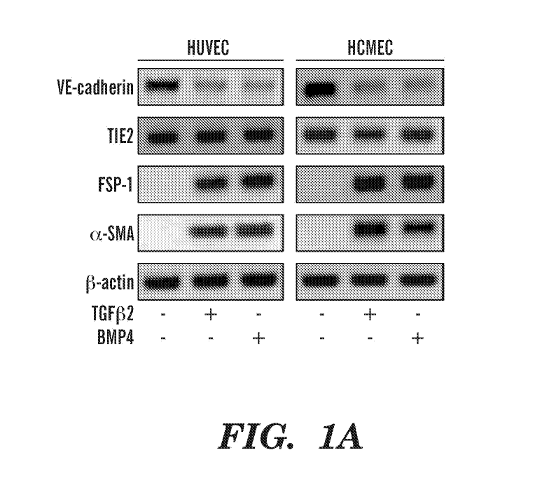

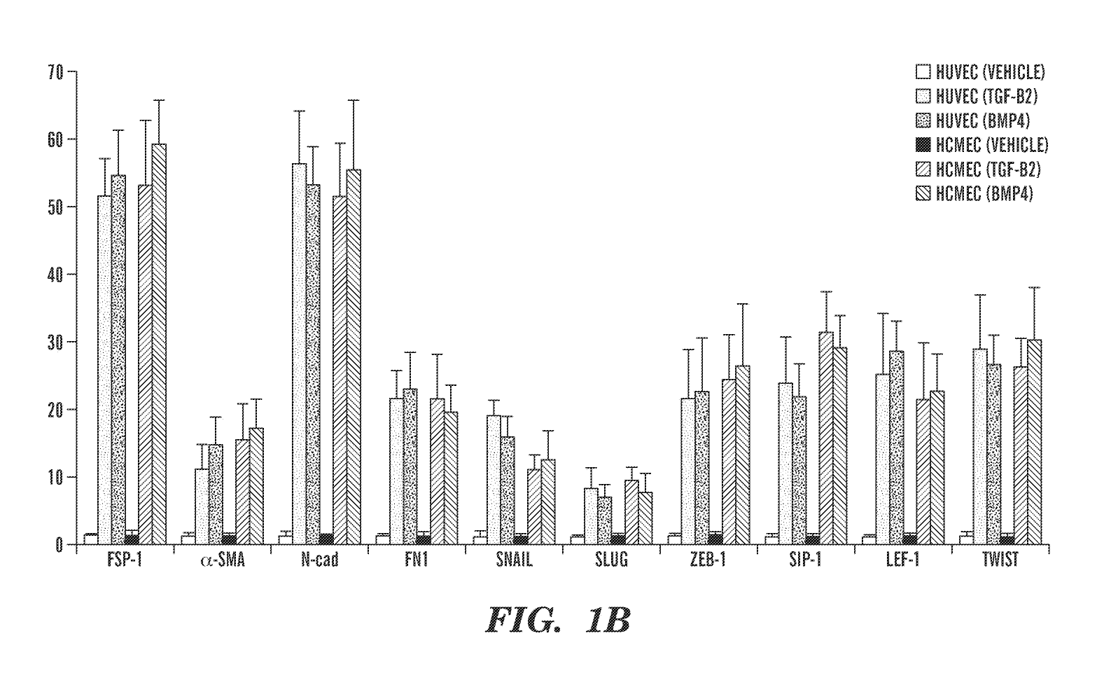

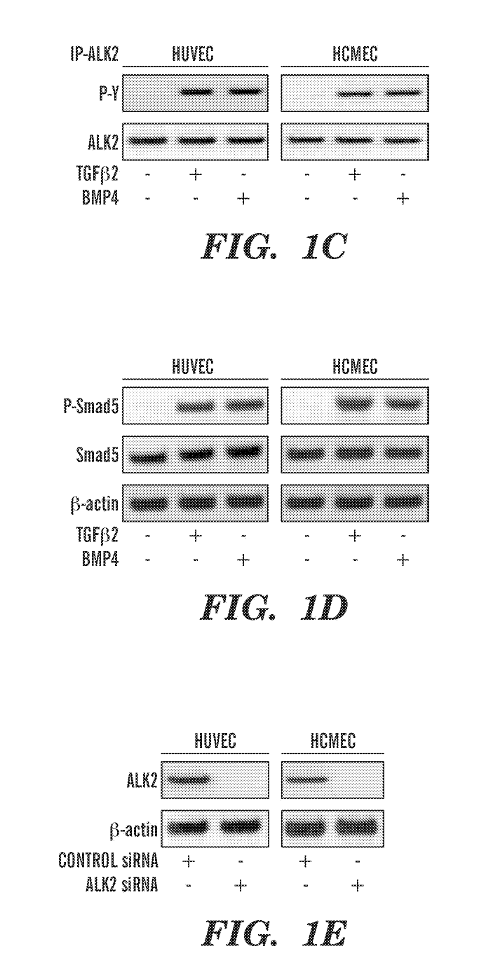

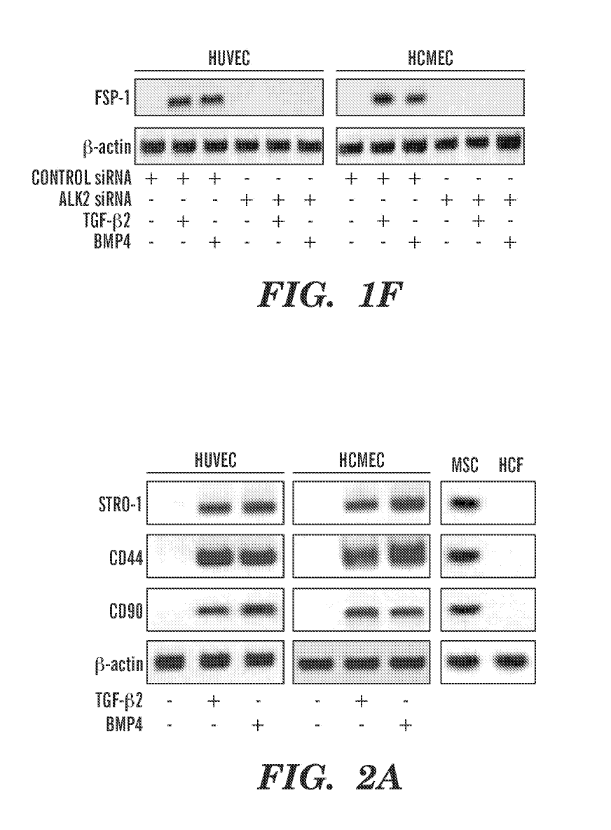

Human umbilical vein endothelial cells (HUVECs) and human cutaneous microvascular endothelial cells (HCMECs) were treated with recombinant TGF-β2 or BMP4 for 48 hours. Control cells were vehicle treated for 48 hours. DIC imaging of the treated cells revealed a change in cell morphology of endothelial cells treated with TGF-β2 or BMP4. Cells treated with TGF-β2 or BMP4 showed a distinct change from cobblestone-like endothelial cell morphology to fibroblast-like morphology. Immunocytochemistry and flow cytometry of treated cells indicated a change in co-expression of TIE2 and FSP-1 in endothelial cells treated with TGF-β2 or BMP4. Cells were co-stained using antibodies against the endothelial marker TIE2 and the mesenchymal marker FSP-1, then analyzed by fluorescence microscopy or flow cytometry. Vehicle treated cells were strongly positive for TIE2, but showed no staining for FSP-1, whereas TGF-β2 or BMP4 treated cells showed staining for both. To assess changes in protein expression of the endothelial markers VE-cadherin and TIE2, as well as the mesenchymal markers FSP-1 and α-SMA, lysates of cells treated under the same conditions were immunoblotted. VE-cadherin levels were significantly decreased in cells treated with TGF-β2 or BMP4, while FSP-1 and α-SMA levels increased. TIE-2 expression levels remained unchanged (FIG. 1A). Real-time quantitative PCR using RNA isolated from these cells to assess changes in expression of genes associated with EndMT^{1,24}, such as FSP-1, α-SMA, N-cadherin (N-cad), fibronectin (FN1), SNAI1 (Snail), SNAI2 (Slug), ZEB-1, SIP-1, LEF-1, and Twist, revealed that expression of these genes was much higher in cells treated with TGF-β2 or BMP4 than in control cells (FIG. 1B).

TGF-β2 and BMP4 associate with a common receptor known as ALK2^{25,26}. Phosphorylation of this receptor was therefore examined in cell lysates. Immunoprecipitation assays using antibodies against ALK2, followed by immunoblotting with phospho-tyrosine (P-Y) specific antibodies indicated that treatment of cells with TGF-β2 or BMP4 for 15 minutes induced receptor phosphorylation, whereas treatment with vehicle did not (FIG. 1C). Smad5, a downstream target of ALK2 signaling²⁵, was also phosphorylated after 1 hour of treatment with TGF-β2 or BMP4 (FIG. 1D).

To assess the importance of ALK2 in mediating EndMT, cells were transfected for 24 hours with a siRNA duplex specific for knockdown of ALK2. A scrambled non-specific siRNA duplex was used as a negative control. Immunoblotting of cell lysates showed complete inhibition of ALK2 expression by the ALK2-specific siRNA (FIG. 1E). Transfected cells were subsequently treated with vehicle, TGF-β2, or BMP4 for 48 hours, followed by lysis and immunoblotting for the mesenchymal marker FSP-1. TGF-β2 or BMP4 treated cells transfected with control siRNA had much higher expression of FSP-1 than vehicle-treated cells, whereas ALK2 siRNA treatment completely blocked these increases (FIG. 1F). β-actin was used as an internal control for all immunoblotting experiments. These results were confirmed by flow cytometry of cells stained in suspension with antibodies against the endothelial marker TIE2 and the mesenchymal marker FSP-1. Vehicle treated cells transfected with control siRNA or ALK2 siRNA showed no positive staining for FSP-1. TGF-β2 or BMP4 treated cells transfected with control siRNA showed highly increased numbers of cells expressing FSP-1, while cultures transfected with ALK2 siRNA showed very few cells expressing FSP-1.

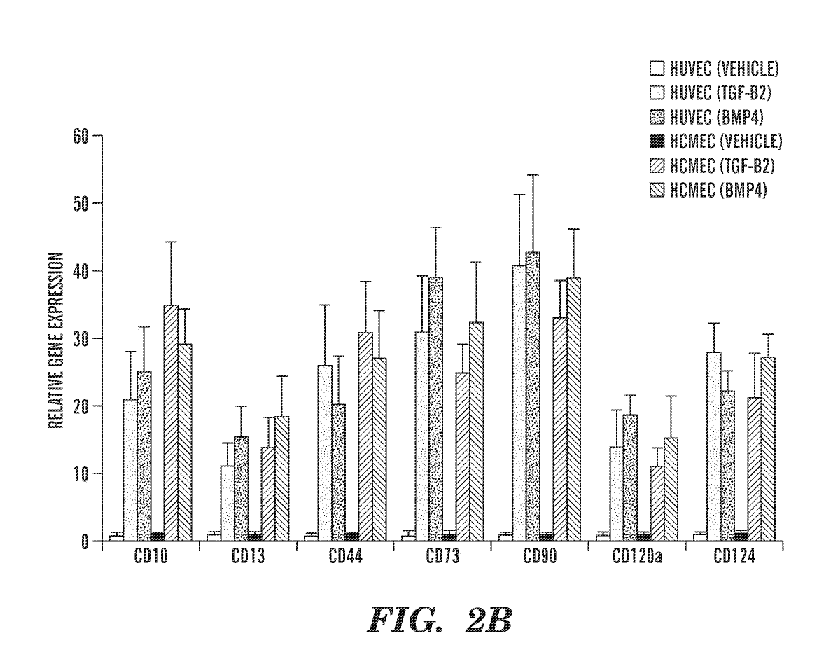

To investigate whether endothelial cells treated with TGF-β2 or BMP4 for 48 hours acquire a stem cell phenotype, the following experiments were performed. Immunocytochemistry and flow cytometry was performed on HUVEC and HCMEC to detect co-expression of TIE2 and STRO-1 in endothelial cells treated with TGF-β2 or BMP4. Immunofluorescence microscopy or flow cytometry of cells detected with antibodies against the endothelial marker TIE2 and the mesenchymal stem cell marker STRO-1 showed strong co-expression of the two proteins in cells treated with TGF-β2 or BMP4, but no STRO-1 expression in vehicle-treated cells. Immunoblotting of lysates collected from cells treated under the same experimental conditions showed that STRO-1 and other mesenchymal stem cell markers were not expressed in vehicle treated cells, but were strongly expressed in cells treated with TGF-β2 or BMP4. Lysates of bone marrow-derived mesenchymal stem cells showed positive expression of all of these proteins, but adult human corneal fibroblasts did not (FIG. 2A). Real-time quantitative PCR with RNA extracted from cells under the same experimental conditions showed that expression of genes normally expressed in mesenchymal stem cells²⁷, including CD10, CD13, CD44, CD73, CD90, CD120a, and CD124, was greatly increased in cells treated with TGF-β2 or BMP4 compared to control cells (FIG. 2B).

Since mesenchymal stem cells are multipotent, the differentiation capabilities of endothelial cells induced to undergo EndMT was examined. Endothelial cells (human bone marrow derived mesenchymal stem cells (MSC) and human corneal fibroblasts (HCF)) were treated with vehicle, TGF-β2, or BMP4 for 48 hours, followed by growth in osteogenic, chondrogenic or adipogenic culture medium. Both sets of cultures treated with TGF-β2 or BMP4 showed strong positive staining for alkaline phosphatase 7 days after osteogenic medium was added, indicating osteoblasts, whereas control cultures showed none. Furthermore, cells treated with TGF-β2 or BMP4 and grown in osteogenic medium for 21 days showed high levels of matrix calcification as indicated by alizarin red staining, further indicating the presence of osteoblasts. In contrast, vehicle treated cells showed no alizarin red staining. Similar results were found for both types of cells grown in chondrogenic medium for 14 days using the cartilage proteoglycan stain alcian blue to show chondrocytes, or in adipogenic medium for 7 days using oil red O staining to show adipocytes.

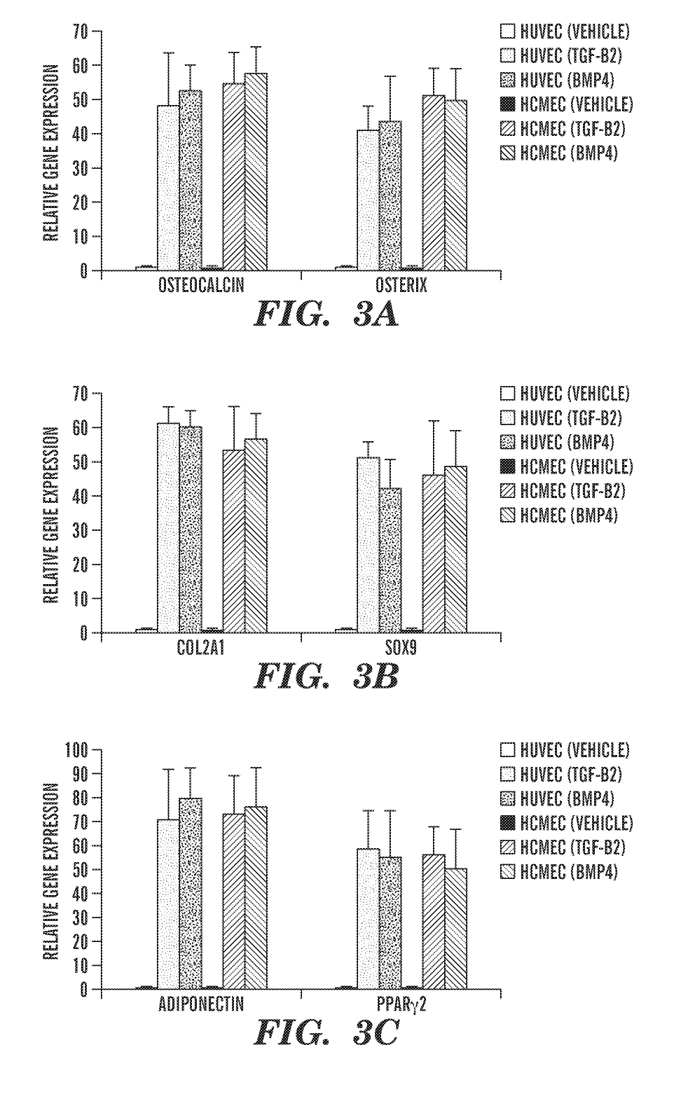

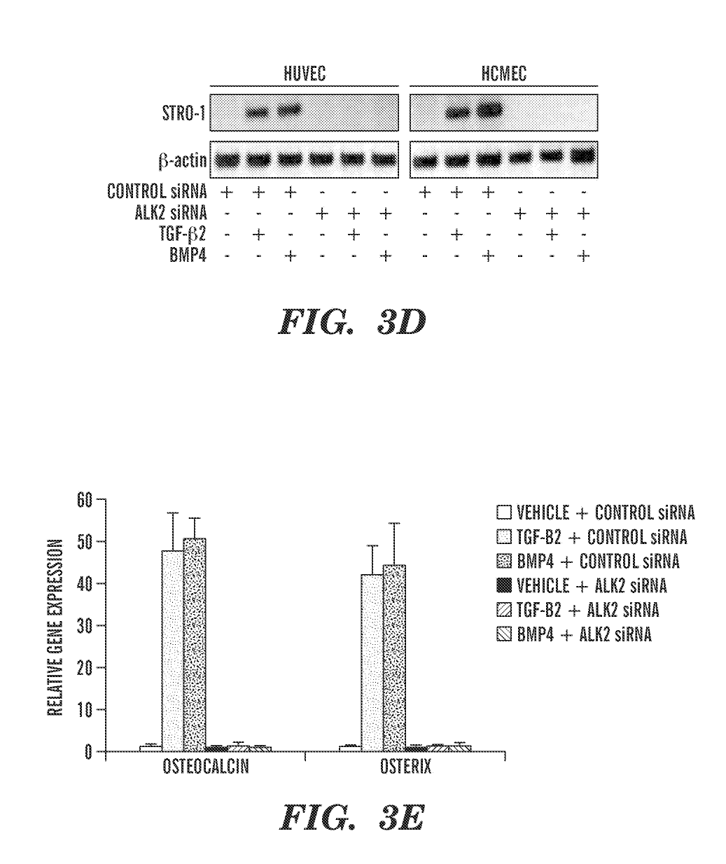

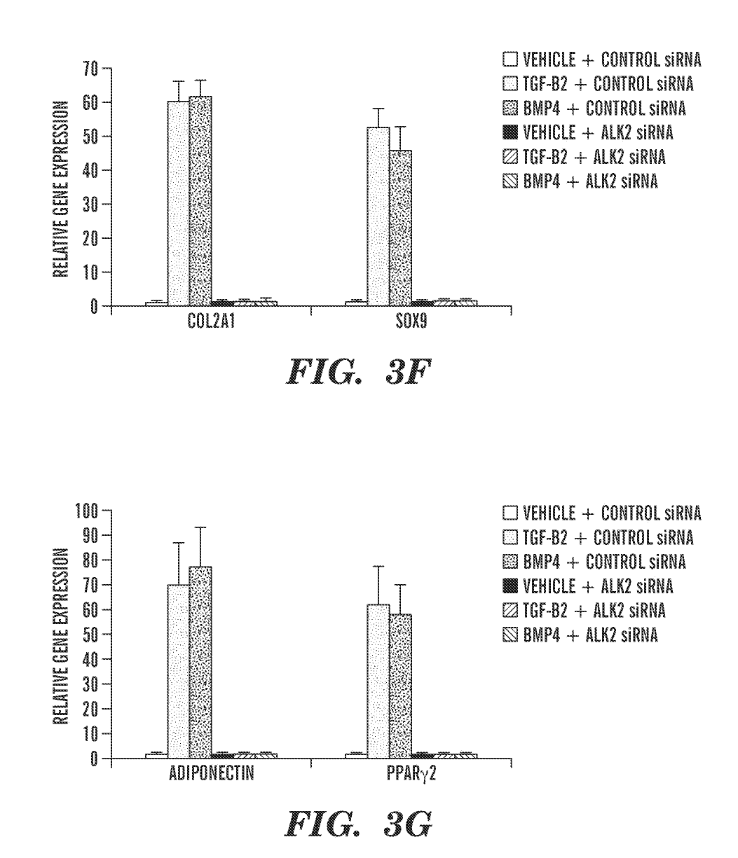

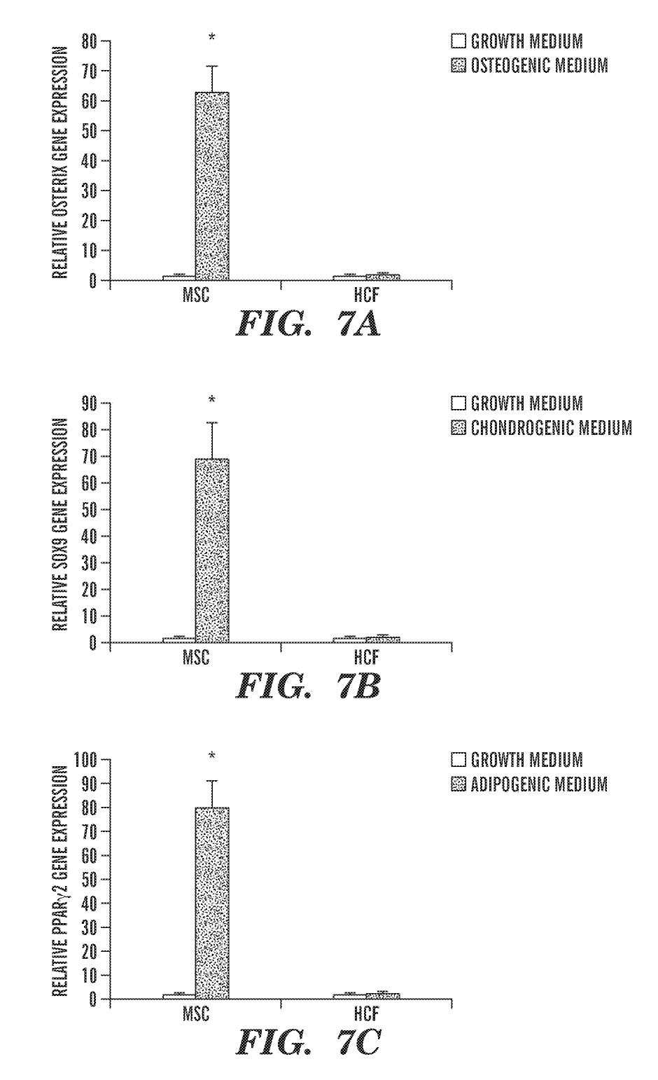

Real-time quantitative PCR using RNA extracted from endothelial cells treated with vehicle, TGF-β2 or BMP4 for 48 hours and then grown in osteogenic medium for 7 days, chondrogenic medium for 14 days or adipogenic medium for 7 days demonstrated dramatic increases in gene expression of the osteoblastic markers osteocalcin and osterix (FIG. 3A, 7A), the chondrocyte markers COL2A1 and SOX9 (FIG. 3B, 7B), and the adipocyte markers adiponectin and PPARγ2 (FIG. 3C, 7C) in cultures exposed to TGF-β2 or BMP4 compared to vehicle treated cells. Taken together, these data suggest that endothelial-derived mesenchymal stem cells can differentiate into osteoblasts, chondrocytes or adipocytes. The differentiation potential of these endothelial derived mesenchymal stem cells was found to be similar to that of bone marrow derived mesenchymal stem cells, whereas human corneal fibroblasts showed no differentiation potential (FIG. 7).

The siRNA expression knockdown experiments established that ALK2 is required for TGF-β2- or BMP4-induced EndMT (FIG. 1E-F), so whether ALK2 is also necessary for the multipotent stem cell phenotype was also investigated. Endothelial cells were transfected with control siRNA or ALK2 siRNA for 24 hours followed by treatment with vehicle, TGF-132 or BMP4 for 48 hours. Lysates were collected followed by immunoblotting with antibodies against the mesenchymal stem cell marker STRO-1. STRO-1 expression was undetectable in vehicle treated cells, but cells transfected with control siRNA and exposed to TGF-β2 or BMP4 showed high expression of STRO-1. ALK2 siRNA prevented TGF-β2- or BMP4-induced increases in this stem cell marker (FIG. 3D). When cells grown under the same experimental conditions were cultured in osteogenic medium for 7 days, TGF-β2 or BMP4 treated cultures that were transfected with control siRNA showed positive staining for alkaline phosphatase, whereas vehicle treated cultures showed none. Cells transfected with ALK2 siRNA also showed no positive staining indicating that expression of the ALK2 siRNA prevented differentiation of these cells. Similar results were found for matrix calcification by alizarin red staining after 21 days of incubation in osteogenic medium, chondrocyte proteoglycans by alcian blue staining after 14 days of incubation in chondrogenic medium, and adipocytes by oil red O staining after 7 days of incubation in adipogenic medium. Real-time quantitative PCR using RNA extracted from cells treated under the same conditions indicated that cells transfected with control siRNA and treated with TGF-β2 or BMP4 had dramatic increases in expression of osteocalcin and osterix after 7 days of incubation in osteogenic medium. Treatment with ALK2 siRNA prevented these increases (FIG. 3E). Similar expression patterns were found for COL2A1 and SOX9 in cultures incubated in chondrogenic medium for 14 days (FIG. 3F), as well as for adiponectin and PPARγ2 in cultures incubated in adipogenic medium for 7 days (FIG. 3G).

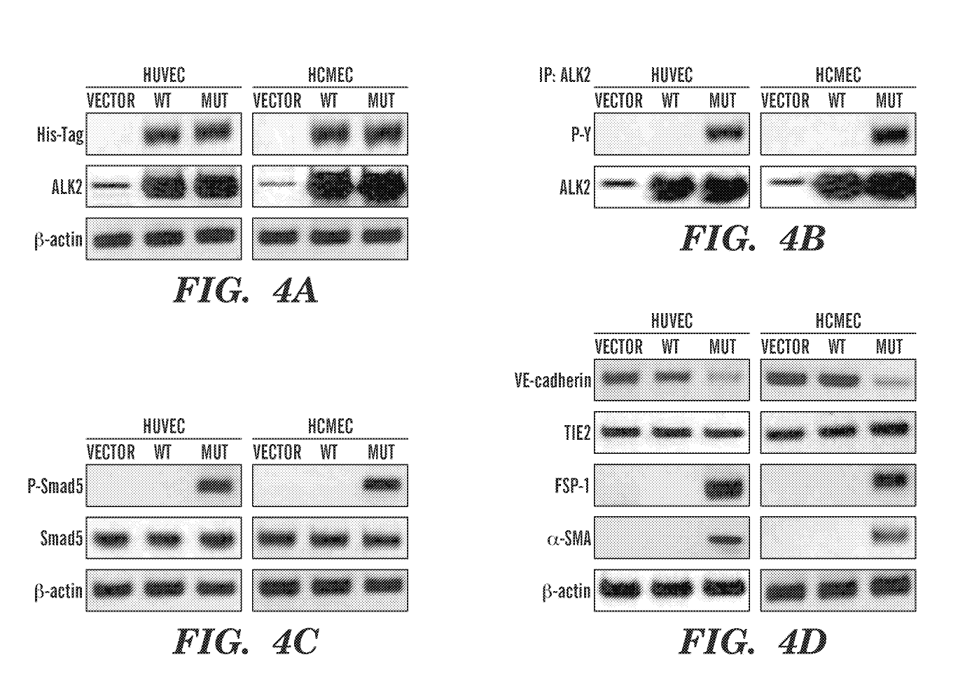

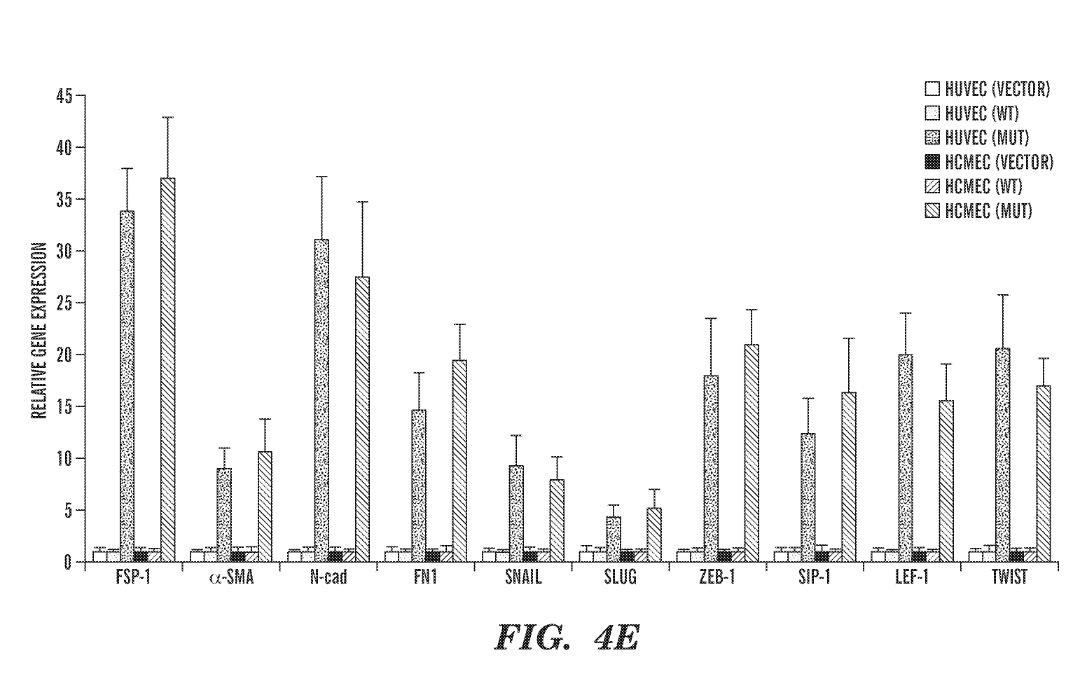

Infecting HUVECs and HCMECs with an adenoviral construct encoding wild-type and mutant (R206H) ALK2 resulted in greatly increased levels of ALK2 (FIG. 4A). Immunoblotting also showed that mutant ALK2 induced phosphorylation of ALK2 and Smad5, whereas infection with constructs encoding wild-type ALK2 or vector did not (FIG. 4B,C). EndMT was confirmed by immunoblotting showing that mutant ALK2 reduced VE-cadherin levels and increased FSP-1 and α-SMA expression. TIE2 expression remained constant (FIG. 4D). DIC imaging was used to investigate whether cell morphology in endothelial cells was changed by expression of mutant ALK2. Flow cytometry analysis was also used to investigate whether TIE2 and FSP-1 were co-expressed in cells containing the mutant ALK2 construct. Mutant ALK2 expression caused a change in endothelial cell shape to a mesenchymal morphology and induced co-expression of FSP-1 and TIE2. EndMT was further confirmed by real-time quantitative PCR showing that mutant ALK2 increased gene expression of the mesenchymal markers FSP-1, α-SMA, N-cadherin, fibronectin, Snail, Slug, ZEB-1, SIP-1, LEF-1, and Twist (FIG. 4E).

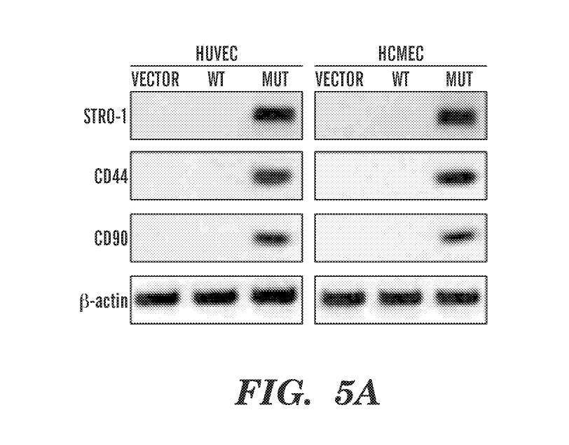

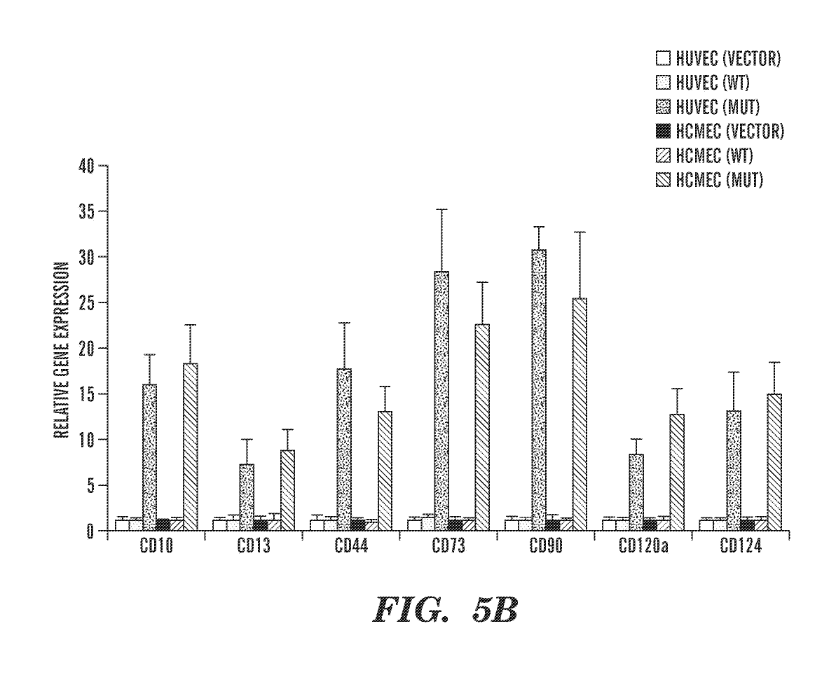

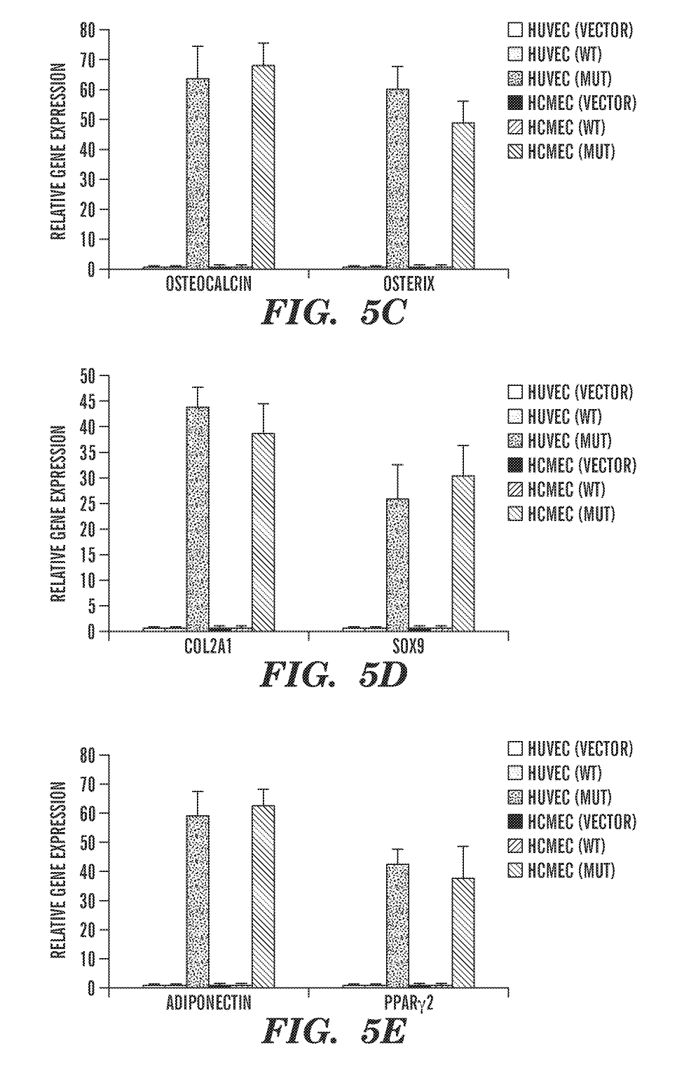

To determine if mutant ALK2 induces the stem cell phenotype, flow cytometry analysis for co-expression of TIE2 and STRO-1 was performed. Expression of mutant ALK2 was found to induce co-expression of TIE2 and STRO-1 in HUVEC and HCMEC, but wild-type ALK2 or the empty vector did not. This was further confirmed this by immunoblotting, showing that cells containing the mutant ALK2 construct expressed the stem cell markers STRO-1, CD44, and CD90 (FIG. 5A). Real-time PCR analysis showed that mutant ALK2 caused increased expression of genes expressed in mesenchymal stem cells including CD10, CD13, CD44, CD73, CD90, CD120a, and CD124 (FIG. 5B).

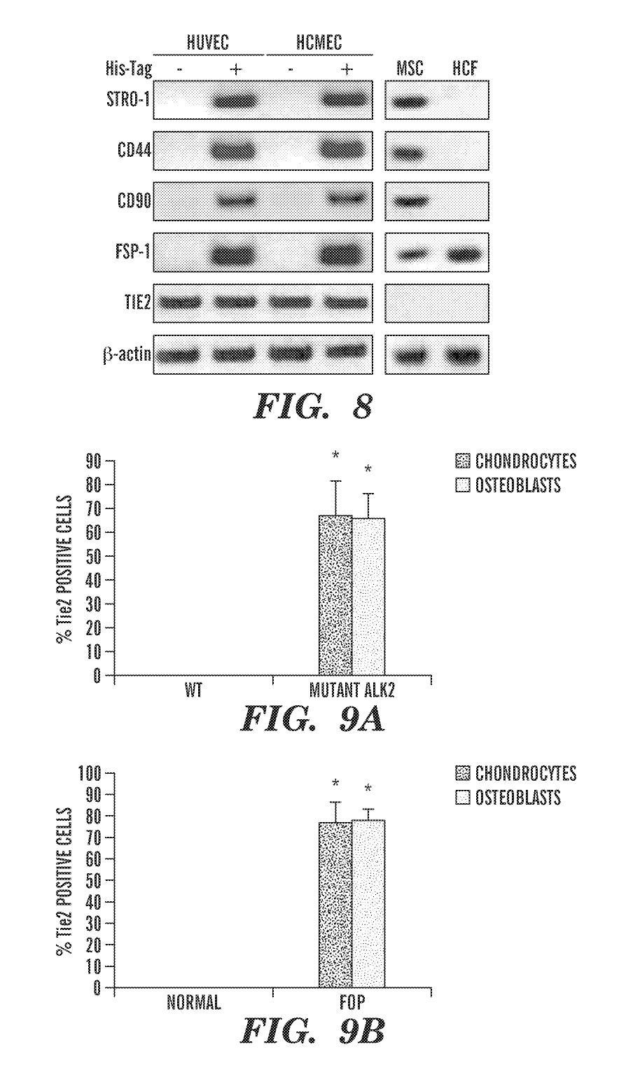

Endothelial cells were treated with adenoviral mutant ALK2 and stained with antibodies against the His-tagged protein. Fluorescence activated cell sorting was used to separate the mutant ALK2 (Mut) treated endothelial cells (HUVEC and HCMEC) that express the His-Tag (+) from those that do not (−). Immunoblotting of lysates from the separated cells showed that only the His-tag positive cell population expressed the mesenchymal (FSP-1) and stem cell (STRO-1, CD44, CD90) markers (FIG. 8).

Next, endothelial cells were exposed to the adenoviral expression constructs for 48 hours, followed by growth in osteogenic, chondrogenic, or adipogenic culture medium. Cultures were stained with alkaline phosphatase after 7 days in osteogenic medium, alizarin red after 21 days in osteogenic medium, alcian blue after 14 days in chondrogenic medium, or oil red O after 7 days in adipogenic medium. Positive staining of osteoblasts (alkaline phosphatase and alizarin red), chondrocytes (alcian blue), or adipocytes (oil red O) was performed in endothelial cell cultures (HCMEC and HUVEC)) treated with mutant ALK2 for 48 hours followed by growth in osteogenic, chondrogenic, or adipogenic culture medium. Endothelial cells (HCMEC and HUVEC) expressing mutant ALK2 differentiated into other cell types, whereas those treated with vector or wild-type constructs did not. These results were confirmed by real-time PCR analysis of osteoblast (osteocalcin, osterix), chondrocyte (COL2A1, SOX9), and adipocyte (adiponectin, PPARγ2) markers showing that mutant ALK2 increases expression of these genes when cells are grown in their respective differentiation medium (FIG. 5C-E).

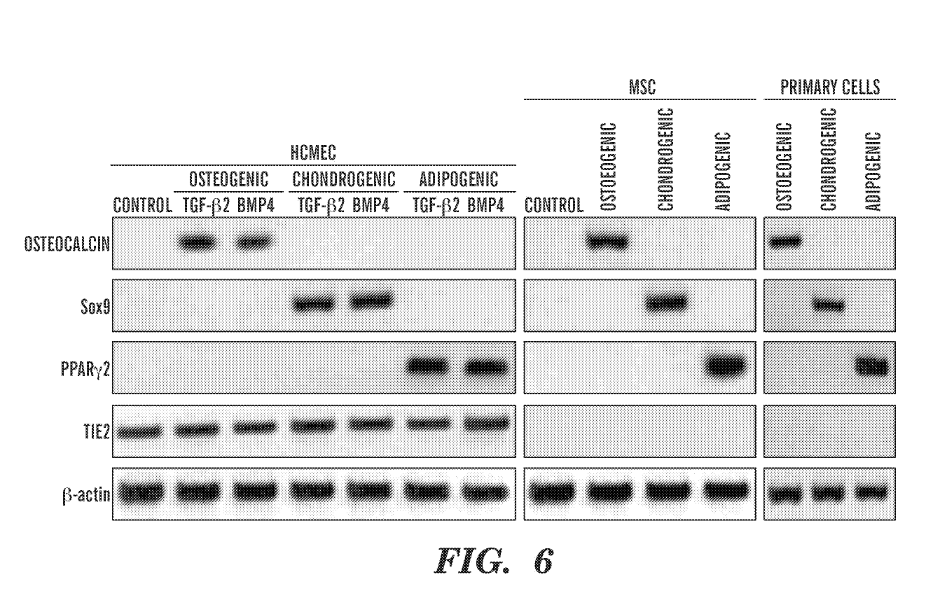

Expression of endothelial markers such as VE-cadherin and CD31 is known to dramatically decrease during EndMT¹. These experiments show that expression of the endothelial-specific protein TIE2 remains constant throughout EndMT (FIG. 1). Therefore, without being bound by theory, it is believed that TIE2 can serve as a marker for endothelial-derived cells that differentiate into other cell types via the endothelial to mesenchymal stem cell mechanism. To confirm that osteoblasts, chondrocytes and adipocytes derived from endothelial cells continue to express TIE2, lysates collected from HCMEC cultures treated with vehicle, TGF-β2 or BMP4 for 48 hours and grown in osteogenic medium for 7 days, chondrogenic medium for 14 days, or adipogenic medium for 7 days, was immunoblotted. Osteogenic medium induced expression of the osteoblastic marker osteocalcin, chondrogenic medium increased expression of the chondrocyte marker SOX9, and adipogenic medium induced expression of the adipocyte marker PPARγ2. Bone marrow-derived mesenchymal stem cells (MSCs) grown in the same differentiation media showed positive expression of these proteins as well, but cells grown in conventional growth medium did not. Most importantly, MSCs and osteoblasts, chondrocytes, and adipocytes derived from MSCs showed no detectable expression of the endothelial marker TIE2. Primary human osteoblasts, chondrocytes, and adipocytes also showed no TIE2 expression. However, osteoblasts, chondrocytes, and adipocytes derived from endothelial cells all showed strong expression of TIE2 (FIG. 6).

Heterotopic ossification can be induced in a mouse model by a constitutively active ALK2 (caALK2) transgene. Constitutively active ALK2 (caALK2) in a transgenic mouse causes heterotopic ossification that is visible using X-ray imagery. Immunohistochemistry was performed for chondrogenic and osteogenic lesions from the caALK2 transgenic mice to investigate expression of Tie2, Sox9, and osteocalcin. Chondrogenic lesions had co-expression of the endothelial marker Tie2 and the chondrocyte marker Sox9. Osteogenic lesions had co-expression of Tie2 and the osteoblast marker osteocalcin. These results suggest that the cells arise from endothelial cells. For comparison, bone and cartilage from the knee joints of wild-type mice showed no evidence of Tie2-positive chondrocytes or osteoblasts (FIG. 9A).

Patients with Fibrodysplasia Ossificans Progressiva (FOP), a disease in which acute inflammation causes heterotopic ossification in soft tissues and formation of an ectopic skeleton²⁰, all carry a heterozygous activating mutation in ALK2²¹⁻²³. To determine if the heterotopic bone and cartilage in these patients could be caused by their differentiation from vascular endothelial cells, immunohistochemistry on lesional tissue from FOP patients using antibodies against TIE2, osteocalcin (for osteoblasts) and SOX9 (for chondrocytes) was performed. Chondrogenic lesions showed co-expression of TIE2 and SOX9, whereas osteogenic lesions showed strong co-expression of the endothelial marker TIE2 and the osteoblast marker osteocalcin in cells lining the calcified tissue. Normal human bone and cartilage from the hip joint showed no evidence of TIE2-positive chondrocytes or osteoblasts (FIG. 9B).

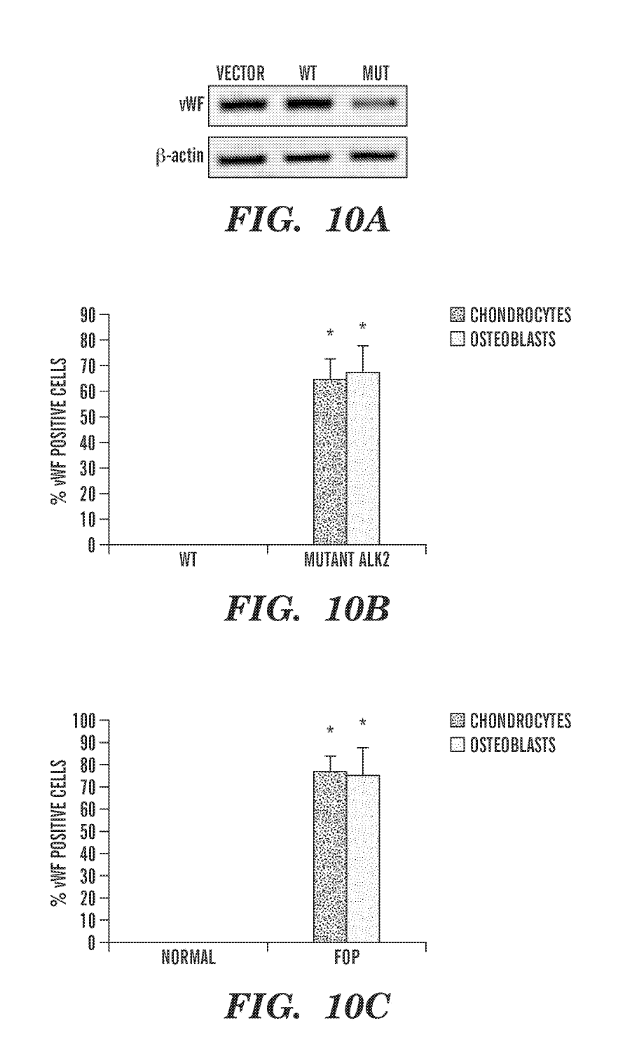

Although expression of most endothelial markers is dramatically reduced during EndMT¹, the existence of other endothelial markers other than TIE2 that would remain expressed in endothelial derived osteoblasts and chondrocytes in FOP lesions was explored. Immunoblotting showed that HCMECs treated with adenoviral mutant ALK2 had reduced levels of von Willebrand Factor (vWF), yet it was still markedly expressed, suggesting that it might be a useful immunohistochemical marker for endothelial cell derived mesenchymal cells (FIG. 10A). Immunostaining for Sox9 and vWF in chondrogenic lesions and osteocalcin and vWF in osteogenic lesions showed positive co-expression in chondrocytes and osteoblasts of heterotopic cartilage and bone of mutant ALK2 transgenic mice. No co-expression was found in bone and cartilage from the knee joints of wild-type mice. (FIG. 10B). Immunohistochemistry was performed to determine expression in the wild type mouse versus mutant ALK2 cells. The analysis showed no evidence of vWF expression in chondrocytes and osteoblasts in cartilage and bone of wild-type mice, but strong co-expression of Sox9 and vWF (chondrocytes) and osteocalcin and vWF (osteoblasts) in heterotopic cartilage and bone in mutant ALK2 transgenic mice. Likewise, heterotopic bone and cartilage from FOP patients contained vWF-positive osteoblasts and chondrocytes, whereas normal human bone and cartilage from the hip joint did not (FIG. 10C). Immunohistochemistry was then performed to determine expression in cells from normal human cartilage and bone versus heterotopic cartilage and bone from FOP patients. No evidence was seen of vWF expression in chondrocytes and osteoblasts of normal human cartilage and bone, but strong co-expression of Sox9 and vWF (chondrocytes) and osteocalcin and vWF (osteoblasts) in heterotopic cartilage and bone from FOP patients.

### Discussion

The findings reported herein provide several novel insights into the mechanism and potential roles of endothelial-mesenchymal transition (EndMT). First, the data demonstrate that EndMT results in generation of mesenchymal stem cells with the potential to differentiate into multiple cell lineages. The current view is that EndMT produces fibroblastic cells that participate in specific developmental processes, in cancer progression and organ fibrosis¹, but the evidence for an endothelial derived stem cell broadens the scope for the role of EndMT in normal development, physiological repair, and disease. For example, the intriguing observation that chondrocytes and osteoblasts at sites of fracture repair are positively stained with antibodies against TIE2²⁸, similar to endothelial derived chondrocytes and osteoblasts at sites of ectopic bone formation in both mice and humans with activating mutations in ALK2, suggests that EndMT may contribute to the physiological process of fracture repair. Given such a possibility, further studies of the sources of chondrocytes and osteoblasts in fracture repair and the related process of distraction osteogenesis²⁹ should be of great interest.

Second, the data indicate that activation/phosphorylation of ALK2 is necessary and sufficient for EndMT to occur in differentiated endothelial cells such as HUVECs and HCMECs under the in vitro conditions used in this study. That the process can occur also in vivo is demonstrated by the finding that a major fraction of the chondrocytes and osteoblasts in ectopic ossifying lesions of mice and humans with an activating mutation in ALK2 are likely to be derived from endothelial cells. These findings, coupled with the in vitro data showing that the process is highly efficient, obviously raises the question of how the process is regulated. The data demonstrate that TGF-β2 and BMP4 are stimulators of EndMT. Furthermore, vascular endothelial growth factor (VEGF) has been found to inhibit EndMT^{30,31}. Therefore, it is believed that VEGF signaling may exert a negative effect on ALK2-mediated EndMT. This may explain why chondrogenesis and not direct (membranous) bone formation is the first step in the ectopic ossification process in FOP patients. It is well established that chondrogenesis only occurs in an anti-angiogenic, hypoxic environment³² and this may well be the condition that favors ALK2 mediated EndMT and chondrogenesis at lesion sites since hypoxia occurs as a result of inflammation³³. In contrast, bone formation requires angiogenesis and osteoblasts produce VEGF³⁴. The factors that cause endothelial cells to convert to chondrocytes in FOP patients are currently unknown. However, it is likely that inflammatory cytokines play a critical role since the ectopic ossifying lesions are triggered by inflammation²⁰.

Third, the data presented here indicate that FOP, in which the hallmark is pathological bone formation, is in fact a vascular disease based on conversion of vascular endothelial cells into multipotent mesenchymal stem cells that subsequently differentiate into cartilage-forming chondrocytes, followed by endochondral ossification. Accumulation of “fibroblastic” cells is an early step in the formation of FOP lesions, and this was previously thought to be a result of fibroblast proliferation²³. The data suggest instead that “fibroblastic” condensation prior to chondrogenesis is the result of EndMT to multipotent stem cells that condense and differentiate into chondrocytes by a process that mimics the early steps in the development of the vertebrate skeleton³². The data show that the majority of the chondrocytes and osteoblasts found in FOP lesions express the endothelial markers TIE2 and vWF. The origin of the small fraction of cells that do not express these markers is unknown, although it is likely that these chondrocytes and osteoblasts arise from mesenchymal stem cells recruited to the lesions from the bone marrow or surrounding tissues.

Finally, given the ease by which vascular endothelial cells can be isolated from umbilical veins, the microvasculature of foreskin, or small skin biopsies of adults, and the efficient cytokine dependent conversion of the cells to mesenchymal stem cells, they may be useful for tissue engineering of skeletal tissues. The data demonstrate that the cells can give rise to chondrocytes, osteoblasts and adipocytes, but given the appropriate culture conditions they may differentiate into other cell types as well.

### Methods

Cell Culture.

Human umbilical vein endothelial cells (HUVEC) and human cutaneous microvascular endothelial cells (HCMEC) were provided by J. Bischoff (Children's Hospital Boston) and isolated as previously described³⁵. Cells were previously tested for purity and found to express no markers for lymphatic endothelial cells or stromal cells (pericytes, smooth muscle cells, etc.)³⁶. Cells were grown in culture using EGM-2 medium (Cambrex), containing 10% FBS and 1% Penicillin/Streptomycin, followed by human endothelial serum free medium (Gibco) 24 hours prior to all experimental conditions. Bone marrow derived mesenchymal stem cells (ScienCell Research Laboratories) were grown in mesenchymal stem cell medium (ScienCell Research Laboratories). Human corneal fibroblasts, from a stock initially provided by E. Hay (Harvard Medical School), were grown in RPMI medium, containing 10% FBS and 1% Penicillin/Streptomycin. Primary human osteoblasts, chondrocytes, and adipocytes and their respective growth media were obtained from Cell Applications Inc. Recombinant TGF-β2 and BMP4 proteins (R&D Systems) were added to the serum free culture medium for all relevant experiments at a concentration of 10 ng/mL. Cells were treated for 15 minutes to assess ALK2 phosphorylation, 1 hour to measure Smad5 phosphorylation, or 48 hours to induce EndMT. All experiments for this study were performed at minimum in triplicate.

Plasmids and Adenoviral Constructs.

Human ACVR1/ALK2 expression constructs were generated by insertion of full-length hACVR1 cDNA (GenBank NW_{—}001105) into the pcDNA 3.1D V5-His-TOPO vector (Invitrogen). The ACVR1/ALK2 R206H mutant construct was generated by site-directed mutagenesis of the normal ACVR1/ALK2 sequence using the Gene Tailor Site-Directed Mutagenesis System (Invitrogen). The oligonucleotides used to generate the mutant construct were: forward 5′-GTACAAAGAACAGTGGCTCaCCAGATTACACTG-3′ (SEQ ID NO: 1); reverse 5′-GTGAGCCACTGTTCTTTGTACCAGAAAAGGAAG-3′ (SEQ ID NO: 2). SpeI and SphI sites were used to generate the adenovirus vectors through the Clontech Adeno-X System (University of Pennsylvania Vector Core). Expression from these constructs was confirmed by sequence analysis and immunoblot analysis. Viral constructs were added to cultures at a concentration of 20 pfu/ml.

Mice.

All procedures were reviewed and approved by the Institutional Animal Care and Use Committee at the Universities of Pennsylvania. Floxed caALK2 transgenic mice³⁷ were a gift from Dr. Yuji Mishina (University of Michigan). To induce expression of caALK2, AV-Cre (Penn Vector Core; 1×10¹¹ particles per mouse) was injected into the left hindlimbs of mice at one month of age. The contralateral limb was injected with empty vector as a control. Heterotopic ossification was detected by X-ray (Senographe DS technology, General Electric Medical Systems, Chalfont St. Giles, UK) at 21 days following AV-Cre injection. Tissues were fixed in isopentane (2-methylbutane) as previously described²³ and cryosections were cut at 10 μm.

Human Tissues.

All FOP patient samples were obtained with informed consent and protocols approved by the Investigational Review Board of the University of Pennsylvania. All biopsies were obtained prior to a diagnosis of FOP since tissue trauma in FOP frequently induces episodes of heterotopic ossification. Normal human bone and cartilage tissue from the hip joint was provided by the Department of Pathology at the Beth Israel Deaconess Medical Center and samples were obtained with informed consent and protocols approved by the Investigational Review Board.

Immunoblotting, Immunoprecipitation, and Immunofluorescence.

Immunoassays were performed using the following antibodies at concentrations (and using protocols) recommended by the respective manufacturers: FSP-1 (H00006275-M01; Stressgen), ALK2 (sc-5671), Phospho-tyrosine (sc-7020), VE-cadherin (sc-6458), TIE2 (sc-324, sc-9026), STRO-1 (sc-47733), CD44 (sc-71220), CD90 (sc-9163), Osteocalcin (sc-74495, sc-23790), SOX9 (sc-20095), COL2A1 (sc-59958, sc-52658), PPARγ2 (sc-22022; Santa Cruz Biotechnology), vWF (ab68545; Abcam); His (A00174; GenScript); α-SMA (A5228), β-actin (A1978; Sigma-Aldrich). Samples were run with Criterion precast SDS-PAGE Gels (Bio-Rad). HRP-conjugated IgG TrueBlot reagents (18-8814, 18-8816, 18-8817; eBioscience) were used at a dilution of 1:1000. TrueBlot IgG beads (eBioscience) were used for immunoprecipitation experiments. Alexa Fluor secondary antibodies (Invitrogen) were used at a dilution of 1:200. Images were acquired using a Nikon 80i fluorescence microscope.

Flow Cytometry.

Endothelial cells were stained in suspension using antibodies against TIE2, FSP-1, STRO-1, and His (described above) and the protocols provided by their respective manufacturer. Flow cytometry was performed at the Harvard Medical School, Department of Pathology flow cytometry core facility using a FACSDCalibur (BD Biosciences) cell sorter isolating 30,000 cells per sample.

Real-Time Quantitative PCR.

RNA extractions were performed using the RNeasy Mini kit (Qiagen) and protocol. RNA samples were submitted to a core facility (Biopolymers Facility, Department of Genetics, Harvard Medical School) where real-time PCR experiments were conducted using the Syber Green PCR system (ABI) on an ABI 7500 cycler, with 40 cycles per sample. Cycling temperatures were as follows: denaturing 95° C.; annealing 60° C.; extension, 70° C. The following primers (Integrated DNA Technologies) were used:

FSP-1:
(SEQ ID NO: 3)
Forward: 5′-TCTTTCTTGGTTTGATCCTG-3′;
(SEQ ID NO: 4)
Reverse: 5′-GCATCAAGCACGTGTCTGAA-3′;
α-SMA:
(SEQ ID NO: 5)
Forward: 5′-GTCCCCATCTATGAGGGCTAT-3′;
(SEQ ID NO: 6)
Reverse: 5′-GCATTTGCGGTGGACAATGGA-3′;
N-cadherin:
(SEQ ID NO: 7)
Forward: 5′-CACCCAACATGTTTACAATCAACAATG-3′;
(SEQ ID NO: 8)
Reverse: 5′-CTGCAGCAACAGTAAGGACAAACATCC-3′;
Fibronectin:
(SEQ ID NO: 9)
Forward: 5′-CCCACCGTCTCAACATGCTTAG-3′;
(SEQ ID NO: 10)
Reverse: 5′-CTCGGCTTCCTCCATAACAAGTAC-3′;
Snail:
(SEQ ID NO: 11 )
Forward: 5′-GCTGCAGGACTCTAATCCAGAGTT-3′;
(SEQ ID NO: 12 )
Reverse: 5′-GACAGAGTCCCAGATGAGCATTG-3′;
Slug:
(SEQ ID NO: 13)
Forward: 5′-TGTTGCAGTGAGGGCAAGAA-3′;
(SEQ ID NO: 14)
Reverse: 5′-GACCCTGGTTGCTTCAAGGA-3′;
ZEB-1:
(SEQ ID NO: 15 )
Forward: 5′-TTCAAACCCATAGTGGTTGCT-3′;
(SEQ ID NO: 16)
Reverse: 5′-TGGGAGATACCAAACCAACTG-3′;
SIP-1:
(SEQ ID NO: 17)
Forward: 5′-CAAGAGGCGCAAACAAGC-3′;
(SEQ ID NO: 18)
Reverse: 5′-GGTTGGCAATACCGTCATCC-3′;
LEF-1:
(SEQ ID NO: 19)
Forward: 5′-CCGAAGAGGAAGGCGATTTAGC-3′;
(SEQ ID NO: 20)
Reverse: 5'-GGTCCCTTGTTGTAGAGGCC-3';
Twist:
(SEQ ID NO: 21)
Forward: 5′-GGAGTCCGCAGTCTTACGAG-3′;
(SEQ ID NO: 22)
Reverse: 5′-TCTGGAGGACCTGGTAGAGG-3′;
CD10:
(SEQ ID NO: 23)
Forward: 5′-AACATGGATGCCACCACTGAG-3′;
(SEQ ID NO: 24)
Reverse: 5′-CACATATGCTGTACAAGCCTC-3′;
CD13:
(SEQ ID NO: 25)
Forward: 5′-AAGCTCAACTACACCCTCAGC-3′;
(SEQ ID NO: 26)
Reverse: 5′-GGGTGTGTCATAATGACCAGC-3′;
CD44:
(SEQ ID NO: 27)
Forward: 5′-CGGACACCATGGACAAGTTT-3′;
(SEQ ID NO: 28)
Reverse: 5′-GAAAGCCTTGCAGAGGTCAG-3′;
CD73:
(SEQ ID NO: 29)
Forward: 5′-AAGGAAGGGGAAGAACAGGA-3′;
(SEQ ID NO: 30)
Reverse: 5′-GGCAGAGCTGATGGAATCTC-3′;
CD90:
(SEQ ID NO: 31)
Forward: 5′-CAGGTGATCCGTCCGGCAA-3′;
(SEQ ID NO: 32)
Reverse: 5′-GAGAGGCGTGGAGACC-3′;
CD120a:
(SEQ ID NO: 33)
Forward: 5′-CCTACTTGTACAATGACTGTC-3′;
(SEQ ID NO: 34)
Reverse: 5′-TGCATGGCAGGTGCACACG-3′;
CD124:
(SEQ ID NO: 35)
Forward: 5′-AAATCGTGAACTTTGTCTCCGT-3′;
(SEQ ID NO: 36)
Reverse: 5′-CCCAGTGCCCTCTACTCTCAT-3′;
Osteocalcin:
(SEQ ID NO: 37)
Forward: 5′-AGGCGCTACCTGTATCAATGG-3′;
(SEQ ID NO: 38)
Reverse: 5′-TAGACCGGGCCGTAGAAGC-3′;
Osterix:
(SEQ ID NO: 39)
Forward: 5′-AACCCCCAGCTGCCCACCTACC-3′;
(SEQ ID NO: 40)
Reverse: 5′-GACGCTCCAGCTCATCCGAACG-3′;
COL2A1:
(SEQ ID NO: 41)
Forward: 5′-TTCAGCTATGGAGATGACAATC-3′;
(SEQ ID NO: 42)
Reverse: 5′-AGAGTCCTAGAGTGACTGAG-3′;
SOX9:
(SEQ ID NO: 43)
Forward: 5′-AGACCTTTGGGCTGCCTTAT-3′;
(SEQ ID NO: 44)
Reverse: 5′-TAGCCTCCCTCACTCCAAGA-3′;
Adiponectin:
(SEQ ID NO: 45)
Forward: 5′-CATGACCAGGAAACCACGACT-3′;
(SEQ ID NO: 46 )
Reverse: 5'-TGAATGCTGAGCGGTAT-3';
PPARγ2:
(SEQ ID NO: 47)
Forward: 5′-AGGAGCAGAGCAAAGAGGTG-3′;
(SEQ ID NO: 48)
Reverse: 5′-AGGACTCAGGGTGGTTCAGC-3′;
GAPDH:
(SEQ ID NO: 49)
Forward: 5′-GAAGGTGAAGGTCGGAGTC-3′;
(SEQ ID NO: 50)
Reverse: 5′-GAAGATGGTGATGGGATTTC-3′.

Cell Differentiation.

Cells were grown in StemPro osteogenic, chondrogenic, or adipogenic culture medium (Invitrogen). Alkaline phosphatase staining was performed using the alkaline phosphatase kit and protocol (Sigma-Aldrich) on cultures grown in osteogenic medium for 7 days to detect osteoblasts. Alizarin Red (Sigma-Aldrich) staining was performed for 30 minutes on cultures grown in osteogenic medium for 21 days to detect matrix calcification. Alcian Blue (Sigma) staining was used to stain chondrocyte proteoglycans for 5 minutes in cultures grown in chondrogenic medium for 14 days. Oil Red O (Sigma-Aldrich) staining was performed for 15 minutes on cultures grown in adipogenic medium for 7 days.

RNA Interference.

siRNA gene expression knockdown studies were performed using the TriFECTa RNAi kit (Integrated DNA Technologies) and corresponding protocol. Each 27 mer siRNA duplex was transfected into cells using X-tremeGene siRNA transfection reagent (Roche) following the manufacturer's guidelines. siRNA was synthesized (Integrated DNA Technologies) with the following sequences: ALK2: 5′-GCAACACUGUCCAUUCUUCUUAACCAG-3′ (SEQ ID NO: 51); negative control: 5′-UCACAAGGGAGAGAAAGAGAGGAAGGA-3′ (SEQ ID NO: 52).

Statistical Analyses.

One-way analysis of variance (ANOVA) was performed and confirmed with two-tailed paired student's t test using GraphPad Prism 4 software. P values less than 0.05 were considered significant.

### REFERENCES

### Background, Detailed Description and Example 1

1. Potenta, S., Zeisberg, E. & Kalluri, R. The role of endothelial-to-mesenchymal transition in cancer progression. Br. J. Cancer 99, 1375-1379 (2008).
2. Akhurst, R. J. & Derynck, R. TGF-beta signaling in cancer—a double-edged sword. Trends Cell Biol. 11, S44-S51 (2001).
3. Thiery, J. P. Epithelial-mesenchymal transitions in tumor progression. Nat. Rev. Cancer 2, 442-454 (2002).
4. Thiery, J. P. Epithelial-mesenchymal transitions in development and pathologies. Curr. Opin. Cell Biol. 15, 740-746 (2003).
5. Hay, E. D. The mesenchymal cell, its role in the embryo, and the remarkable signaling mechanisms that create it. Dev. Dyn. 233, 706-720 (2005).
6. Boyer, A. S. et al. TGFbeta2 and TGFbeta3 have separate and sequential activities during epithelial-mesenchymal cell transformation in the embryonic heart. Dev. Biol. 208, 530-545 (1999).
7. Lai, Y. T. et al. Activin receptor-like kinase 2 can mediate atrioventricular cushion transformation. Dev. Biol. 222, 1-11 (2000).
8. Camenisch, T. D. et al. Temporal and distinct TGFbeta ligand requirements during mouse and avian endocardial cushion morphogenesis. Dev. Biol. 248, 170-181 (2002).
9. Wang, J. et al. Atrioventricular cushion transformation is mediated by ALK2 in the developing mouse heart. Dev. Biol. 286, 299-310 (2005).
10. Okagawa, H., Markwald, Y. & Sugi, Y. Functional BMP receptor in endocardial cells is required in atrioventricular cushion mesenchymal cell formation in the chick. Dev. Biol. 306, 179-192 (2007).
11. Azhar, M. et al. Ligand-specific function of transforming growth factor beta in epithelial-mesenchymal transition in heart development. Dev Dyn. 238, 431-442 (2009).
12. Zeisberg, E. M., Potenta, S., Xie, L., Zeisberg, M. & Kalluri, R. Discovery of endothelial to mesenchymal transition as a source for carcinoma-associated fibroblasts. Cancer Res. 67, 10123-10128 (2007).
13. Zeisberg, E. M. et al. Endothelial-to-mesenchymal transition contributes to cardiac fibrosis. Nat. Med. 13, 952-961 (2007).
14. Zeisberg, E. M., Potenta, S. E., Sugimoto, H., Zeisberg, M. & Kalluri, R. Fibroblasts in kidney fibrosis emerge via endothelial-to-mesenchymal transition. J. Am. Soc. Nephrol. 19, 2282-2287 (2008).
15. Kizu, A., Medici, D. & Kalluri, R. Endothelial-mesenchymal transition as a novel mechanism for generating myofibroblasts during diabetic nephropathy. Am. J. Pathol. 175, 1371-1373 (2009).
16. Li, J., Qu, X. & Bertram, J. F. Endothelial-myofibroblast transition contributes to the early development of diabetic renal interstitial fibrosis in streptozotocin-induced diabetic mice. Am. J. Pathol. 175, 1380-1388 (2009).
17. Mironov, V. et al. Endothelial-mesenchymal transformation in atherosclerosis: a recapitulation of embryonic heart tissue morphogenesis. Ann. Biomed. Eng. 23, S29A (1995).
18. Arciniegas, E., Frid, M. G., Douglas, I. S. & Stenmark, K. R. Perspectives on endothelial-to-mesenchymal transition: potential contribution to vascular remodeling in chronic pulmonary hypertension. Am. J. Physiol. Lung Cell Mol. Physiol. 293, L1-8 (2007).
19. Lee, J. G. & Kay, E. P. FGF-2-mediated signal transduction during endothelial mesenchymal transformation in corneal endothelial cells. Exp. Eye Res. 83, 1309-1316 (2006).
20. Shore, E. M. & Kaplan, F. S. Insights from a rare genetic disorder of extra-skeletal bone formation, fibrodysplasia ossificans progressiva (FOP). Bone 43, 427-433 (2008).
21. Kaplan, F. S. et al. Skeletal metamorphosis in fibrodysplasia ossificans progressiva (FOP). J. Bone Miner. Metab. 26, 521-530 (2008).
22. Shore, E. M. et al. A recurrent mutation in the BMP type I receptor ACVR1 causes inherited and sporadic fibrodysplasia ossificans progressiva. Nat. Genet. 38, 525-527 (2006).
23. Lounev, V. Y. et al. Identification of progenitor cells that contribute to heterotopic skeletogenesis. J. Bone Joint Surg. Am. 91, 652-663 (2009).
24. Kalluri, R. & Weinberg, R. A. The basics of epithelial-mesenchymal transition. J. Clin. Invest. 119, 1420-1428 (2009).
25. Chen, D., Zhao, M. & Mundy, G. R. Bone morphogenetic proteins. Growth Factors 22, 233-241 (2004).
26. Mercado-Pimentel, M. E. & Runyan R. B. Multiple transforming growth factor-beta isoforms and receptors function during epithelial-mesenchymal cell transformation in the embryonic heart. Cells Tissues Organs 185, 146-156 (2007).
27. Chamberlain, G., Fox, J., Ashton, B. & Middleton, J. Concise review: mesenchymal stem cells: their phenotype, differentiation capacity, immunological features, and potential for homing. Stem Cells 25, 2739-2749 (2007).
28. Lewinson, D. et al. Expression of vascular antigens by bone cells during bone regeneration in a membranous bone distraction system. Histochem. Cell Biol. 116, 381-388 (2001).
29. Ilizarov, G. A. The principles of the Ilizarov method. Bull. Hosp. Jt. Dis. Orthop. Inst. 48, 1-11 (1988).
30. Paruchuri, S. et al. Human pulmonary valve progenitor cells exhibit endothelial/mesenchymal plasticity in response to vascular endothelial growth factor-A and transforming growth factor-beta2. Circ. Res. 99, 861-869 (2006).
31. Goumans, M. J., van Zonneveld, A. J. & ten Dijke, P. Transforming growth factor beta-induced endothelial-to-mesenchymal transition: a switch to cardiac fibrosis? Trends Cardiovasc. Med. 18, 293-298 (2008).
32. Amarilio, R. et al. HIF1alpha regulation of Sox9 is necessary to maintain differentiation of hypoxic prechondrogenic cells during early skeletogenesis. Development 134, 3917-3928 (2007).
33. Karhausen, J., Haase, V. H. & Colgan, S. P. Inflammatory hypoxia: role of hypoxia-inducible factor. Cell Cycle 4, 256-258 (2005).
34. Dai, J. & Rabie, A. B. VEGF: an essential mediator of both angiogenesis and endochondral ossification. J. Dent. Res. 86, 937-950 (2007).
35. Boye, E. et al. Clonality and altered behavior of endothelial cells from hemangiomas. J. Clin. Invest. 107, 745-752 (2001).
36. Jinnin, M. et al. Suppressed NFAT-dependent VEGFR1 expression and constitutive VEGFR2 signaling in infantile hemangioma. Nat. Med. 14, 1236-1246 (2008).
37. Fukuda, T. et al. Generation of a mouse with conditionally activated signaling through the BMP receptor, ALK2. Genesis 44, 159-167 (2006).

### Example 2

The experiments detailed herein further show that chondrocytes and osteoblasts from FOP lesions express endothelial markers suggesting that they are derived from vascular endothelial cells. Lineage tracing of heterotopic cartilage and bone also indicates an endothelial origin. Expressing the mutant (R206H) ALK2 in human endothelial cells causes EndMT and acquisition of a multipotent stem cell-like phenotype. EndMT is promoted by treating cells with TGF-β2 or BMP4, and this is prevented by inhibitory ALK2-specific siRNA.

### Endothelial Origin of Heterotopic Cartilage and Bone

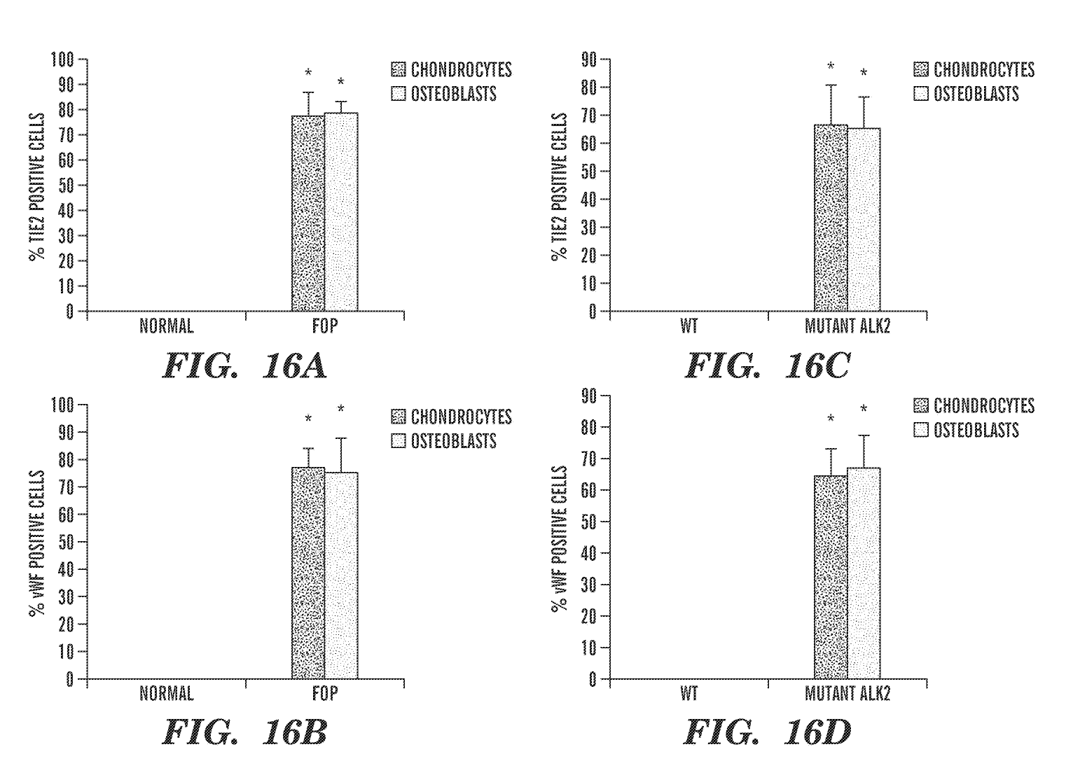

To determine whether formation of heterotopic bone and cartilage in FOP patients could be caused by EndMT, immunohistochemistry was performed on lesional tissue from FOP patients using antibodies specific for the endothelial markers TIE2 and von Willebrand Factor (vWF) and specific for osteocalcin and SOX9 to detect osteoblasts and chondrocytes, respectively. Cells in chondrogenic lesions showed co-expression of TIE2 and vWF with SOX9, whereas osteogenic lesions showed strong co-expression of TIE2 and vWF with osteocalcin in cells lining the calcified tissue. Normal human bone and cartilage from the hip joint showed no evidence of TIE2 or vWF positive chondrocytes or osteoblasts (FIG. 16A, B). In a mouse model of heterotopic ossification induced by a constitutively active ALK2 (caALK2) transgene, immunohistochemistry of chondrogenic and osteogenic lesions showed Tie2 and vWF positive chondrocytes and osteoblasts as indicated by co-staining for Tie2 and vWF with Sox9 or Tie2 and vWF with osteocalcin, respectively, suggesting that they arise from endothelial cells. For comparison, bone and cartilage from the knee joints of wild-type mice showed no evidence of Tie2 or vWF positive chondrocytes or osteoblasts (FIG. 16C, D).

To confirm the endothelial origin of heterotopic cartilage and bone, IRG reporter mice crossed with mice carrying a Tie2-Cre transgene were used for cell lineage tracing by immunohistochemistry, performed on BMP4-induced heterotopic cartilage and bone in Tie2-Cre reporter mice. In the offspring, enhanced green fluorescent protein (EGFP) is expressed in cells of Tie2-positive lineage. Staining of the heterotopic cartilage and bone, induced by injecting the ALK2 activating ligand BMP4 intramuscularly, with antibodies specific for Sox9 and osteocalcin, demonstrated that most of the chondrocytes and osteoblasts were EGFP positive. Furthermore, these EGFP positive cells showed co-expression with the endothelial markers vWF, Tie1, and VE-cadherin

### Mutation in ALK2 Causes Endothelial-Mesenchymal Transition

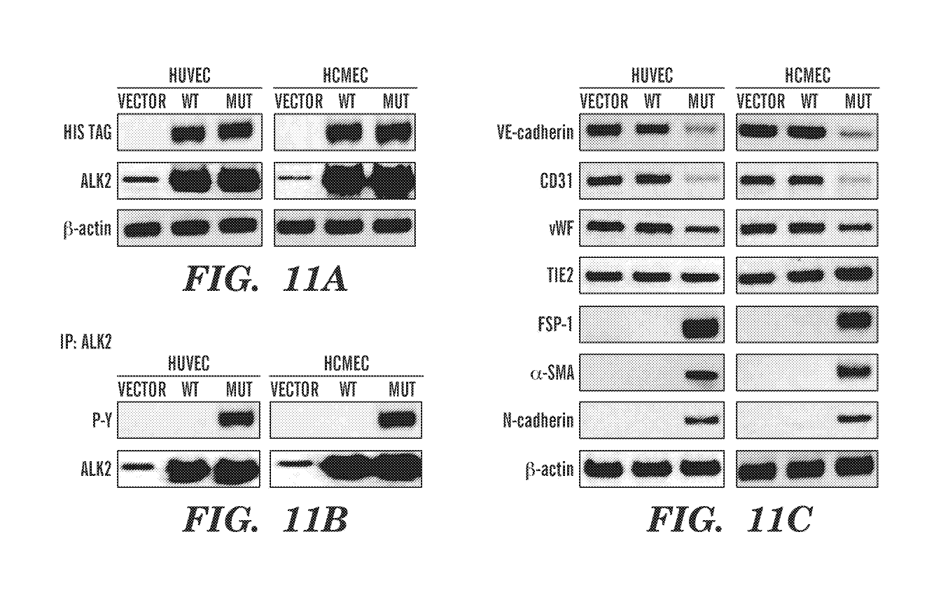

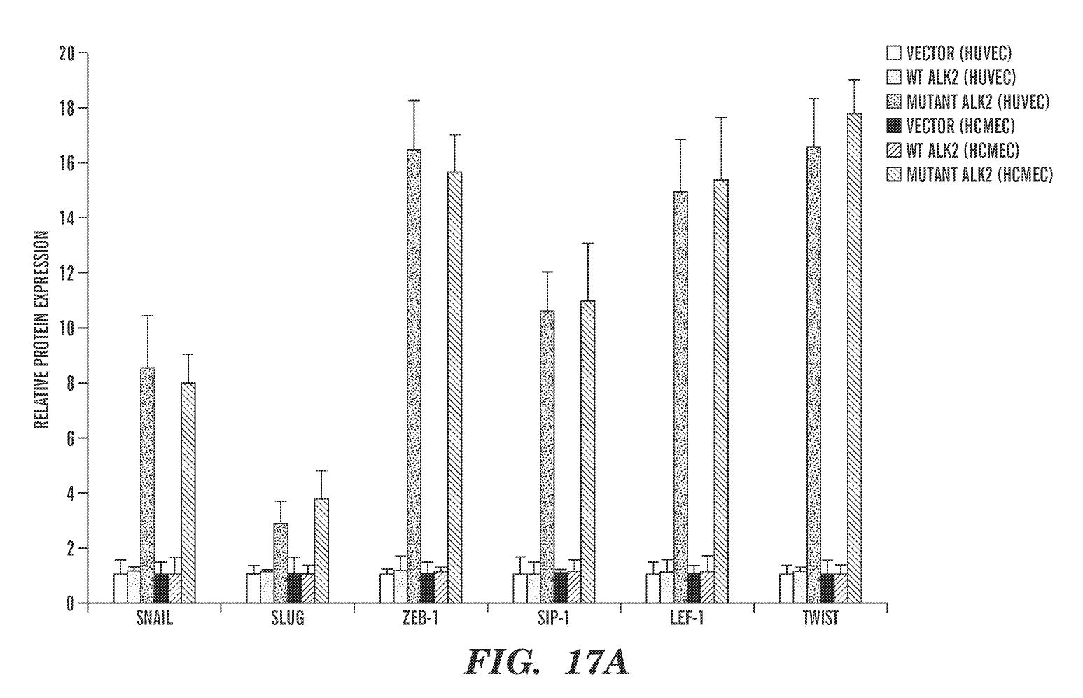

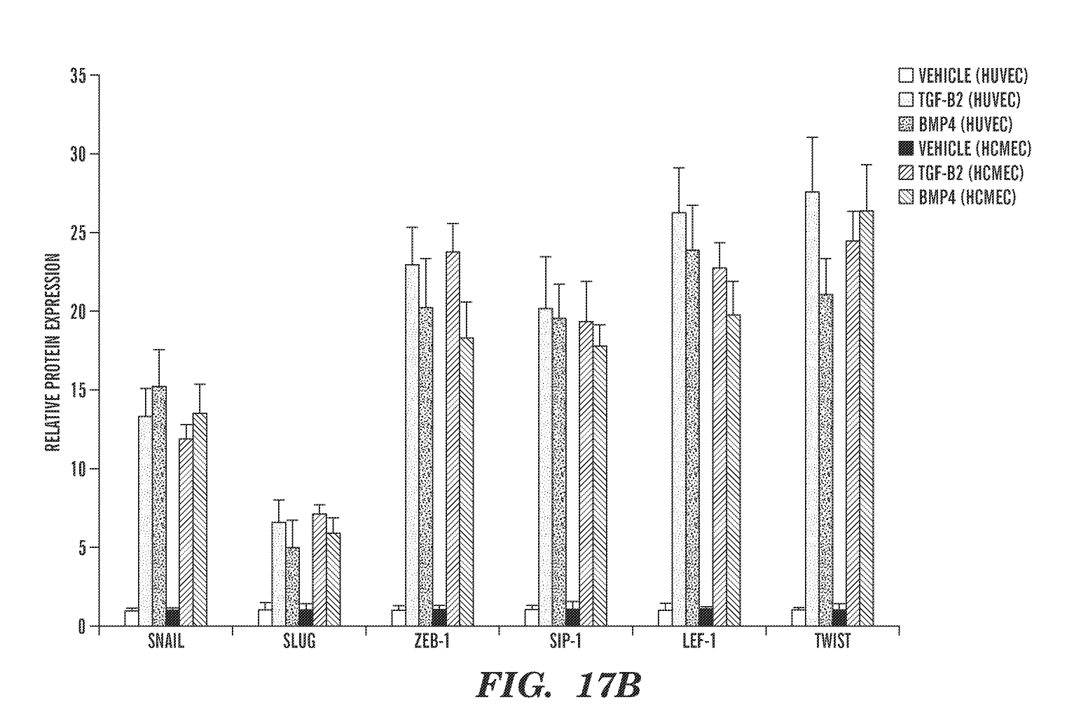

Infecting human umbilical vein endothelial cells (HUVECs) and human cutaneous microvascular endothelial cells (HCMECs) with adenoviral constructs encoding wild-type or mutant (R206H) ALK2 resulted in greatly increased levels of ALK2 (FIG. 11A). Immunoblotting also showed that the mutation induced phosphorylation of ALK2, whereas infection with constructs encoding wild-type ALK2 or vector did not (FIG. 11B). Mutant ALK2 expression caused a change in endothelial cell shape to a mesenchymal morphology and induced co-expression of the mesenchymal marker FSP-1 and the endothelial marker TIE2, as determined by DIC imaging and flow cytometry. Flow cytometry analysis was used to determine co-expression of TIE2 and FSP-1 in cells containing the mutant ALK2 construct. EndMT was confirmed by immunoblotting showing that expression of mutant ALK2 reduced levels of endothelial markers VE-cadherin, CD31, and vWF and increased expression of mesenchymal markers FSP-1, α-SMA, and N-cadherin. TIE2 expression did not change (FIG. 11C). Increased expression of EndMT-associated transcription factors (24) Snail, Slug, ZEB-1, SIP-1, LEF-1, and Twist in cells expressing mutant ALK2 was confirmed by ELISA (FIG. 17A).

Heterotopic ossification begins with mesenchymal condensation prior to chondrogenesis (20). Immunohistochemistry was performed on early stage BMP4-induced lesions of heterotopic ossification in Tie2-Cre reporter mice. The Tie2-Cre reporter mice showed EGFP positive mesenchymal cells (co-stained with FSP-1 antibodies) in early lesions of BMP4-induced heterotopic ossification. Most of the EGFP positive cells also expressed endothelial markers vWF, Tie1, and VE-cadherin.

### EndMT Induces a Stem Cell-Like Phenotype

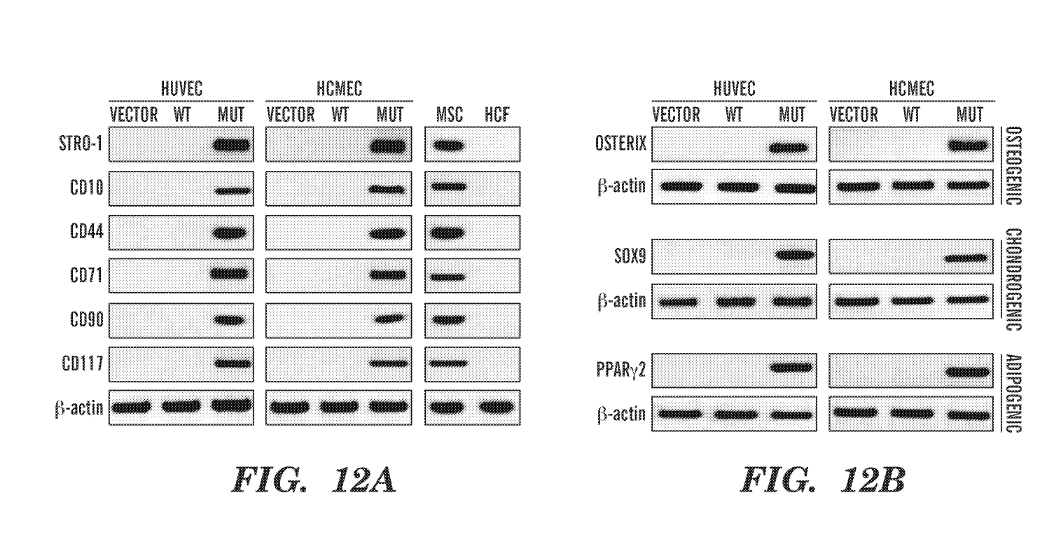

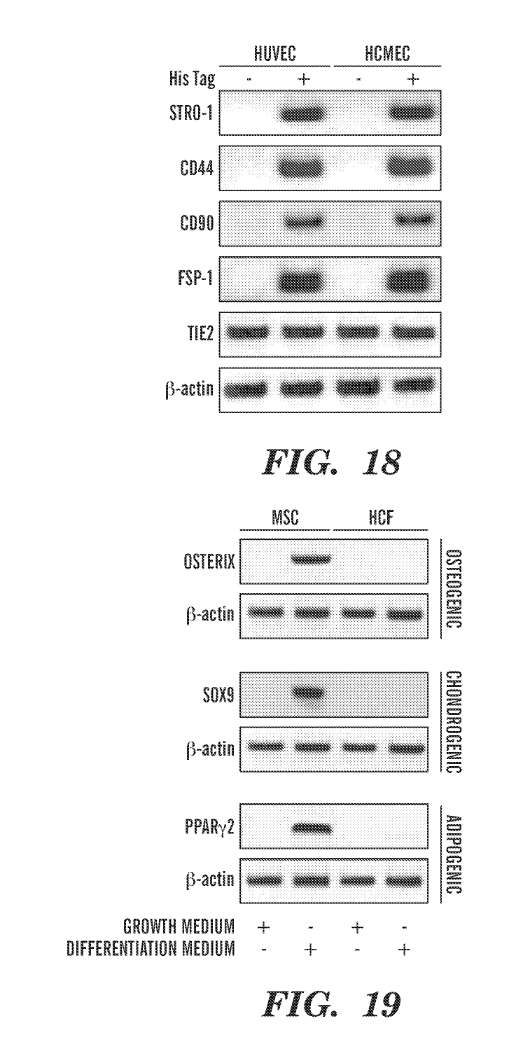

To find out whether endothelial cells expressing mutant ALK2 acquire a stem cell-like phenotype, flow cytometry of cells was performed with antibodies specific for the endothelial marker TIE2 and the mesenchymal stem cell marker STRO-1. Mutant ALK2 expressing cells showed co-expression of the two proteins but no STRO-1 expression was found in wild-type ALK2 or vector treated cells. Immunoblotting showed that lysates of cells treated with adenoviral mutant ALK2 expressed the stem cell markers (25) STRO-1, CD10, CD44, CD71, CD90 and CD117. Lysates of bone marrow-derived mesenchymal stem cells also showed positive expression of all these proteins, but adult human corneal fibroblasts did not (FIG. 12A). In addition, when endothelial cells treated with adenoviral mutant ALK2 were stained with antibodies specific for the His-tagged protein and separated by fluorescence activated cell sorting, only the His-tag positive cell population expressed mesenchymal (FSP-1) and stem cell (STRO-1, CD44, CD90) markers by immunoblotting (FIG. 18).

Since mesenchymal stem cells are multipotent, the differentiation capabilities of endothelial cells that undergo EndMT were also assessed. Endothelial cells were exposed to adenoviral expression constructs for 48 h, and then grown in osteogenic, chondrogenic, or adipogenic media. Immunoblotting using antibodies specific for osteoblast (osterix), chondrocyte (SOX9), and adipocyte (PPARγ2) markers showed that mutant ALK2 increased expression of these markers when cells were grown in their respective differentiation medium (FIG. 12B). Cultures treated with mutant ALK2 showed positive staining for alkaline phosphatase 7 d after osteogenic medium was added, whereas wild-type ALK2 or vector treated cultures showed none. Cells treated with mutant ALK2 and grown in osteogenic medium for 21 d showed high levels of matrix calcification by alizarin red staining. Vector treated cells showed no alizarin red staining. Similar results were found for cells grown in chondrogenic medium for 14 d using the cartilage proteoglycan stain alcian blue, or in adipogenic medium for 7 d using oil red O staining. The differentiation potential of endothelial derived mesenchymal stem-like cells was similar to that of bone marrow derived mesenchymal stem cells. Human corneal fibroblasts did not exhibit differentiation activity under the same conditions (FIG. 19). Differentiation was also seen to occur when polylactic acid sponges, containing HUVECs or HCMECs pretreated with the adenoviral constructs, were implanted into immunodeficient nude mice, followed by local injection of osteogenic, chondrogenic, or adipogenic differentiation medium every 72 h for 6 weeks. The products in the polylactic acid scaffolds containing the transformed endothelial cells were seen to stain positive for osteoblasts (alizarin red), chondrocyte (alcian blue), or adipocyte (oil red O), respectively.

### Ligand-Specific Induction of EndMT

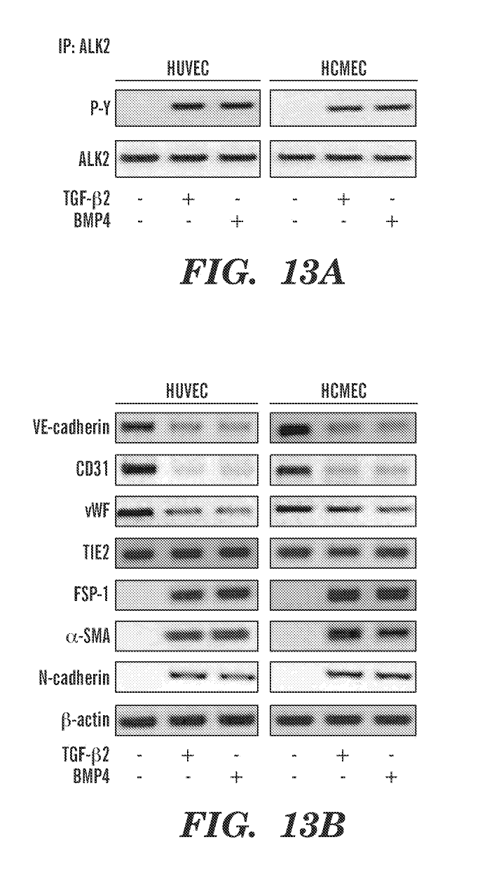

TGF-β2 and BMP4 are ligands known to activate ALK226,27. Receptor phosphorylation was therefore examined in endothelial cells treated with these factors. Immunoprecipitation assays using antibodies specific for ALK2, followed by immunoblotting with phospho-tyrosine (P-Y) specific antibodies indicated that treatment with TGF-β2 or BMP4 for 15 min induced receptor phosphorylation, but treatment with vehicle did not (FIG. 13A). HUVECs and HCMECs treated with recombinant TGF-β2 or BMP4 for 48 h showed a distinct change from cobblestone-like endothelial cell morphology to fibroblast-like morphology. When cells were co-stained using antibodies specific for the endothelial marker TIE2 and the mesenchymal marker FSP-1 and analyzed by flow cytometry, vehicle treated cells were positive for TIE2, but showed no staining for FSP-1. TGF-β2 or BMP4 treated cells showed staining for both. DIC imaging indicated a change in cell morphology. Immunoblotting showed that VE-cadherin, CD31, and vWF levels were decreased in cells treated with TGF-β2 or BMP4, while FSP-1, α-SMA, and N-cadherin levels increased. TIE-2 expression levels remained unchanged (FIG. 13B). ELISA analysis of EndMT-associated transcription factors Snail, Slug, ZEB-1, SIP-1, LEF-1, and Twist, revealed that expression of these proteins was much higher in cells treated with TGF-β2 or BMP4 than in control cells (FIG. 17B).

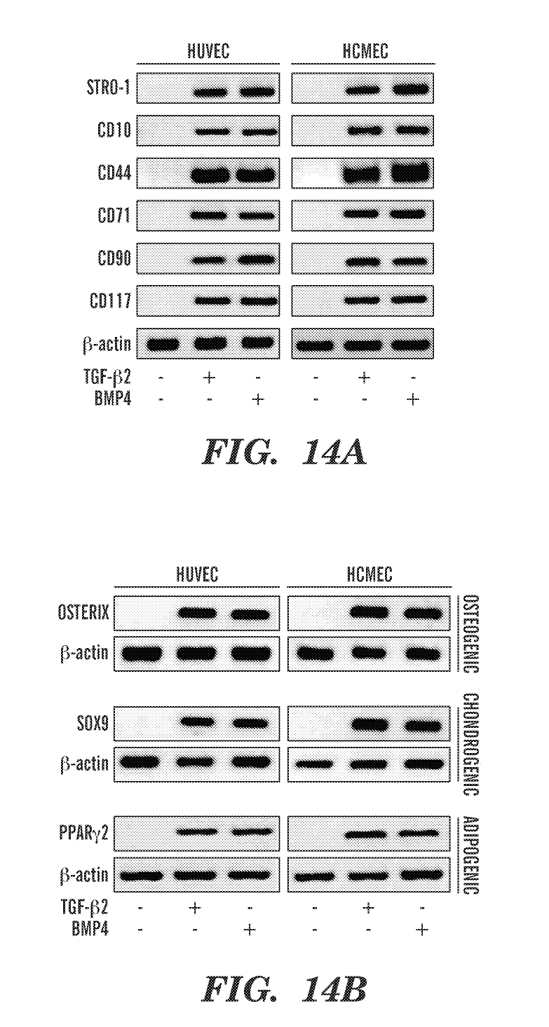

To further assess acquisition of a stem cell-like phenotype, endothelial cells were co-stained with antibodies specific for the endothelial marker TIE2 and the mesenchymal stem cell marker STRO-1. Cells treated with TGF-β2 or BMP4 showed massive co-expression of both markers, but vehicle treated cells showed no expression of STRO-1 as analyzed by flow cytometry. Immunoblotting of lysates collected from cells treated under the same experimental conditions showed that STRO-1 and other mesenchymal stem cell markers CD10, CD44, CD71, CD90, and CD117 were not expressed in vehicle treated cells, but were strongly expressed in cells treated with TGF-β2 or BMP4 (FIG. 14A).

To assess differentiation potential, endothelial cells were treated with vehicle, TGF-β2, or BMP4 for 48 h, followed by growth in osteogenic, chondrogenic or adipogenic culture medium. Immunoblotting using lysates from endothelial cells treated with TGF-β2 or BMP4 and then grown in osteogenic medium for 7 d, chondrogenic medium for 14 d or adipogenic medium for 7 d demonstrated increases in protein expression of the osteoblast marker osterix, the chondrocyte marker SOX9, and the adipocyte marker PPARγ2. Vehicle treated cells did not express these markers (FIG. 14B). Cultures treated with TGF-β2 or BMP4 and stained for alkaline phosphatase after 7 d in osteogenic medium, with alizarin red after 21 d in osteogenic medium, alcian blue after 14 d in chondrogenic medium, or oil red O after 7 d in adipogenic medium, differentiated into other cell types, whereas those treated with vehicle did not.

Multipotency was confirmed in vivo by implanting polylactic acid sponges containing endothelial cells pre-treated with vehicle, TGF-β2, or BMP4 into immunodeficient nude mice, followed by local injection of osteogenic, chondrogenic, or adipogenic differentiation medium every 72 h for 6 weeks. Only implants with cells treated with TGF-β2 or BMP4 showed formation of bone, cartilage, or fat products in the polylactic acid scaffolds as determined by staining of osteoblast (alizarin red), chondrocyte (alcian blue), or adipocyte (oil red O) products. In further experiments, HUVECs and HCMECs were pre-labeled with fluorescent quantum dots prior to treatment and implantation. Endothelial derived stem-like cell differentiation was tracked in vivo by immunohistochemistry of sections of polylactic acid scaffolds containing endothelial cells pre-labeled with fluorescent quantum dots (Qtracker). Scaffolds treated with vehicle, TGF-β2, or BMP4 were analyzed. The data demonstrate co-expression of the Qtracker with osteocalcin, SOX9, or adiponectin in implants of cells treated with TGF-β2 or BMP4, but not with vehicle, and injected with osteogenic, chondrogenic, or adipogenic medium, respectively.

### Receptor Specificity in EndMT

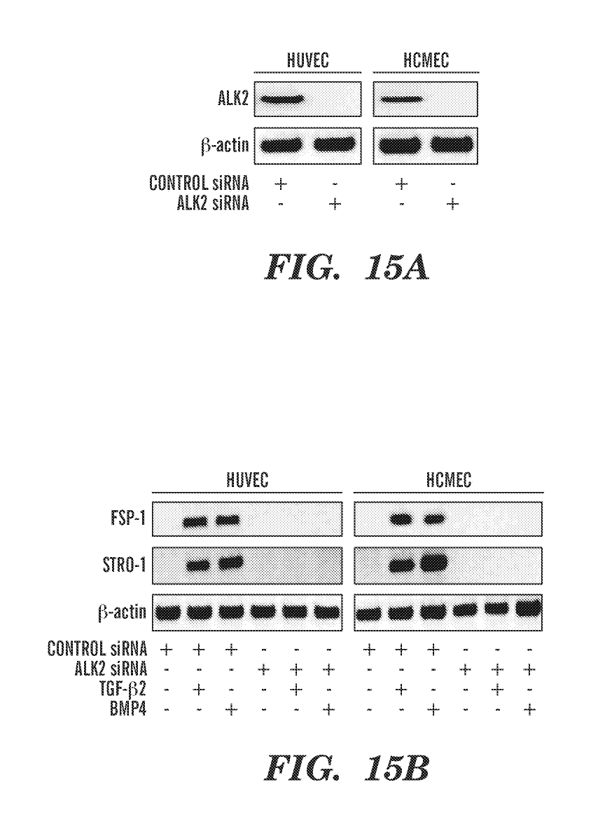

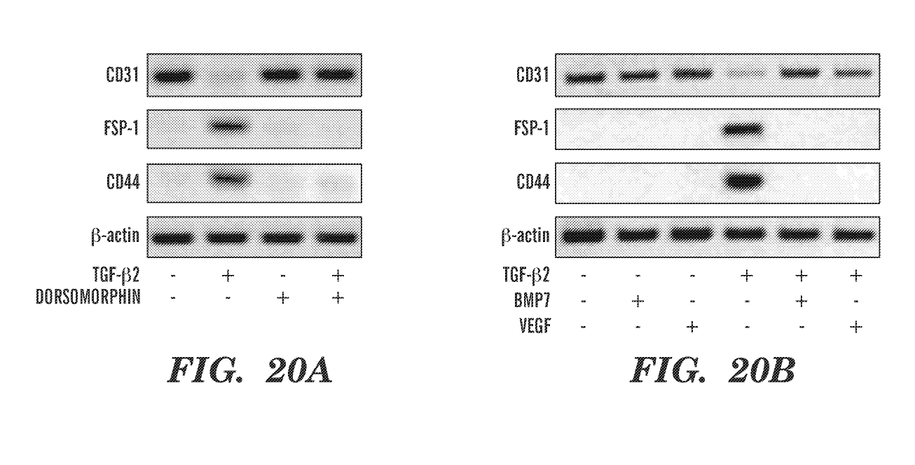

Whether ALK2 is necessary for acquisition of the multipotent stem cell-like phenotype in endothelial cells was then investigated. Cells were transfected for 24 h with a siRNA duplex specific for knockdown of ALK2. A scrambled non-specific siRNA duplex was used as a negative control. Immunoblotting of cell lysates showed complete inhibition of ALK2 expression by the ALK2-specific siRNA (FIG. 15A). Transfected cells were subsequently treated with vehicle, TGF-β2, or BMP4 for 48 h, followed by lysis and immunoblotting for the mesenchymal and stem cell markers FSP-1 and STRO-1. TGF-β2 or BMP4 treated cells transfected with control siRNA had much higher expression of FSP-1 and STRO-1 than vehicle-treated cells, whereas ALK2 siRNA treatment blocked these increases (FIG. 15B). These results were confirmed by flow cytometry of cells stained in suspension with antibodies specific for TIE2 and FSP-1. Vehicle treated cells transfected with control siRNA or ALK2 siRNA showed no positive staining for FSP-1. TGF-β2 or BMP4 treated cells transfected with control siRNA showed most cells expressing FSP-1, while cultures transfected with ALK2 siRNA showed very few cells expressing FSP-1. Treatment with the ALK2 inhibitor dorsomorphin also blocked EndMT (FIG. 20A).

When cells grown under the same experimental conditions were cultured in osteogenic medium for 7 d, TGF-β2 or BMP4 treated cultures that were transfected with control siRNA showed positive staining for alkaline phosphatase, whereas vehicle treated cultures and cells transfected with ALK2 siRNA showed none. Similar results were found for alizarin red staining after 21 d of incubation in osteogenic medium, chondrocyte proteoglycans by alcian blue staining after 14 d of incubation in chondrogenic medium, and adipocytes by oil red O staining after 7 d of incubation in adipogenic medium.

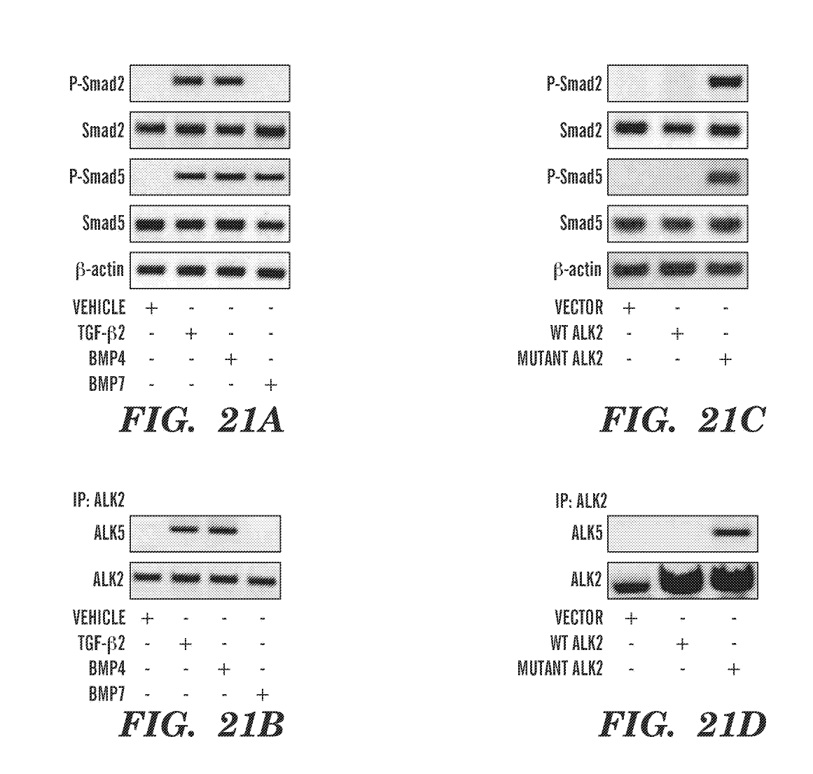

Interestingly, while some ALK2 ligands (TGF-β2, BMP4) stimulate EndMT, BMP7 is an ALK2 activating ligand that inhibits EndMT13 (FIG. 20B). To determine the differences in signaling induced by different ALK2 ligands, phosphorylation levels of Smad2 and Smad5 were assessed. Immunoblotting showed that 1-h of treatment with TGF-β2 or BMP4 promoted phosphorylation of both Smad2 and Smad5, whereas BMP7 stimulated phosphorylation of only Smad5 (FIG. 21A). Since ALK2 is commonly associated with Smad5, but not Smad226, potential interaction of ALK2 with ALK5, which is known to induce phosphorylation of Smad22 was investigated. Immunoblotting of lysates immunoprecipitated with ALK2 antibodies showed the presence of ALK5 in precipitates from cells treated with TGF-β2 or BMP4. No interaction of ALK2 and ALK5 was found in vehicle or BMP7 treated cells (FIG. 21B). This signaling specificity was further confirmed in the case of the mutant (R206H) ALK2. Immunoprecipitation and immunoblotting of lysates from endothelial cells expressing mutant ALK2 showed interaction of ALK2 with ALK5 and phosphorylation of both Smad2 and Smad5, whereas cells treated with wild-type ALK2 or vector did not (FIG. 21C, D).

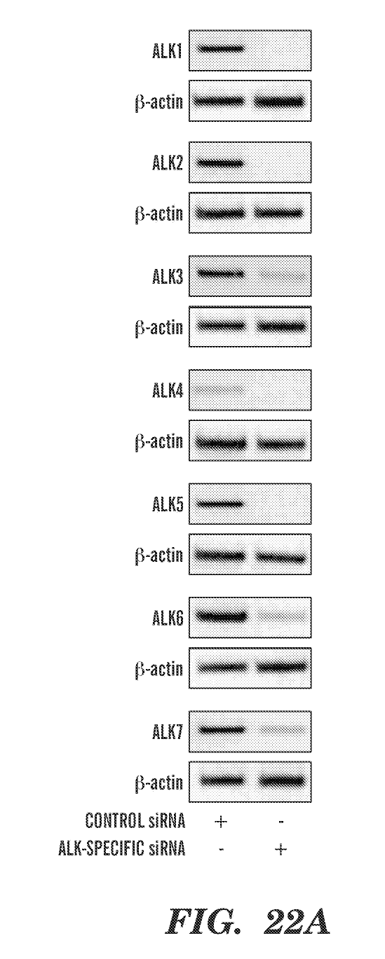

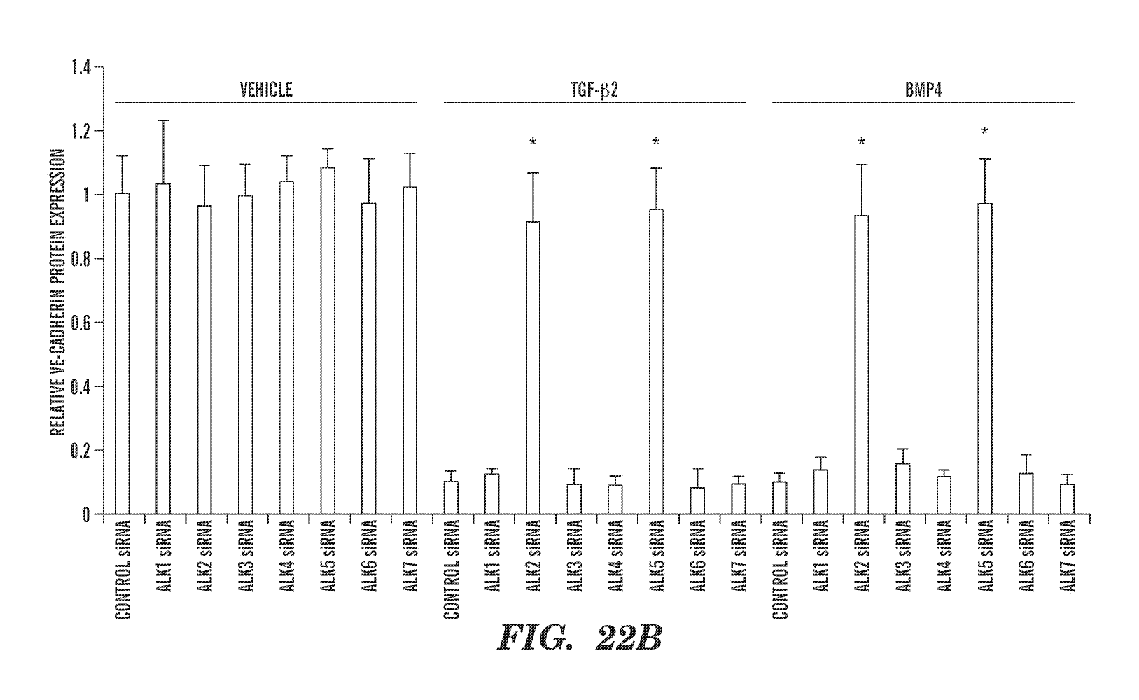

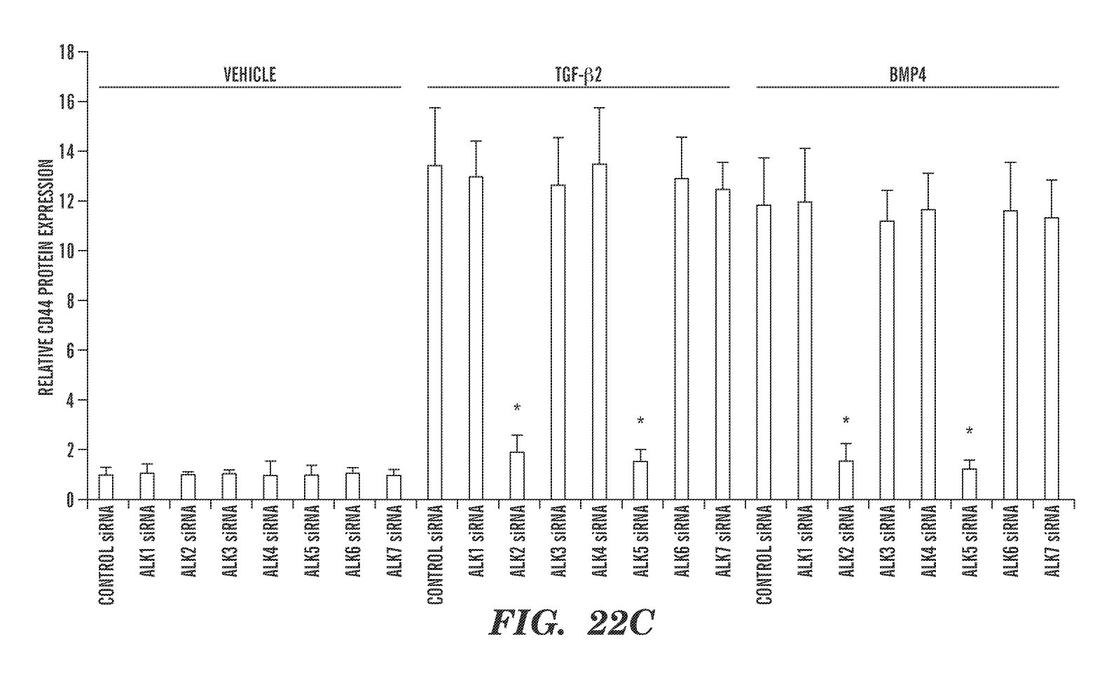

To further define the role of ALK receptors in EndMT, expression knockdown studies were performed using ALK-specific siRNA for all ALK receptors. EndMT-associated decrease in VE-cadherin and increase in CD44 expression induced by TGF-β2 or BMP4 were inhibited by ALK2 siRNA or ALK5 siRNA. Inhibition of ALK1, ALK3, ALK4, ALK6, or ALK7 expression had no effect on EndMT (FIG. 22). These data suggest that both ALK2 with ALK5 are necessary for EndMT.

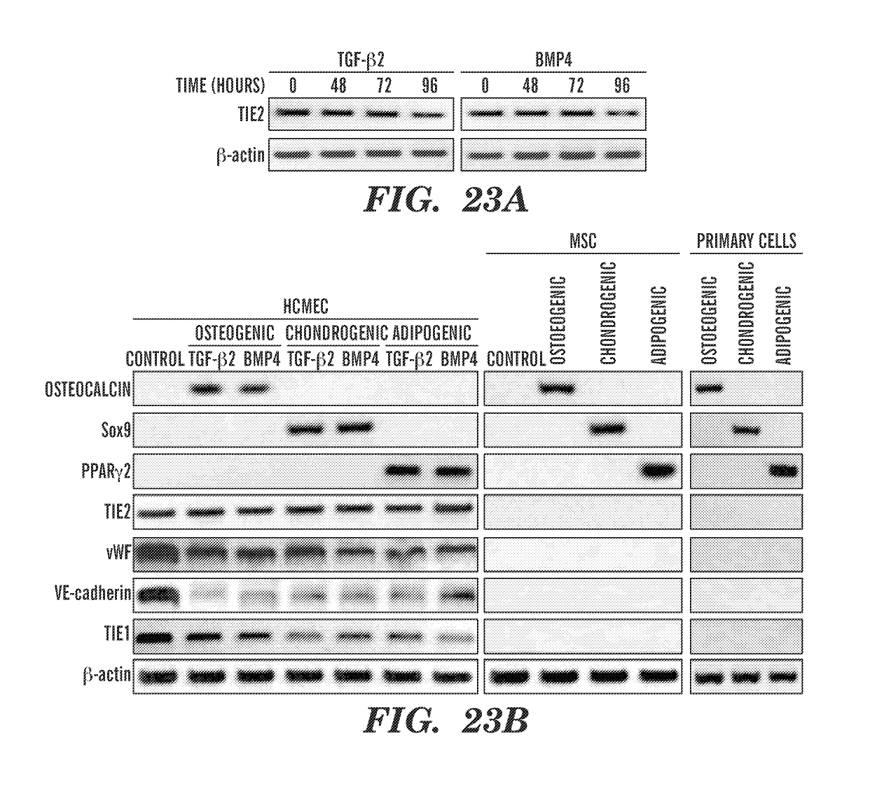

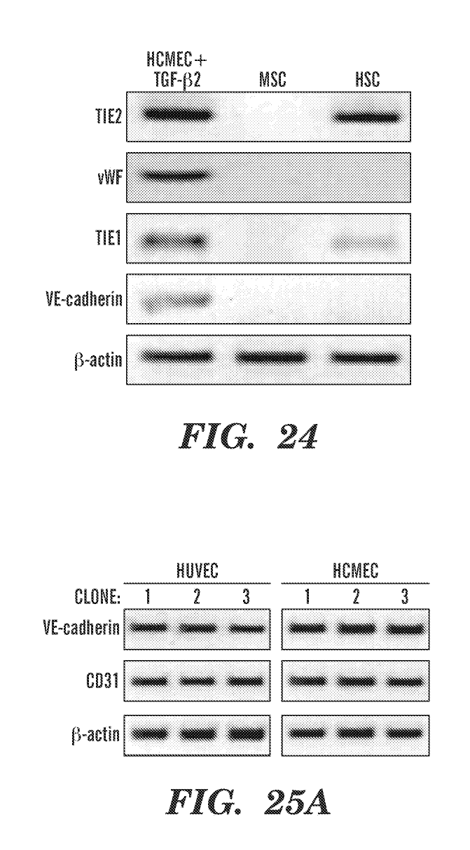

These experiments show that expression of endothelial markers vWF, TIE1, and VE-cadherin is reduced yet still detectable throughout EndMT. TIE2 levels remain constant through 48 h of treatment with TGF-β2 or BMP4, but a small decrease can be observed after 96 h of treatment (FIG. 23). Therefore, without being bound by theory, it is thought that all these markers can be used to detect endothelial-derived cells that differentiate into other cell types via the endothelial to stem-like cell mechanism. To confirm that osteoblasts, chondrocytes and adipocytes derived from endothelial cells express these markers, immunoblot analysis was performed on lysates collected from HCMEC cultures treated with vehicle, TGF-β2 or BMP4 for 48 h and grown in osteogenic medium for 7 d, chondrogenic medium for 14 days, or adipogenic medium for 7 d. Osteogenic medium induced expression of the osteoblast marker osteocalcin, chondrogenic medium increased expression of the chondrocyte marker SOX9, and adipogenic medium induced expression of the adipocyte marker PPARγ2. Bone marrow-derived mesenchymal stem cells (MSCs) grown in the same differentiation media showed positive expression of these proteins as well, but cells grown in conventional growth medium did not. Most importantly, MSCs and osteoblasts, chondrocytes, and adipocytes derived from MSCs showed no detectable expression of the endothelial markers TIE2, vWF, VE-cadherin, and TIE1. Primary human osteoblasts, chondrocytes, and adipocytes also showed no expression of these markers. However, osteoblasts, chondrocytes, and adipocytes derived from endothelial cells showed expression of these endothelial markers (FIG. 23B). In addition, while hematopoietic stem cells express TIE2 and low levels of TIE1, they do not express the endothelial-specific markers vWF or VE-cadherin (FIG. 24).

### Discussion

The findings discussed herein provide novel insights into the mechanism and potential roles of EndMT. First, the data demonstrate that EndMT results in generation of mesenchymal stem-like cells that can differentiate into multiple cell lineages. The current view is that EndMT produces fibroblastic cells that participate in specific developmental processes, in cancer progression and organ fibrosis (1), but the evidence for an endothelial derived stem-like cell broadens the scope for the role of EndMT in normal development, tissue repair, and disease. For example, the observation that chondrocytes and osteoblasts at sites of bone fracture repair stained positive with antibodies specific for endothelial markers (28), suggests that EndMT may contribute to the physiological process of fracture repair. Studies of the sources of chondrocytes and osteoblasts in fracture repair and the related process of distraction osteogenesis (29) should be of interest.

Second, the data indicate that activation/phosphorylation of ALK2 is necessary and sufficient for EndMT to occur in cells such as HUVECs and HCMECs under the in vitro conditions used in this study. The finding that a major fraction of the chondrocytes and osteoblasts in ectopic ossifying lesions of mice and humans with an activating mutation in ALK2 are likely derived from endothelial cells indicates that the process can occur also in vivo. The data demonstrate that TGF-β2 and BMP4 are stimulators of EndMT and confirm that BMP7 and vascular endothelial growth factor (VEGF) are inhibitors of EndMT (13,30,31) (FIG. 20B). The negative effect of VEGF on ALK2-mediated EndMT may explain why endochondral and not direct (membranous) bone formation occurs in the lesions of FOP patients. Chondrogenesis occurs in an anti-angiogenic, hypoxic environment (32) and this may be the condition that favors ALK2 mediated EndMT and chondrogenesis at lesion sites since hypoxia occurs as a result of inflammation (33). In contrast, bone formation requires angiogenesis and osteoblasts produce VEGF34. Factors that cause endothelial-derived cells to convert to chondrocytes in FOP are currently unknown. However, it is likely that inflammatory cytokines play a critical role since the ectopic ossifying lesions are triggered by inflammation (20).

Third, the data indicate that FOP, in which the hallmark is pathological bone formation, is a vascular disease based on conversion of endothelial cells into mesenchymal stem-like cells. Accumulation of mesenchymal cells is an early step in the formation of FOP lesions, and this was previously thought to be a result of fibroblast proliferation (23). The data raise the possibility that mesenchymal condensation prior to chondrogenesis is the result of EndMT to stem-like cells that condense and differentiate into chondrocytes by a process that mimics early steps in skeletal development (32). The data show that the majority of the chondrocytes and osteoblasts found in human FOP lesions, as well as in the lesions of mouse models of FOP, express endothelial markers. The origin of the relatively small fraction of cells that do not express these markers is unknown, but it is possible that these cells arise from mesenchymal stem cells recruited from the bone marrow or surrounding tissues.

### Methods

Cell Culture.

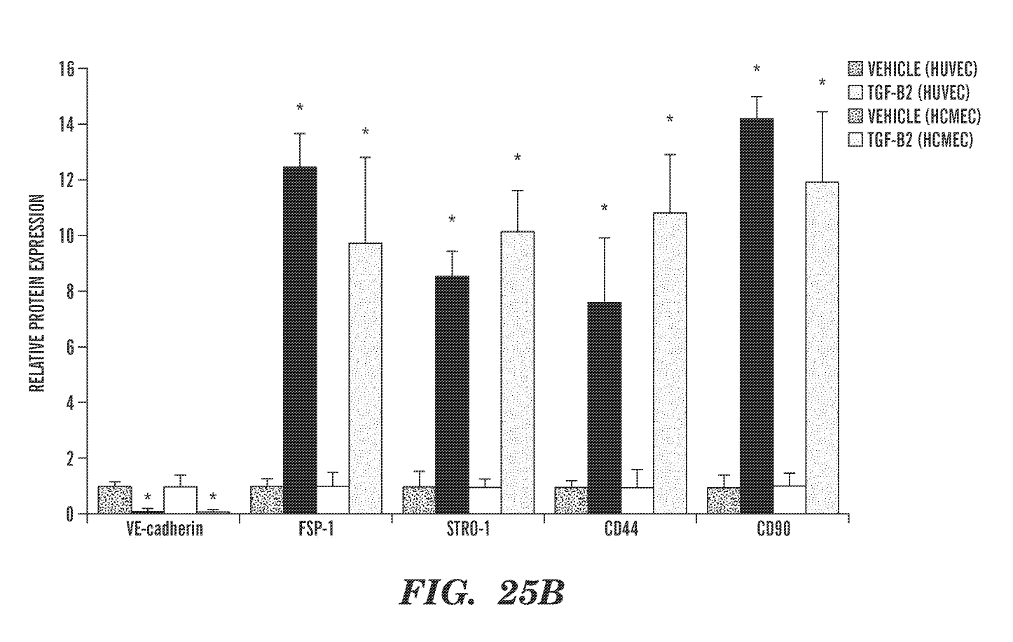

Human umbilical vein endothelial cells (HUVEC) and human cutaneous microvascular endothelial cells (HCMEC) isolated as previously described (35). Cells were tested for purity and found to express no markers for lymphatic endothelial cells or stromal cells (pericytes, smooth muscle cells, etc.) (36). This was reconfirmed by flow cytometry analysis (data not shown). Clonal populations were isolated with PYREX 8×8 mm cloning cylinders (Corning) and shown to respond in a manner identical to that of the cultures used for our experiments (FIG. 25B-D). Cells were grown in culture using EGM-2 medium (Cambrex), containing 10% FBS and 1% Penicillin/Streptomycin, followed by human endothelial serum free medium (Gibco) 24 h prior to all experimental conditions. Bone marrow derived mesenchymal stem cells (ScienCell Research Laboratories) were grown in mesenchymal stem cell medium (ScienCell Research Laboratories). Bone marrow derived hematopoietic stem cells (Lonza) were grown in HPGM medium (Lonza). Human corneal fibroblasts, from a stock provided by Dr. Elizabeth Hay (Harvard Medical School), were grown in RPMI medium, containing 10% FBS and 1% Penicillin/Streptomycin. Primary human osteoblasts, chondrocytes, and adipocytes and their respective growth media were obtained from Cell Applications, Inc. Recombinant TGF-β2, BMP4, and BMP7 proteins (R&D Systems) were added to the serum free culture medium for all relevant experiments at a concentration of 10 ng ml⁻¹. Recombinant VEGF (R&D Systems) was added to cultures at a concentration of 25 ng ml⁻¹. Dorsomorphin (Sigma-Aldrich) was added to cultures at a concentration of 5 μM. All experiments for this study were performed at minimum in triplicate.

Plasmids and Adenoviral Constructs.

Human ALK2 expression constructs were generated by insertion of full-length human ALK2 cDNA (GenBank NW_{—}001105) into the pcDNA 3.1D V5-His-TOPO vector (Invitrogen). The ALK2 R206H mutant construct was generated by site-directed mutagenesis of the normal ALK2 sequence using the Gene Tailor Site-Directed Mutagenesis System (Invitrogen). The oligonucleotides used to generate the mutant construct were: forward 5′-GTACAAAGAACAGTGGCTCACCAGATTACACTG-3′ (SEQ ID NO: 53); reverse 5′-GTGAGCCACTGTTCTTTGTACCAGAAAAGGAAG-3′ (SEQ ID NO: 54). SpeI and SphI sites were used to generate the adenoviral vectors through the Clontech Adeno-X System (University of Pennsylvania Vector Core). Expression from these constructs was confirmed by sequence analysis and immunoblot analysis. Viral constructs were added to cultures at a concentration of 20 pfu ml⁻¹.

Mice.

All procedures were reviewed and approved by the Institutional Animal Care and Use Committee at the University of Pennsylvania. Floxed caALK2 transgenic mice (37) were a gift from Dr. Yuji Mishina (University of Michigan). To induce expression of caALK2, AV-Cre (University of Pennsylvania Vector Core; 1×10¹¹ particles per mouse) was injected into the left hindlimbs of mice at one month of age. The contralateral limb was injected with empty vector as a control. Heterotopic bone and cartilage were detected by X-ray (Senographe DS technology, General Electric Medical Systems) at 14-21 d following AV-Cre injection. Tie2-Cre and IRG reporter mice were obtained from the Jackson Laboratory. Heterotopic ossification was induced in the offspring of crosses between the Tie2-Cre and IRG reporter mice with BMP4 (provided by Genetics Institute; currently Pfizer) injected at a concentration of 0.05 μg/μl, intramuscularly in growth factor-reduced Matrigel (BD Biosciences). Tissues were recovered at 7 and 14 d after implantation. Tissues were fixed in isopentane (2-methylbutane) as previously described (23) and cryosections were cut at 10 μm. Counterstaining of sections from Tie2-Cre; IRG reporter mice was performed using blue fluorescent AlexaFluor secondary antibodies (Invitrogen).

Positive control images of Tie2-Cre:EGFP localization was performed by immunohistochemistry of sections of muscle tissue from Tie2-Cre; IRG mice showing EGFP expression localized in blood vessels and confirmed by staining with antibodies specific for the endothelial marker VE-cadherin. No leakage or aberrant expression of the Tie2-Cre reporter was observed in any tissues (data not shown).

Human Tissues.

All FOP patient samples were obtained with informed consent and protocols approved by the Investigational Review Board of the University of Pennsylvania. All biopsies were obtained prior to a diagnosis of FOP since tissue trauma in FOP frequently induces episodes of heterotopic ossification. Normal human bone and cartilage tissue from the hip joint was provided by the Department of Pathology at the Beth Israel Deaconess Medical Center and samples were obtained with informed consent and protocols approved by the Investigational Review Board.

Immunoblotting, Immunoprecipitation, and Immunofluorescence.

Immunoassays were performed using the following antibodies at concentrations (and using protocols) recommended by the respective manufacturers: FSP-1 (H00006275-M01; Stressgen), phospho-Smad2 (3101), Smad2 (3122), phospho-Smad5 (9516), Smad5 (9517; Cell Signaling Technology), ALK1 (sc-19547), ALK2 (sc-25449), ALK3 (sc-20736), ALK4 (sc-31297), ALK5 (sc-398), ALK6 (sc-25455), ALK7 (sc-135001), phospho-tyrosine (sc-7020), VE-cadherin (sc-6458), TIE1 (sc-342), TIE2 (sc-324, sc-9026), STRO-1 (sc-47733), CD10 (58939), CD44 (sc-71220), CD71 (sc-32272), CD90 (sc-9163), CD117 (sc-17806), osteocalcin (sc-74495, sc-23790), SOX9 (sc-20095), PPARγ2 (sc-22022; Santa Cruz Biotechnology), osterix (ab22552), adiponectin (ab22554), N-cadherin (ab76057), NG2 (ab83508), vWF (ab68545; Abcam); CD31 (IR610; Dako), His (A00174; GenScript); α-SMA (A5228), β-actin (A1978; Sigma-Aldrich). Samples were run with Criterion precast SDS-PAGE Gels (Bio-Rad). HRP-conjugated IgG TrueBlot reagents (18-8814, 18-8816, 18-8817; eBioscience) were used at a dilution of 1:1000. TrueBlot IgG beads (eBioscience) were used for immunoprecipitation experiments. AlexaFluor secondary antibodies (Invitrogen) were used at a dilution of 1:200. Images were acquired using a Nikon 80i fluorescence microscope.

Flow Cytometry.

Endothelial cells were stained in suspension using antibodies specific for TIE2, FSP-1, STRO-1, α-SMA, NG2, and His (described above) and the protocols provided by their respective manufacturer. Flow cytometry was performed at the Harvard Medical School, Department of Pathology flow cytometry core facility using a FACSDCalibur (BD Biosciences) cell sorter isolating 30,000 cells per sample.

Multiplex ELISA.

LEF-1 (Cell Signaling Technology), VE-cadherin (sc-6458), CD44 (sc-71220), CD90 (sc-9163), Snail (sc-10433), ZEB-1 (sc-134159; Santa Cruz Biotechnology), FSP-1 (H00006275-M01; Stressgen), SIP-1 (AV33694; Sigma-Aldrich), Slug (ab27568), and Twist (ab49254; Abcam) antibodies were conjugated to Bio-Plex carboxylated beads with unique optical codes using the Bio-Plex Amine Coupling Kit (BioRad). β-actin antibody (A1978; Sigma-Aldrich) was also conjugated to Bio-Plex carboxylated beads to be used as an internal control. Samples were run on a Luminex 200 multiplex testing system (Luminex) using the Universal Cell Signaling Assay Kit and protocol (Millipore). Experimental values were divided by the β-actin control values to provide normalized data.

Cell Differentiation.

Cells were grown in StemPro osteogenic, chondrogenic, or adipogenic culture medium (Invitrogen). Alkaline phosphatase staining was performed using the alkaline phosphatase kit and protocol (Sigma-Aldrich) on cultures grown in osteogenic medium for 7 d to detect osteoblasts. Alizarin Red (Sigma-Aldrich) staining was performed for 30 min on cultures grown in osteogenic medium for 21 d to detect matrix calcification. Alcian Blue (Sigma) staining was used to stain chondrocyte proteoglycans for 5 minutes in cultures grown in chondrogenic medium for 14 d. Oil Red O (Sigma-Aldrich) staining was performed for 15 min on cultures grown in adipogenic medium for 7 d. For in vivo analysis, endothelial cells were labeled with fluorescent quantum dots using the Qtracker 525 Cell Labeling Kit (Invitrogen). Cells were treated in culture to induce EndMT then absorbed into OPLA polylactic acid scaffolds (BD Biosciences). Scaffolds were surgically implanted subcutaneously into immunodeficient nude mice (Nu/Nu strain; Charles River Laboratories). Local injection of StemPro differentiation medium (described above) was performed in the area of the implants every 72 h for 6 weeks. Scaffolds were cryosectioned and stained with Alizarin Red, Alcian Blue, or Oil Red O as described above. All procedures were reviewed and approved by the Institutional Animal Care and Use Committee at Harvard Medical School.

RNA Interference.

siRNA gene expression knockdown studies were performed using the TriFECTa RNAi kit (Integrated DNA Technologies) and corresponding protocol. Each 27 mer siRNA duplex was transfected into cells using X-tremeGene siRNA transfection reagent (Roche) following the manufacturer's guidelines. siRNA was synthesized (Integrated DNA Technologies) with the following sequences:

AKL1:
(SEQ ID NO: 55)
5′-CUGGGCUAUUGAAUCACUUUAGGCUUC-3′;
ALK2:
(SEQ ID NO: 56)
5'-GCAACACUGUCCAUUCUUCUUAACCAG-3′;
ALK3:
(SEQ ID NO: 57)
5'-CAUCUCAUGAAUUCCAAGACAGUAUUA-3′;
ALK4:
(SEQ ID NO: 58)
5'-AUGAGGGAUCUUCCAUGUCCAGUCUCU-3′;
ALK5:
(SEQ ID NO: 59)
5-CUCAGAAUGUUCUUUAGCUACCACCUC-3′;
ALK6:
(SEQ ID NO: 60)
5'-AUCUGAAUCUGCUUAGCUAUAGUCCUU-3′;
ALK7:
(SEQ ID NO: 61)
5'-ACUUAAAUACUGUACUGUCUUAUCUUU-3′;
negative control:
(SEQ ID NO: 62)
5'-UCACAAGGGAGAGAAAGAGAGGAAGGA-3′.

Statistical Analyses.

One-way analysis of variance (ANOVA) was performed and confirmed with two-tailed paired student's t test using GraphPad Prism 4 software. P values less than 0.05 were considered significant.

### REFERENCES EXAMPLE 2

1. Potenta, S., Zeisberg, E. & Kalluri, R. The role of endothelial-to-mesenchymal transition in cancer progression. Br. J. Cancer 99, 1375-1379 (2008).
2. Akhurst, R. J. & Derynck, R. TGF-beta signaling in cancer—a double-edged sword. Trends Cell Biol. 11, S44-S51 (2001).
3. Thiery, J. P. Epithelial-mesenchymal transitions in tumor progression. Nat. Rev. Cancer 2, 442-454 (2002).
4. Thiery, J. P. Epithelial-mesenchymal transitions in development and pathologies. Curr. Opin. Cell Biol. 15, 740-746 (2003).
5. Hay, E. D. The mesenchymal cell, its role in the embryo, and the remarkable signaling mechanisms that create it. Dev. Dyn. 233, 706-720 (2005).
6. Boyer, A. S. et al. TGFbeta2 and TGFbeta3 have separate and sequential activities during epithelial-mesenchymal cell transformation in the embryonic heart. Dev. Biol. 208, 530-545 (1999).
7. Lai, Y. T. et al. Activin receptor-like kinase 2 can mediate atrioventricular cushion transformation. Dev. Biol. 222, 1-11 (2000).
8. Camenisch, T. D. et al. Temporal and distinct TGFbeta ligand requirements during mouse and avian endocardial cushion morphogenesis. Dev. Biol. 248, 170-181 (2002).
9. Wang, J. et al. Atrioventricular cushion transformation is mediated by ALK2 in the developing mouse heart. Dev. Biol. 286, 299-310 (2005).
10. Okagawa, H., Markwald, Y. & Sugi, Y. Functional BMP receptor in endocardial cells is required in atrioventricular cushion mesenchymal cell formation in the chick. Dev. Biol. 306, 179-192 (2007).
11. Azhar, M. et al. Ligand-specific function of transforming growth factor beta in epithelial-mesenchymal transition in heart development. Dev Dyn. 238, 431-442 (2009).
12. Zeisberg, E. M., Potenta, S., Xie, L., Zeisberg, M. & Kalluri, R. Discovery of endothelial to mesenchymal transition as a source for carcinoma-associated fibroblasts. Cancer Res. 67, 10123-10128 (2007).
13. Zeisberg, E. M. et al. Endothelial-to-mesenchymal transition contributes to cardiac fibrosis. Nat. Med. 13, 952-961 (2007).
14. Zeisberg, E. M., Potenta, S. E., Sugimoto, H., Zeisberg, M. & Kalluri, R. Fibroblasts in kidney fibrosis emerge via endothelial-to-mesenchymal transition. J. Am. Soc. Nephrol. 19, 2282-2287 (2008).
15. Kizu, A., Medici, D. & Kalluri, R. Endothelial-mesenchymal transition as a novel mechanism for generating myofibroblasts during diabetic nephropathy. Am. J. Pathol. 175, 1371-1373 (2009).
16. Li, J., Qu, X. & Bertram, J. F. Endothelial-myofibroblast transition contributes to the early development of diabetic renal interstitial fibrosis in streptozotocin-induced diabetic mice. Am. J. Pathol. 175, 1380-1388 (2009).
17. Mironov, V. et al. Endothelial-mesenchymal transformation in atherosclerosis: a recapitulation of embryonic heart tissue morphogenesis. Ann. Biomed. Eng. 23, S29A (1995).
18. Arciniegas, E., Frid, M. G., Douglas, I. S. & Stenmark, K. R. Perspectives on endothelial-to-mesenchymal transition: potential contribution to vascular remodeling in chronic pulmonary hypertension. Am. J. Physiol. Lung Cell Mol. Physiol. 293, L1-8 (2007).
19. Lee, J. G. & Kay, E. P. FGF-2-mediated signal transduction during endothelial mesenchymal transformation in corneal endothelial cells. Exp. Eye Res. 83, 1309-1316 (2006).
20. Shore, E. M. & Kaplan, F. S. Insights from a rare genetic disorder of extra-skeletal bone formation, fibrodysplasia ossificans progressiva (FOP). Bone 43, 427-433 (2008).
21. Kaplan, F. S. et al. Skeletal metamorphosis in fibrodysplasia ossificans progressiva (FOP). J. Bone Miner. Metab. 26, 521-530 (2008).
22. Shore, E. M. et al. A recurrent mutation in the BMP type I receptor ACVR1 causes inherited and sporadic fibrodysplasia ossificans progressiva. Nat. Genet. 38, 525-527 (2006).
23. Lounev, V. Y. et al. Identification of progenitor cells that contribute to heterotopic skeletogenesis. J. Bone Joint Surg. Am. 91, 652-663 (2009).
24. Kalluri, R. & Weinberg, R. A. The basics of epithelial-mesenchymal transition. J. Clin. Invest. 119, 1420-1428 (2009).
25. Chamberlain, G., Fox, J., Ashton, B. & Middleton, J. Concise review: mesenchymal stem cells: their phenotype, differentiation capacity, immunological features, and potential for homing. Stem Cells 25, 2739-2749 (2007).
26. Chen, D., Zhao, M. & Mundy, G. R. Bone morphogenetic proteins. Growth Factors 22, 233-241 (2004).
27. Mercado-Pimentel, M. E. & Runyan R. B. Multiple transforming growth factor-beta isoforms and receptors function during epithelial-mesenchymal cell transformation in the embryonic heart. Cells Tissues Organs 185, 146-156 (2007).
28. Lewinson, D. et al. Expression of vascular antigens by bone cells during bone regeneration in a membranous bone distraction system. Histochem. Cell Biol. 116, 381-388 (2001).
29. Ilizarov, G. A. The principles of the Ilizarov method. Bull. Hosp. Jt. Dis. Orthop. Inst. 48, 1-11 (1988).
30. Paruchuri, S. et al. Human pulmonary valve progenitor cells exhibit endothelial/mesenchymal plasticity in response to vascular endothelial growth factor-A and transforming growth factor-beta2. Circ. Res. 99, 861-869 (2006).
31. Goumans, M. J., van Zonneveld, A. J. & ten Dijke, P. Transforming growth factor beta-induced endothelial-to-mesenchymal transition: a switch to cardiac fibrosis? Trends Cardiovasc. Med. 18, 293-298 (2008).
32. Amarilio, R. et al. HIF1alpha regulation of Sox9 is necessary to maintain differentiation of hypoxic prechondrogenic cells during early skeletogenesis. Development 134, 3917-3928 (2007).
33. Karhausen, J., Haase, V. H. & Colgan, S. P. Inflammatory hypoxia: role of hypoxia-inducible factor. Cell Cycle 4, 256-258 (2005).
34. Dai, J. & Rabie, A. B. VEGF: an essential mediator of both angiogenesis and endochondral ossification. J. Dent. Res. 86, 937-950 (2007).
35. Boye, E. et al. Clonality and altered behavior of endothelial cells from hemangiomas. J. Clin. Invest. 107, 745-752 (2001).
36. Jinnin, M. et al. Suppressed NFAT-dependent VEGFR1 expression and constitutive VEGFR2 signaling in infantile hemangioma. Nat. Med. 14, 1236-1246 (2008).
37. Fukuda, T. et al. Generation of a mouse with conditionally activated signaling through the BMP receptor, ALK2. Genesis 44, 159-167 (2006).

### Example 3

Hemangiomas are the most common tumors of infancy. These vascular neoplasms occur in approximately 10% of Caucasian population. Infantile hemangiomas appear approximately 2 weeks after birth then grow rapidly over the first year of life by clonal expansion of vascular endothelial cells (Boye et al., 2001). Tumor growth then ceases, followed by slow involution (regression) over the next 7-10 years that causes disappearance of the lesion (Boye and Olsen, 2009).

The cause of hemangioma involution is currently unknown. Some apoptosis has been observed in involuting hemangiomas compared to proliferating hemangiomas (Razon et al., 1998). However, adipogenesis appears to be the predominant mechanism of hemangioma regression, as the vascular endothelial tumor is replaced by a mass of fat tissue. A comparison of proliferating and involuting hemangiomas indicates that proliferating hemangioma is composed of vascular endothelial tissue while the involuting hemangioma is composed primarily of adipose tissue. Hematoxylin and Eosin staining (H & E staining) of proliferating hemangioma tissue indicates no adipocytes present, while H & E staining of involuting hemangioma tissue indicates a predominant composition of adipocytes (Yu et al., (2006)). Previous studies suggested that mesenchymal stem cells recruited into proliferating hemangioma tissues from the bone marrow might be a source of the adipocytes seen in hemangioma involution. Although mesenchymal stem cells isolated from hemangiomas can be differentiated into adipocytes in vitro in the presence of adipogenic stimuli, mesenchymal stem cells make up less than 1% of the total population of proliferating hemangiomas (Yu et al., 2006) so it is difficult to understand how a relatively small number of cells could result in adipogenic transformation of nearly the entire tumor. Therefore, it was hypothesize that endothelial cells are the primary source of adipocytes in involuting hemangiomas.

To determine whether adipocytes formed during hemangioma involution are derived from endothelial cells, immunohistochemistry was performed on tissue sections of involuting hemangiomas and also on normal human subcutaneous adipose tissue. Tissue sections of involuting hemangiomas or normal human subcutaneous adipose tissue were stained using antibodies against the endothelial markers TIE2, vWF, or VE-cadherin, with co-staining for adiponectin. In involuting hemangiomas, significant amounts of adipose tissue stained positive for TIE2, vWF, and VE-cadherin along with the adipocyte marker adiponectin. There was no indication of positive staining for the normal human subcutaneous adipose tissue for any of the endothelial markers, but normal staining for adiponectin. These results indicate that adipose tissue in involuting hemangiomas is endothelial derived.

To determine whether endothelial-mesenchymal transition (EndMT) occurs during hemangioma involution, immunohistochemistry was performed on patient tissue sections of proliferating and involuting hemangiomas. Tissue sections of proliferating and involuting menangiomas were stained using antibodies specific for the endothelial markers TIE2, vWF, or VE-cadherin and co-stained with antibodies specific for the mesenchymal marker FSP-1. Co-staining, indicative of co-expression, for the endothelial-markers TIE2, vWF, or VE-cadherin with the mesenchymal marker FSP-1 was observed in involuting hemangiomas, but was not observed in proliferating hemangiomas. The absence of co-expression of these markers in proliferating hemangiomas indicates no evidence of EndMT, whereas the high amounts of co-expression observed in involuting hemangiomas indicates EndMT occurs during hemangioma involution. The occurrence of this phenomena in nature is further support for the ability to convert vascular endothelial cells into adipocytes by the methods described herein.

### REFERENCES EXAMPLE 3

1. Boye, E., Yu, Y., Paranya, G., Mulliken, J. B., Olsen, B. R., and Bischoff, J. (2001). Clonality and altered behavior of endothelial cells from hemangiomas. J. Clin. Invest. 107: 745-752.
2. Boye, E., and Olsen, B. R. (2009). Signaling mechanisms in infantile hemangioma. Curr. Opin. Hematol. 16: 202-208.
3. Razon, M. J., Kräling, B. M., Mulliken, J. B., and Bischoff, J. (1998). Increased apoptosis coincides with onset of involution in infantile hemangioma. Microcirculation 5: 189-195.
4. Yu, Y., Fuhr, J., Boye, E., Gyorffy, S., Soker, S., Atala, A., Mulliken, J. B., and Bischoff, J. (2006). Mesenchymal stem cells and adipogenesis in hemangioma involution. Stem Cells 24: 1605-1612.

### Example 4

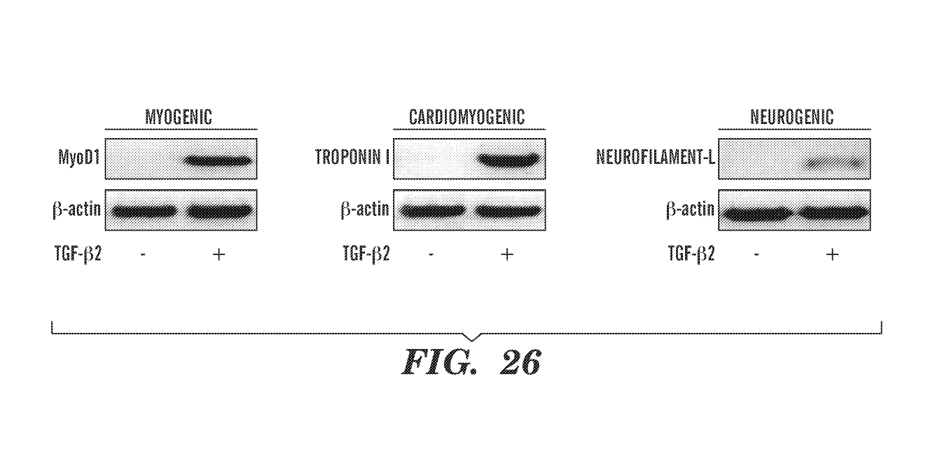

To further demonstrate that endothelial-derived mesenchymal stem-like cells can be differentiated into skeletal muscle cells (myocytes), heart muscle cells (cardiomyocytes), or nerve cells (neurons), human cutaneous microvascular endothelial cells (HCMEC) were treated with vehicle or TGF-β2 for 48 hours, as described above for generation of mesenchymal stem-like cells, followed by exposure to either myogenic, cardiomyogenic, or neurogenic culture medium for 14 days. After appropriate incubation periods in the respective differentiation medium, cells were lysed with detergent buffer and immunoblotting was performed using antibodies specific for the myocyte marker MyoD1, cardiomyocyte marker cardiac troponin I, or neuronal marker neurofilament-L. Vehicle treated cells (control) cultured under myogenic, cardiomyogenic, or neurogenic culture conditions, showed no expression of these markers, but endothelial cells converted to mesenchymal stem-like cells with TGF-β2 and grown under the myogenic, cardiomyogenic, or neurogenic culture conditions did express these markers (FIG. 26). These data further show successful differentiation of endothelial-derived mesenchymal stem-like cells into myocytes, cardiomyocytes, and neurons using the methods described herein.

Cardiomyogenic medium was purchased from Millipore Inc., (SCM-102).

Myogenic medium consisted of human endothelial serum-free medium supplemented with 5% horse serum, 0.1 μM dexamethasone, and 50 μM hydrocortisone (previously described by Gang et al, 2004).

Neurogenic medium consisted human endothelial serum-free medium supplemented with 100 ng/ml bFGF (previously described by Jiang et al., 2002). Endothelial cells converted into mesenchymal stem-like cells by treatment with 10 ng/ml TGF-β2 for 48 hours were plated on fibronectin-coated polystyrene wells in the neurogenic medium.

### REFERENCES EXAMPLE 4

Gang, E. J., Jeong, J. A., Hong, S. H., Hwang, S. H., Kim, S. W., Yang, I. H., Ahn, Chiyoung, A., Han, H., and Kim, H. (2004). Skeletal myogenic differentiation of mesenchymal stem cells isolated from human umbilical cord blood. Stem Cells 22, 617-624.
Jiang, Y., Jahagirdar, B. N., Reinhardt, R. L., Schwartz, R. E., Keene, C. D., Ortiz-Gonzales, X. R., Reyes, M., Lenvik, T., Lund, T., Blackstad, M., Du, J., Aldrich, S., Lisberg, A., Low, W. C., Largaespada, D. A., and Verfaillie, C. M. (2002). Pluripotency of mesenchymal stem cells derived from adult marrow. Nature 418, 41-49.

1. A method of producing multipotent cells from endothelial cells, comprising, activating ALK2 in the endothelial cells, in a serum starved environment, to thereby produce multipotent cells.

2. The method of claim 1, further comprising subjecting the endothelial cells to a threshold period of serum starvation prior to activating ALK2.

3. The method of claim 1, wherein activating ALK2 is by contacting the endothelial cells with an agent selected from the group consisting of TGFβ-2 or an analog, derivative or functional fragment thereof, BMP4 or an analog, derivative or functional fragment thereof, and a combination thereof.

4. The method of claim 3, wherein the agent is contacted to the endothelial cells at a concentration of about 10 ng/ml.

5. The method of claim 1, wherein activating ALK2 is for at least about 48 hours.

6. The method of claim 2, wherein the threshold period of serum starvation is for at least about 24 hours.

7. The method of claim 1, wherein the endothelial cells are selected from the group consisting of primate, equine, bovine, porcine, canine, feline, and rodent.

8. The method of claim 1, wherein the endothelial cells are human.

9. The method of claim 1, wherein the endothelial cells are primary vascular or primary microvascular endothelial cells.

10. The method of claim 1, wherein the endothelial cells are isolated.

11. The method of claim 1, wherein the endothelial cells are primary human umbilical vein endothelial cells (HUVEC) or primary human cutaneous microvascular endothelial cells (HCMEC).

12. The method of claim 1, wherein activation of ALK2 significantly decreases expression of VE-cadherein of the cells, significantly increases expression of one or more of STRO-1, FSP-1, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, CD124, or a combination thereof.

13.-18. (canceled)

19. An isolated multipotent cell, or population thereof, wherein the multipotent cell expresses transcripts for STRO-1, FSP-1, TIE-2, α-SMA, N-cadherin, fibronectin (FN1), Snail (SNAI1), Slug (SNAI2), ZEB-1, SIP-1, LEF-1, Twist, CD10, CD13, CD44, CD73, CD90, CD120A, or CD124, or combinations thereof, and has a normal karyotype.

20. (canceled)

21. The isolated multipotent cell of claim 19, that is produced by the method of claim 1.

22. (canceled)

23. The isolated multipotent cell or population thereof, of claim 19 that has fibroblast-like morphology.

24. The isolated multipotent cell or population thereof, of claim 21 that is human.

25. An isolated cell or population thereof, that expresses one or more cell-type specific markers in combination with TIE-2, wherein the cell-type specific markers are selected from the group consisting of osteoblast specific markers, chondrocyte specific markers, adipocyte specific markers, neuronal specific markers, myocyte specific markers, and cardiomyocyte specific markers.

26.-32. (canceled)

33. The isolated cell or population thereof, of claim 25 that is produced by the method of claim 45.

34.-44. (canceled)

45. The method of claim 1, further comprising differentiating the multipotent cells by incubating the multipotent cells in culture medium selected from the group consisting of osteogenic culture medium for a period sufficient to induce differentiation into osteoblast-like cells, chondrogenic culture medium for a period sufficient to induce differentiation into chondrocyte-like cells, adipogenic culture medium for a period sufficient to induce differentiation into adipocyte-like cells, neuralgenic culture medium for a period sufficient to induce differentiation into neural-like cells, myogenic culture medium for a period sufficient to induce differentiation into myocyte-like cells, and cardiomyogenic culture medium for a period sufficient to induce differentiation into cardiomyocyte-like cells.


SEQUENCE LISTING (62 sequences)

SEQ ID NO: 1 - DNA, 33 bp, Artificial Sequence
  Description of Artificial Sequence Synthetic oligonucleotide
gtacaaagaacagtggctcaccagattacactg

SEQ ID NO: 2 - DNA, 33 bp, Artificial Sequence
  Description of Artificial Sequence Synthetic oligonucleotide
gtgagccactgttctttgtaccagaaaaggaag

SEQ ID NO: 3 - DNA, 20 bp, Artificial Sequence
  Description of Artificial Sequence Synthetic primer
tctttcttggtttgatcctg

SEQ ID NO: 4 - DNA, 20 bp, Artificial Sequence
  Description of Artificial Sequence Synthetic primer
gcatcaagcacgtgtctgaa

SEQ ID NO: 5 - DNA, 21 bp, Artificial Sequence
  Description of Artificial Sequence Synthetic primer
gtccccatctatgagggctat

SEQ ID NO: 6 - DNA, 21 bp, Artificial Sequence
  Description of Artificial Sequence Synthetic primer
gcatttgcggtggacaatgga

SEQ ID NO: 7 - DNA, 27 bp, Artificial Sequence
  Description of Artificial Sequence Synthetic primer
cacccaacatgtttacaatcaacaatg

SEQ ID NO: 8 - DNA, 27 bp, Artificial Sequence
  Description of Artificial Sequence Synthetic primer
ctgcagcaacagtaaggacaaacatcc

SEQ ID NO: 9 - DNA, 22 bp, Artificial Sequence
  Description of Artificial Sequence Synthetic primer
cccaccgtctcaacatgcttag

SEQ ID NO: 10 - DNA, 24 bp, Artificial Sequence
  Description of Artificial Sequence Synthetic primer
ctcggcttcctccataacaagtac

SEQ ID NO: 11 - DNA, 24 bp, Artificial Sequence
  Description of Artificial Sequence Synthetic primer
gctgcaggactctaatccagagtt

SEQ ID NO: 12 - DNA, 23 bp, Artificial Sequence
  Description of Artificial Sequence Synthetic primer
gacagagtcccagatgagcattg

SEQ ID NO: 13 - DNA, 20 bp, Artificial Sequence
  Description of Artificial Sequence Synthetic primer
tgttgcagtgagggcaagaa

SEQ ID NO: 14 - DNA, 20 bp, Artificial Sequence
  Description of Artificial Sequence Synthetic primer
gaccctggttgcttcaagga

SEQ ID NO: 15 - DNA, 21 bp, Artificial Sequence
  Description of Artificial Sequence Synthetic primer
ttcaaacccatagtggttgct

SEQ ID NO: 16 - DNA, 21 bp, Artificial Sequence
  Description of Artificial Sequence Synthetic primer
tgggagataccaaaccaactg

SEQ ID NO: 17 - DNA, 18 bp, Artificial Sequence
  Description of Artificial Sequence Synthetic primer
caagaggcgcaaacaagc

SEQ ID NO: 18 - DNA, 20 bp, Artificial Sequence
  Description of Artificial Sequence Synthetic primer
ggttggcaataccgtcatcc

SEQ ID NO: 19 - DNA, 22 bp, Artificial Sequence
  Description of Artificial Sequence Synthetic primer
ccgaagaggaaggcgatttagc

SEQ ID NO: 20 - DNA, 20 bp, Artificial Sequence
  Description of Artificial Sequence Synthetic primer
ggtcccttgttgtagaggcc

SEQ ID NO: 21 - DNA, 20 bp, Artificial Sequence
  Description of Artificial Sequence Synthetic primer
ggagtccgcagtcttacgag

SEQ ID NO: 22 - DNA, 20 bp, Artificial Sequence
  Description of Artificial Sequence Synthetic primer
tctggaggacctggtagagg

SEQ ID NO: 23 - DNA, 21 bp, Artificial Sequence
  Description of Artificial Sequence Synthetic primer
aacatggatgccaccactgag

SEQ ID NO: 24 - DNA, 21 bp, Artificial Sequence
  Description of Artificial Sequence Synthetic primer
cacatatgctgtacaagcctc

SEQ ID NO: 25 - DNA, 21 bp, Artificial Sequence
  Description of Artificial Sequence Synthetic primer
aagctcaactacaccctcagc

SEQ ID NO: 26 - DNA, 21 bp, Artificial Sequence
  Description of Artificial Sequence Synthetic primer
gggtgtgtcataatgaccagc

SEQ ID NO: 27 - DNA, 20 bp, Artificial Sequence
  Description of Artificial Sequence Synthetic primer
cggacaccatggacaagttt

SEQ ID NO: 28 - DNA, 20 bp, Artificial Sequence
  Description of Artificial Sequence Synthetic primer
gaaagccttgcagaggtcag

SEQ ID NO: 29 - DNA, 20 bp, Artificial Sequence
  Description of Artificial Sequence Synthetic primer
aaggaaggggaagaacagga

SEQ ID NO: 30 - DNA, 20 bp, Artificial Sequence
  Description of Artificial Sequence Synthetic primer
ggcagagctgatggaatctc

SEQ ID NO: 31 - DNA, 19 bp, Artificial Sequence
  Description of Artificial Sequence Synthetic primer
caggtgatccgtccggcaa

SEQ ID NO: 32 - DNA, 16 bp, Artificial Sequence
  Description of Artificial Sequence Synthetic primer
gagaggcgtggagacc

SEQ ID NO: 33 - DNA, 21 bp, Artificial Sequence
  Description of Artificial Sequence Synthetic primer
cctacttgtacaatgactgtc

SEQ ID NO: 34 - DNA, 19 bp, Artificial Sequence
  Description of Artificial Sequence Synthetic primer
tgcatggcaggtgcacacg

SEQ ID NO: 35 - DNA, 22 bp, Artificial Sequence
  Description of Artificial Sequence Synthetic primer
aaatcgtgaactttgtctccgt

SEQ ID NO: 36 - DNA, 21 bp, Artificial Sequence
  Description of Artificial Sequence Synthetic primer
cccagtgccctctactctcat

SEQ ID NO: 37 - DNA, 21 bp, Artificial Sequence
  Description of Artificial Sequence Synthetic primer
aggcgctacctgtatcaatgg

SEQ ID NO: 38 - DNA, 19 bp, Artificial Sequence
  Description of Artificial Sequence Synthetic primer
tagaccgggccgtagaagc

SEQ ID NO: 39 - DNA, 22 bp, Artificial Sequence
  Description of Artificial Sequence Synthetic primer
aacccccagctgcccacctacc

SEQ ID NO: 40 - DNA, 22 bp, Artificial Sequence
  Description of Artificial Sequence Synthetic primer
gacgctccagctcatccgaacg

SEQ ID NO: 41 - DNA, 22 bp, Artificial Sequence
  Description of Artificial Sequence Synthetic primer
ttcagctatggagatgacaatc

SEQ ID NO: 42 - DNA, 20 bp, Artificial Sequence
  Description of Artificial Sequence Synthetic primer
agagtcctagagtgactgag

SEQ ID NO: 43 - DNA, 20 bp, Artificial Sequence
  Description of Artificial Sequence Synthetic primer
agacctttgggctgccttat

SEQ ID NO: 44 - DNA, 20 bp, Artificial Sequence
  Description of Artificial Sequence Synthetic primer
tagcctccctcactccaaga

SEQ ID NO: 45 - DNA, 21 bp, Artificial Sequence
  Description of Artificial Sequence Synthetic primer
catgaccaggaaaccacgact

SEQ ID NO: 46 - DNA, 17 bp, Artificial Sequence
  Description of Artificial Sequence Synthetic primer
tgaatgctgagcggtat

SEQ ID NO: 47 - DNA, 20 bp, Artificial Sequence
  Description of Artificial Sequence Synthetic primer
aggagcagagcaaagaggtg

SEQ ID NO: 48 - DNA, 20 bp, Artificial Sequence
  Description of Artificial Sequence Synthetic primer
aggactcagggtggttcagc

SEQ ID NO: 49 - DNA, 19 bp, Artificial Sequence
  Description of Artificial Sequence Synthetic primer
gaaggtgaaggtcggagtc

SEQ ID NO: 50 - DNA, 20 bp, Artificial Sequence
  Description of Artificial Sequence Synthetic primer
gaagatggtgatgggatttc

SEQ ID NO: 51 - RNA, 27 nt, Artificial Sequence
  Description of Artificial Sequence Synthetic oligonucleotide
gcaacacuguccauucuucuuaaccag

SEQ ID NO: 52 - RNA, 27 nt, Artificial Sequence
  Description of Artificial Sequence Synthetic oligonucleotide
ucacaagggagagaaagagaggaagga

SEQ ID NO: 53 - DNA, 33 bp, Artificial Sequence; identical to SEQ ID NO: 1
  Description of Artificial Sequence Synthetic oligonucleotide

SEQ ID NO: 54 - DNA, 33 bp, Artificial Sequence; identical to SEQ ID NO: 2
  Description of Artificial Sequence Synthetic oligonucleotide

SEQ ID NO: 55 - RNA, 27 nt, Artificial Sequence
  Description of Artificial Sequence Synthetic oligonucleotide
cugggcuauugaaucacuuuaggcuuc

SEQ ID NO: 56 - RNA, 27 nt, Artificial Sequence
  Description of Artificial Sequence Synthetic oligonucleotide
gcaacacuguccauucuucuuaaccag

SEQ ID NO: 57 - RNA, 27 nt, Artificial Sequence
  Description of Artificial Sequence Synthetic oligonucleotide
caucucaugaauuccaagacaguauua

SEQ ID NO: 58 - RNA, 27 nt, Artificial Sequence
  Description of Artificial Sequence Synthetic oligonucleotide
augagggaucuuccauguccagucucu

SEQ ID NO: 59 - RNA, 27 nt, Artificial Sequence
  Description of Artificial Sequence Synthetic oligonucleotide
cucagaauguucuuuagcuaccaccuc

SEQ ID NO: 60 - RNA, 27 nt, Artificial Sequence
  Description of Artificial Sequence Synthetic oligonucleotide
aucugaaucugcuuagcuauaguccuu

SEQ ID NO: 61 - RNA, 27 nt, Artificial Sequence
  Description of Artificial Sequence Synthetic oligonucleotide
acuuaaauacuguacugucuuaucuuu

SEQ ID NO: 62 - RNA, 27 nt, Artificial Sequence
  Description of Artificial Sequence Synthetic oligonucleotide
ucacaagggagagaaagagaggaagga